(19) United States
(12) Patent Application Publication
(10) Pub. No.: US 2017/0198044 A1
(43) Pub. Date: Jul. 13, 2017

(54) COMPOSITIONS AND METHODS FOR SAFE AND EFFECTIVE IMMUNOTHERAPY

(71) Applicant: TheraMAB LLC, Moscow (RU)

(72) Inventors: Alexey MATSKEVICH, Moscow (RU); Dmitry TYRSIN, Moscow (RU); Sergey CHUVPILO, Moscow (RU)

(21) Appl. No.: 15/320,783

(22) PCT Filed: Jun. 23, 2015
(86) PCT No.: PCT/IB2015/001428

§ 371 Date: Dec. 21, 2016

Related U.S. Application Data
(60) Provisional application No. 62/015,559, filed on Jun. 23, 2014.

(51) Int. Cl. C07K 16/28; A61K 9/00; A61K 39/395

(52) U.S. Cl. CPC C07K 16/2818; A61K 39/3955; A61K 9/0019; A61K 2039/505

(57) ABSTRACT

This invention relates to methods and compositions that are useful for the treatment of various diseases, including inflammatory diseases. The invention relates to, in part, doses and regimens for safer immunotherapy in the treatment of these diseases, including, for example, safe treatments with anti-CD28 antibodies.

### PRIORITY

This application claims priority to U.S. Provisional Patent Application No. 62/015,559, filed Jun. 23, 2014, the entire contents of which are hereby incorporated by reference in their entireties.

### FIELD OF THE INVENTION

This invention relates to methods and compositions that are useful for the treatment of various diseases, including inflammatory diseases. The invention relates to, in part, doses and regimens for safer immunotherapy in the treatment of these diseases.

### BACKGROUND

Inflammatory diseases are a large group of disorders that underlie a variety of human diseases. The immune system is often involved with inflammatory disorders. For example, rheumatoid arthritis is an autoimmune disease which is characterized by a chronic CD4+ T-cell response which has escaped normal control mechanisms. Accordingly, immunotherapy—treatment of disease by inducing, enhancing, or suppressing an immune response—has proven an attractive treatment of inflammatory diseases.

For instance, TAB08 (formerly TGN1412) is a humanized agonistic anti-CD28 monoclonal antibody which is capable of activating T-lymphocytes solely by engaging co-stimulatory receptor CD28 and can be used in immunotherapy. Ex vivo study data on human T-cells and in vivo data on relevant animal models have shown that TAB08 has profound immunomodulatory effect in diseases which are accompanied with decreased count and/or function of T-lymphocytes.

But, attendant to immunotherapies are possible health risks that come with harnessing the immune system. For example, a phase I trial of the human TGN1412 in 2006 resulted in a life-threatening cytokine release syndrome (CRS).

Therefore, there remains a need for safe and effective treatments of various diseases which exploit the immune system.

### BRIEF DESCRIPTION OF THE INVENTION

According, the present invention provides, inter alia, improved compositions, methods and uses for immunotherapy for inflammatory diseases. In one aspect, the invention pertains to a method for treating an inflammatory disease patient, comprising administering an anti-CD28 binding agent (e.g. an agent that binds to an epitope of CD28 which competes with TAB08, a monoclonal antibody or antigen-binding portion thereof with one or more CDR sequences are as in TAB08, TAB08, or an agent that comprises a light chain or heavy chain sequence of TAB08), in an amount effective to activate regulatory T (Treg) cells, without inducing substantial release of pro-inflammatory cytokines (e.g. IL-2, TNFa, and INFγ), to the patient. In some embodiments, the activation of Treg cells comprises an increase in IL-10 production. In some embodiments, the inflammatory disease is characterized by activated CD4+ T cells. In some embodiments, the inflammatory disease is characterized by an autoimmune condition. In some embodiments, the inflammatory disease is rheumatoid arthritis of various stages (e.g., Stage I, Stage II, Stage III, or Stage IV) and, optionally is in remission or active. In some embodiments, the rheumatoid arthritis is non-responsive to a corticosteroid, NSAID, COX-2 inhibitor, or biologic alone.

In various embodiments, the anti-CD28 binding agent is dosed in a manner that provides patient safety—for instance, infusion for a period of at least about 2 hours, or at least about 4 hours, or at least about 8 hours, or at least about 10 hours and administration at about 0.1 μg/kg to about 10 μg/kg of patient body weight, and a regimen of administered about once per week, about once per month, about every other month, or about one to ten times per year, or about 4 to about 12 times per year.

In various embodiments, the patient is monitored during infusion for an increase in one or more of IL-1β, IL-2, IL-4, IL-6, IL-10, IL-17, TNFa, and INFγ in circulation, to thereby assess activation of Treg cells and/or detect release of pro-inflammatory cytokines.

In various embodiments, the patient is undergoing treatment with another agent, including, for example, a corticosteroid, NSAID, COX-2 inhibitor, or biologic. In various embodiments, the patient is undergoing treatment with methotrexate and/or methylprednisolone. In some embodiments, the patient is not fully responsoive or non-responsive to methotrexate.

In another aspect, the present invention relates to a method for treating a patient having rheumatoid arthritis and undergoing treatment with a corticosteroid, comprising: administering TAB08 to said patient by slow intravenous infusion (e.g. two to twelve hours) of from 0.1 μg/kg to 7 μg/kg of patient body weight.

In a further aspect, the present invention relates to a method for treating a patient having an autoimmune disease (e.g., rheumatoid arthritis), comprising administering an anti-CD28 binding agent to the patient. In some embodiments, the patient is also undergoing treatment with methotrexate and/or methylprednisolone. In some embodiments, the patient is non-responsive to methotrexate. In some embodiments, the patient is non-responsive to methylprednisolone.

### BRIEF DESCRIPTION OF THE DRAWINGS

FIG. 1 shows induction of T-cell proliferation and cytokine production by the CD28SA TAB08 in human peripheral blood mononuclear cell (PBMCs) and sensitivity to suppression by the corticosteroid analogue methylprednisolone (MP). HD precultured (1×10⁷ cells/mL for 48 h). PBMCs were cultured in 0.2 mL AB medium at 1×10⁶ cells/mL in 96-well flat-bottom tissue culture plates. Panels A and B show T-cell proliferation was measured as incorporated ³H thymidine on day 2-3 post-stimulation with, in Panel A, the indicated concentrations of TAB08 and, in Panel B, 1 μg/mL of TAB08 in the presence of titrated concentrations of MP. Panels C-E show supernatants from cells stimulated as in (A) were collected 24 h post-stimulation and analyzed for the presence of the indicated cytokines by CBA. Panel F shows suppression of TAB08 (1 μg/mL) induced cytokine release by titrated concentrations of MP was calculated as percent of control release in the absence of MP. Data in Panels A-E are shown as mean±SD of three triplicate samples from one of three independent experiments with similar results.

FIG. 2 shows TAB08-induced expansion and proliferation of Treg cells in the absence and presence of MP. Panel A shows HD (1×10⁷ cells/mL for 48 h) precultured PBMCs were cultured in 0.6 mL AB medium for 5 days at 1×10⁶ cells/mL in 48-well flat-bottom tissue culture plates with varying concentrations of TAB08. Treg-cell (CD25^{hi}Foxp3⁺) and activated Tconv-cell (CD25^{lo}Foxp3⁻) frequencies among gated CD4⁺ T cells were then assessed by flow cytometry using a live light-scatter entrance gate. Panel B shows cells were stimulated as in (A), in the presence or absence of 0.01 mM MP. Treg-cell frequencies among CD4⁺ T cells and absolute numbers of Treg cells were determined by flow cytometry after 5 days of culture. Panel C shows HD precultured PBMCs were CFSE-labeled with and without depletion of CD25⁺ cells, and cocultured (1:1) with unlabelled HD precultured PBMCs and 1 μg/mL TAB08 for 3 days. Treg-cell versus Tconv-cell expansion in CD25-depleted and nondepleted PBMC cultures was then assessed by flow cytometry, using a live light-scatter entrance gate followed by gates for CD4⁺Foxp3⁻ (left dot plots) and CD4⁺Foxp3⁺ (right dot plots). Panels D-E show Proliferation of Treg cells versus Tconv cells after stimulation with varying concentrations of TAB08 in the presence and absence of 0.01 mM MP. Proliferation was assessed by, in Panel D, the expression of Ki67 and Panel E CFSE dilution. *denotes insufficient number of cells for evaluation. Data in Panels B, C, and E are shown as mean±SD of three samples from one experiment taken from three independent experiments with similar results. Flow cytometry data are representative of three independent experiments.

FIG. 3 shows CTLA-4 expression of TAB08-expanded Treg cells. HD precultured PBMCs were cultured as described in FIG. 2. CTLA-4 expression (intracellular and surface) by Treg (CD4⁺CD25^{hi}Foxp3⁺) versus Tconv (CD4⁺Foxp3⁻) cells was measured by flow cytometry using a live entrance gate followed by gates identifying CD4⁺Foxp3⁻ (Tconv cells) and CD4⁺Foxp3⁺ (Treg cells) after 5 days of stimulation with TAB08 at the indicated concentrations, with and without 0.01 mM MP. Data are shown as mean±SD of three parallel cultures from one of three independent experiments with similar results.

FIG. 4 shows Suppressive activity of TAB08-expanded Treg cells. (A) Treg cells were purified from HD precultured PBMCs stimulated for 5 days with TAB08 (1 or 0.1 μg/mL) in the presence or absence of 0.01 mM MP, and cocultured with purified CFSE-labeled CD4⁺ T cells, monocytes, and anti-CD3 as detailed in Materials and methods. Proliferation was determined by flow cytometry on day 3 and is shown as percentage of divided indicator cells. (B) Cell cultures from (A) were analyzed by CBA for the presence of the indicated cytokines at 24 h of culture. “0.01 mM MP” above bars indicates MP inclusion during Treg-cell expansion, not during the suppression assay. Data in (A) show single measurements of pooled cells recovered from three parallel cultures, data in (B) are shown as mean±SD of individual cytokine measurements from the supernatants of these three parallel cultures. Data are representative of three independent experiments with similar results.

FIG. 5 shows corticosteroid sensitivity of TNG1412-induced cytokine release in PBMCs from RA patients. HD precultured PBMCs from RA patients were cultured in 0.6 mL medium for 5 days at 1×106 cells/mL in 48-well flat-bottom tissue culture plates. PBMCs were stimulated with 1, 0.1, and 0.06 μg/mL TAB08, and 0.01 mM MP was used for suppression of cytokine release (n=10). A total of 0.1 mL supernatant was removed for cytokine analysis after 24 h. Each symbol represents a triplicate mean value obtained with parallel cultures set up from one blood sample. Data obtained with several (up to three) blood samples taken from the same donor at least 2 weeks apart share the same symbol. Means of all patients per condition are indicated by the horizontal bars. ****p≦0.0001, ***p≦0.0009, **p≦0.0081, *p≦0.0385 (Friedman test). IFN-γ, IL-4, IL-10, and IL-17A: no significant difference between groups.

FIG. 6 shows TAB08-expanded Treg cells from RA patients are functional suppressor cells. Panels A-E show cultures described in FIG. 5 were harvested and analyzed by flow cytometry on day 5 for Panels A and B show Treg-cell expansion, Panels C and D show proliferation (Ki67 expression) of Treg and Tconv CD4⁺ T cells, Panel E shows CTLA-4 expression (intracellular and surface) by Treg cells (CD4⁺CD25^{hi}Foxp3⁺) versus Tconv cells (CD4⁺Foxp3− cells) using the same gating strategy as in FIGS. 1-3. Panel F shows suppression of anti-CD3-stimulated proliferation of CFSE-labeled CD4⁺ T cells by TAB08 (0.06 μg/mL) expanded Treg cells from RA patient 7. Percentage of divided indicator cells was determined by flow cytometry on day 3 of culture. Experimental setup was as in FIG. 4, panel A. Panel G shows supernatants from cultures in Panel F were analyzed by CBA for the presence of cytokines after 24 h of stimulation. Panel A-E: each symbol represents a triplicate mean value obtained with parallel cultures set up from one blood sample. Data obtained with several (up to three) blood samples taken from the same donor at least 2 weeks apart share the same symbol. Means of all patients per condition are indicated by the horizontal bars. ****p≦0.0001, ***p≦0.0007, **p≦0.0062, *p≦0.0412 (Friedman test). (A and C) n=10, (B) n=8, (D) n=7, (E) n=6. (F and G) Data are shown as mean+SD of triplicate samples from one randomly chosen patient. Results are similar to three experiments performed with healthy donors.

FIG. 7 shows cytokine response of healthy volunteers to TAB08 infusion. Samples of peripheral blood were taken from healthy volunteers before and at various time points after infusion with TAB08 at the doses indicated (infusion with 0.1 μg/kg: n=6, infusion with 0.3, 0.6, 1, 1.5, 2, 3, 5, and 7 μg/kg: n=3). Infusions were performed over 4, 8, or 12 h as detailed in Materials and methods. Plasma was isolated via centrifugation and kept at −80° C. until analysis. Panel A shows serum levels of TNFa, IFN-γ, IL-2, and IL-10 of all participants. Cytokines were measured by Enhanced Sensitivity CBA. Panel B shows dose-dependent release of IL-10 at 12 h after TAB08 infusion. Data are shown as mean±SD of the IL-10 serum levels of the participants in each cohort. *p≦0.03 (unpaired t-test).

FIG. 8 shows the human patient sample of the Phase I study of Example 2. 43 healthy volunteers were screened, 10 volunteers withdrew during the screening process due to the mismatch to the criteria, 2 volunteers refused from participation on their own will, 31 volunteers received TAB08 infusion (TAB08 dose depends on the cohort number), 1 volunteer withdrew from the study on his own will, 30 volunteers completed the study in accordance with the protocol.

FIG. 9 shows pharmacokinetic curves for TAB08 concentrations in blood serum of healthy volunteer 0139 after single i.v. infusion for dose 3 μg/kg, on semi-logarithmic chart. X-coordinate—Concentration (μg/mL). Y-coordinate—Time (h).

FIG. 10 shows pharmacokinetic curves for TAB08 concentrations in blood serum of healthy volunteers after single i.v. infusion for dose 5 μg/kg, on semi-logarithmic chart. X-coordinate—Concentration (μg/mL). Y-coordinate—Time (h).

FIG. 11 shows pharmacokinetic curves for TAB08 concentrations in blood serum of healthy volunteers after single i.v. infusion for dose 7 μg/kg, on semi-logarithmic chart. X-coordinate—Concentration (μg/mL). Y-coordinate—Time (h).

FIG. 12 shows pharmacokinetic curves for TAB08 concentrations in blood serum of healthy volunteers after single i.v. infusion for doses 3, 5 and 7 μg/kg, on semi-logarithmic chart. For dose 3 μg/kg individual pharmacokinetic curve for subject 0139 is on plot; For doses 5 and 7 μg/kg—pharmacokinetic curves of mean concentrations (n=2, Mean±SE, except for 168 h−n=1). X-coordinate—Concentration (μg/mL). Y-coordinate—Time (h).

FIG. 13 shows maximum of T-regs increase in % for cohorts 5-9 (panel A), and the same expressed as a percentage (panel B).

FIG. 14 shows time to maximum T-regs subset response after TAB08 infusion.

FIG. 15 shows maximum increase of T-regs and activated T-regs in folds compared to pre-dose values.

FIG. 16 shows maximum activated T-regs increase in folds compared to pre-dose values.

FIG. 17 shows diagrams with changes of T-regs and IL-10 concentrations in volunteers blood from cohort 9.

FIG. 18 (panels A-C) show the relative number of regulatory T cells all time points in different dose cohorts.

FIG. 19 (panels A-C) show the relative number of Ki67+ regulatory T cells at all time points in different dose cohorts.

FIG. 20 (panels A-C) show cytokines concentrations at all time points in the first 5 mkg/kg dose cohort.

FIG. 21 (panels A-C) show cytokines concentration at all time points in the second 7 mkg/kg dose cohort.

FIG. 22 (panels A-C) show cytokines concentration at all time points in the third 10 mkg/kg dose cohort.

FIG. 23 (panels A-C) show TNFα concentrations at all time points in different dose cohorts.

FIG. 24 (panels A-C) shows IL-10 concentrations at all time points in different dose cohorts.

FIG. 25 (panels A-C) shows IL-2 concentrations at all time points in different dose cohorts.

FIG. 26 shows TNFα release by PBMC samples taken from responding patient NNS (panel A) and TIZ (panel B) and treated with increasing concentrations of TAB08 in the presence or absence of MP.

FIG. 27 shows proliferation of regulatory T cells in PBMC samples taken from responding patients NNS (panel A) and TIZ (panel B) and treated with increasing concentrations of TAB08 in the presence or absence of TAB08.

FIG. 28 shows levels of TAB08 in serum of RA patients treated with TAB08 at all time points in the first dose cohort (5 mkg/kg): Patient 0202 AMS (panel A), Patient 0203 MPS (panel B), and Patient 0102 GMP (panel C).

FIG. 29 shows levels of TAB08 in serum of RA patients treated with TAB08 at all time points in the second dose cohort (7 mkg/kg): Patient 0101 VIU (panel A), Patient 0204 TFY (panel B), and Patient 0205 NNS (panel C).

FIG. 30 shows levels of TAB08 in serum of RA patients treated with TAB08 at all time points in the third dose cohort (10 mkg/kg) Patient 0101 VIU.

### DETAILED DESCRIPTION OF THE INVENTION

The present invention is based, in part, on the discovery of that anti-CD28 agents can be safely and effectively administered to treat inflammatory diseases, for example TAB08 can be safely dosed to effectively treat rheumatoid arthritis.

In one aspect, the invention pertains to a method for treating an inflammatory disease patient, comprising administering an anti-CD28 binding agent, in an amount effective to activate Treg cells, without inducing substantial release of pro-inflammatory cytokines to the patient. In another aspect, the present invention provides a use of an anti-CD28 binding agent for the treatment, and/or manufacturing of a medicament for the treatment, of inflammatory diseases.

In another aspect, the invention pertains to a method for treating a patient having rheumatoid arthritis and undergoing treatment with a corticosteroid, comprising administering TAB08 to said patient by slow intravenous infusion (e.g., two to twelve hours) of from 0.1 μg/kg to 7 μg/kg of patient body weight. In another aspect, the present invention provides a use of TAB08 for the treatment, and/or manufacturing of a medicament for the treatment, of rheumatoid arthritis. TAB08 is known in the art (see, e.g., U.S. Pat. No. 7,585,960, the contents of which are hereby incorporated by reference).

In various embodiments, the inflammatory disease is characterized by activated CD4+ T cells. In various embodiments, the inflammatory disease is characterized by chronic CD4+ T-cell response which has escaped normal control mechanisms. The exact cause of accumulation of activated CD4+ T-cells is not known. In various embodiments, the inflammatory disease is characterized by the release of proinflammatory cytokines. In various embodiments, the activated CD4+ T cells (e.g. those in the synovium of patients with rheumatoid arthritis) are characterized by one or more of: T lymphocytes are found in follicular lymphoid aggregates; the cell-surface phenotype is suggestive of chronic immune activation, e.g. expression of CD45RO, CD69, and subsets of chemokine receptors; T cells are terminally differentiated, with significant telomere loss; synovial T cells are hyporesponsive to TCR ligation; synovial T cells exist in an environment favoring cell survival; there may be an imbalance of pro- and anti-inflammatory cytokines, with a predominance of macrophage products in inflamed joints; and there may be a bias towards the development of T helper (Th)1 cells.

In various embodiments, the present agents maintain T-cell homeostasis which is mediated by expansion of regulatory T-cells (Treg). Regulatory T cells are a subpopulation of T cells which modulate the immune system, maintain tolerance to self-antigens, and abrogate autoimmune diseases. Particularly, regulatory T cells maintain order in the immune system by negatively regulating the immune responses of other cells. Thus, regulatory T cells perform a critical “self-check” function to prevent excessive immune reactions. The activation of regulatory T cells is critically influenced by costimulatory and coinhibitory signals. Two major families of costimulatory molecules include the B7 and the tumor necrosis factor (TNFa) families. These molecules bind to receptors on T cells belonging to the CD28 or TNFa receptor families, respectively. The best-defined coinhibitors and their receptors belong to the B7 and CD28 families. Exemplary T cell costimulatory receptors and their ligands include, for example, CD28:B7-1/B7-2, ICOS:ICOS-L, 4-1BB:4-1BBL, OX-40:OX40-L, CD27:CD70, CD30:CD30-L, CD40:CD40-L; CD137:CD137-L, HVEM:LIGHT, GITR:GITR-L, and BTLA. Exemplary T cell coinhibitory receptors and their ligands include, for example, CTLA-4:CD80/CD86, PD1:PD-L1/PD-L2, BTLA:HVEM, TIM-3:galectin-9, B7-H3, and B7-H4.

One skilled in the art can measure changes in cell populations by measuring, for example, cell surface marker expression, gene expression, and/or secretion of proteins, cytokines, chemokines, growth factors produced by cells. These assays are well known in the art and may include, but are not limited to, flow cytometry (including, for example, fluorescent activating cell sorting (FACS)), indirect immune-fluorescence, solid phase enzyme-linked immunosorbent assay (ELISA), ELISpot assays, western blotting (including in cell western), immunofluorescent staining, microengraving (see Han Q et al. Lab Chip. 2010; 10(11):1391-1400), Quant-iT and Qubit protein assay kits, NanoOrange protein quantitation kit, CBQCA protein quantitation kits, EZQ protein quantitation kit, Click-iT reagents, Pro-Q Diamond phosphoprotein stain, Pro-Q glycoprotein stain kits, peptide and protein sequencing, N-terminal amino acid analysis (LifeScience Technologies, Grand Island, N.Y.), chemiluminescence or colorimetric based ELISA cytokine Arrays (Signosis) Intracellular Cytokine Staining (ICS), BD Phosflow™ and BD™ Cytometric Bead Arrays (BD Sciences, San Jose, Calif.); RT-PCR (RT2 Profiler™ Human Common Cytokine PCR Arrays (Cat #PAHS-021) ((SABiosciences/QIAGEN)); CyTOF Mass Cytometer (DVS Sciences, Sunnyvale Calif.); Mass Spectrometry; Microplate capture and detection assay (Thermo Scientific, Rockland, Ill.), Multiplex Technologies (for example Luminex, Austin, Tex.); FlowCellect™ T Cell Activation Kit (EMD Millipore); Surface Plasmon Resonance (SPR)-based technologies (for example Biacore, GE Healthcare Life Sciences, Uppsala, Sweden); CD4⁺ Effector Memory T Cell Isolation Kit and CD8⁺CD45RA⁺ Effector T Cell Isolation Kit (Miltenyi Biotec Inc., CA); The EasySep™ Human T Cell Enrichment Kit (StemCells, Inc., Vancouver, Canada); HumanTh1/Th2/Th17 Phenotyping Kit (BD Biosciences, CA); MultiTox Multiplex Assays, CellTitre-Glo Assays, CellTiter-Fluor Cell Viability Assay (Promega, Madison, Wis.). See also, Current Protocols in Immunology (2004) sections 3.12.1-3.12.20 by John Wiley & Sons, Inc., or Current Protocols in Immunology (2013) or by John Wiley & Sons, Inc, the contents of which are herein incorporated by reference in their entireties.

In various embodiments, the present invention relates to treatment and/or manufacturing of a medicament for inflammatory diseases, including inflammatory abnormalities and autoimmune diseases or disorders (i.e. those characterized by an autoimmune condition).

In some embodiments, inflammatory abnormalities include a large group of disorders that are often tied to the immune system is often involved with inflammatory disorders, demonstrated in both allergic reactions and some myopathies, with many immune system disorders resulting in abnormal inflammation. Inflammatory abnormalities include, but are not limited to, acne vulgaris, asthma, autoimmune diseases, celiac disease, chronic prostatitis, glomerulonephritis, hypersensitivities, inflammatory bowel diseases, pelvic inflammatory disease, reperfusion injury, rheumatoid arthritis, sarcoidosis, transplant rejection, vasculitis, and interstitial cystitis.

In some embodiments, inflammatory diseases include autoinflammatory diseases or disorders, which are diseases or disorders characterized by intense episodes of inflammation that result in such symptoms as fever, rash, or joint swelling. These diseases also carry the risk of amyloidosis, a potentially fatal build up of a blood protein in vital organs. The present invention pertains to several different types of autoinflammatory diseases, including but not limited to familial Mediterranean fever (FMF) suffer from recurring bouts of fever, most commonly with severe abdominal pain due to inflammation of the abdominal cavity (peritonitis). Attacks can also include arthritis (painful, swollen joints), chest pain from inflammation of the lung cavity (pleurisy), and skin rashes. In various embodiments, including in the context of FMF, the present anti-CD28 agents may supplement or supplant treatment with colchicine. Another autoinflammatory disease that is encompassed in the present invention is Neonatal Onset Multisystem Inflammatory Disease (NOMID), also known as chronic infantile neurologic cutaneous articular (CINCA) syndrome, which affects numerous organs and body systems, including the skin, joints, eyes, and central nervous system. For most children, the first sign of the disease is a rash that develops within the first 6 weeks of life; other problems, including fever, meningitis, joint damage, vision and hearing loss, and mental retardation, can follow and the disease is progressive and often fatal. As much as 20 percent of children with NOMID don't survive to adulthood. In various embodiments, including in the context of NOMID, the present anti-CD28 agents may supplement or supplant treatment with anakinra. Yet another autoinflammatory disease that is encompassed in the present invention is Tumor Necrosis Factor (TNFa) Receptor-Associated Periodic Syndrome (TRAPS), formerly known as familial Hibernian fever, is characterized by long, dramatic, episodes of high fever; severe pain in the abdomen, chest, or joints; skin rash; and inflammation in or around the eyes. The age of onset varies from early childhood to adulthood, and the disease appears to affect men and women equally. The earliest cases of TRAPS were reported in individuals of Irish-Scottish descent, but the disease has since been found in nearly all ethnic groups. In various embodiments, including in the context of TRAPS, the present anti-CD28 agents may supplement or supplant treatment with TNFa inhibitors (including but not limited to Infliximab (REMICADE), Etanercept (ENBREL), Adalimumab (HUMIRA), Certolizumab (CIMZIA), Golimumab (SIMPONI), curcumin catechins, cannabis, and Echinacea purpurea). Still another autoinflammatory disease that is encompassed in the present invention is Deficiency of the Interleukin-1 Receptor Antagonist (DIRA), in which subjects (including children) with the disorder display a constellation of serious and potentially fatal symptoms that include swelling of bone tissue; bone pain and deformity; inflammation of the periosteum; and a rash that can span from small individual pustules to extensive pustulosis that covers most of the patient's body. In various embodiments, including in the context of DIRA, the present anti-CD28 agents may supplement or supplant treatment with anakinra and/or a synthetic form of human IL-1Ra. Further Behcet's Disease is an autoinflammatory disease that is encompassed in the present invention. Behcet's disease causes canker sores or ulcers in the mouth and on the genitals and inflammation in parts of the eye. In some people, the disease also results in arthritis, skin problems, and inflammation of the digestive tract, brain, and spinal cord. In various embodiments, including in the context of Behcet's Disease, the present anti-CD28 agents may supplement or supplant treatment with corticosteroids and immunosuppressive drugs.

In some embodiments, the disease to be treated is one characterized by a defective and/or deficiency in one or more of the following illustrative manners to terminate inflammation: short half-life of inflammatory mediators; production and release of transforming growth factor (TGF) beta (e.g. from macrophages); production and release of interleukin 10 (IL-10); production of anti-inflammatory lipoxins; downregulation of pro-inflammatory molecules, such as, for example, leukotrienes; upregulation of anti-inflammatory molecules such as the interleukin 1 receptor antagonist or the soluble tumor necrosis factor receptor (TNFaR); apoptosis of pro-inflammatory cells; desensitization of receptors; increased survival of cells in regions of inflammation due to, for example, their interaction with the extracellular matrix (ECM); downregulation of receptor activity by high concentrations of ligands; cleavage of chemokines by, for example, matrix metalloproteinases (MMPs) might lead to production of anti-inflammatory factors; and production of resolvins, protectins, or maresins.

In various embodiments, the present invention pertains to the treatment of, and/or manufacture of a medicament for the treatment of, rheumatoid arthritis (RA); psoriatic arthritis; psoriasis; inflammatory bowel syndrome (IBD); Crohn's disease; ulcerative colitis; multiple sclerosis (MS); flu, including pandemic flu; respiratory disorders, for example those caused by viruses, such as respiratory syncytial virus (RSV); cystic fibrosis; herpes, including genital herpes; asthma and allergies; sepsis and septic shock; bacterial pneumonia; bacterial meningitis; dengue hemorrhagic fever; diabetes Type I; endometriosis; prostatitis; uveitis; uterine ripening; and age-related macular degeneration.

In some embodiments, the autoimmune condition involves hypersensitivity Type II, Ill, or IV. Hypersensitivity refers to excessive, undesirable reactions produced by the normal immune system. Hypersensitivity reactions require a pre-sensitized (immune) state of the host and can be divided into four types: type I, type II, type III and type IV, based on the mechanisms involved and time taken for the reaction. In some embodiments, the autoimmune condition of the present invention may involve more than one type of reaction.

Type I hypersensitivity is also known as immediate or anaphylactic hypersensitivity. The reaction may involve skin (urticaria and eczema), eyes (conjunctivitis), nasopharynx (rhinorrhea, rhinitis), bronchopulmonary tissues (asthma) and gastrointestinal tract (gastroenteritis). The reaction may cause a range of symptoms from minor inconvenience to death. The reaction usually takes 15-30 minutes from the time of exposure to the antigen, although sometimes it may have a delayed onset (10-12 hours). Immediate hypersensitivity is mediated by IgE. The primary cellular component in this hypersensitivity is the mast cell or basophil. The reaction is amplified and/or modified by platelets, neutrophils and eosinophils. A biopsy of the reaction site demonstrates mainly mast cells and eosinophils. The mechanism of reaction involves preferential production of IgE, in response to certain antigens (allergens). IgE has very high affinity for its receptor on mast cells and basophils. A subsequent exposure to the same allergen cross links the cell-bound IgE and triggers the release of various pharmacologically active substances. Cross-linking of IgE Fc-receptor is important in mast cell triggering. Mast cell degranulation is preceded by increased Ca²⁺ influx, which is a crucial process; ionophores which increase cytoplasmic Ca²⁺ also promote degranulation, whereas, agents which deplete cytoplasmic Ca³⁺ suppress degranulation. The agents released from mast cells may include histamine, tryptase, kininogenase, ECF-A (tetrapeptides) The reaction is amplified by PAF (platelet activation factor) which causes platelet aggregation and release of histamine, heparin and vasoactive amines. Eosinophil chemotactic factor of anaphylaxis (ECF-A) and neutrophil chemotactic factors attract eosinophils and neutrophils, respectively, which release various hydrolytic enzymes that cause necrosis. Eosinophils may also control the local reaction by releasing arylsulphatase, histaminase, phospholipase-D and prostaglandin-E, although this role of eosinophils is now in question.

In some embodiments, the present treatments with anti-CD28 agents can be paired with diagnostic tests for immediate hypersensitivity including skin (prick and intradermal) tests and measurement of total IgE and specific IgE antibodies against the suspected allergens. Total IgE and specific IgE antibodies may be measured by a modification of enzyme immunoassay (ELISA). Increased IgE levels are indicative of an atopic condition, although IgE may be elevated in some non-atopic diseases (e.g., myelomas, helminthic infection, etc.).

In various embodiments, the anti-CD28 agents may be administered as adjuvant or neoadjuvants to various agents described herein. For example, antihistamines, which, without wishing to be bound by theory, block histamine receptors; chromolyn sodium, which, without wishing to be bound by theory, inhibits mast cell degranulation (e.g. by inhibiting Ca²⁺ influx). Also, leukotriene receptor blockers (Singulair, Accolate) or inhibitors of the cyclooxygenase pathway (Zileutoin) may be used in combination with the present anti-CD28 agents. Further, bronchodilators (inhalants) such as isoproterenol derivatives (Terbutaline, Albuterol) and hyposensitization (immunotherapy or desensitization) are other combination treatment modalities. In all examples of the various agents described herein, the present invention contemplates administering an anti-CD28 agent to a patient undergoing treatment with one or more of the various agents.

In various embodiments, the autoimmune condition involves Type II hypersensitivity, also known as cytotoxic hypersensitivity. The antigens are normally endogenous, although exogenous chemicals (haptens) which can attach to cell membranes can also lead to type II hypersensitivity. Drug-induced hemolytic anemia, granulocytopenia and thrombocytopenia are such examples. The reaction time is minutes to hours. Type II hypersensitivity is primarily mediated by antibodies of the IgM or IgG classes and complement but phagocytes and K cells may also play a role (ADCC). In some embodiments, the present treatments with anti-CD28 agents can be paired with diagnostic tests including detection of circulating antibody against the tissues involved and the presence of antibody and complement in the lesion (biopsy) by immunofluorescence. The staining pattern is normally smooth and linear, such as that seen in Goodpasture's nephritis (renal and lung basement membrane) and pemphigus (skin intercellular protein, desmosome). In various embodiments, the anti-CD28 agents may be administered as adjuvant or neoadjuvants to various agents described herein. For example, anti-inflammatory and immunosuppressive agents.

In various embodiments, the autoimmune condition involves Type III hypersensitivity, also known as cytotoxic hypersensitivity or immune complex hypersensitivity. The reaction may be general (e.g., serum sickness) or may involve individual organs including skin (e.g., systemic lupus erythematosus, Arthus reaction), kidneys (e.g., lupus nephritis), lungs (e.g., aspergillosis), blood vessels (e.g., polyarteritis), joints (e.g., rheumatoid arthritis) or other organs. This reaction may be the pathogenic mechanism of diseases caused by many microorganisms. The reaction may take 3-10 hours after exposure to the antigen (as in Arthus reaction). It is mediated by soluble immune complexes. They are mostly of the IgG class, although IgM may also be involved. The antigen may be exogenous (chronic bacterial, viral or parasitic infections), or endogenous (non-organ specific autoimmunity: e.g., systemic lupus erythematosus, SLE). The antigen is soluble and not attached to the organ involved. Primary components are soluble immune complexes and complement (C3a, 4a and 5a). The damage is caused by platelets and neutrophils. The lesion contains primarily neutrophils and deposits of immune complexes and complement. Macrophages infiltrating in later stages may be involved in the healing process. In some embodiments, the present treatments with anti-CD28 agents can be paired with diagnostic tests including examination of tissue biopsies for deposits of Ig and complement by immunofluorescence. The immunofluorescent staining in type III hypersensitivity is granular (as opposed to linear in type II such as seen in Goodpasture's syndrome). The presence of immune complexes in serum and depletion in the level of complement are also diagnostic. Polyethylene glycol-mediated turbidity (nephelometry), binding of C1q and Raji cell test are utilized to detect immune complexes. In various embodiments, the anti-CD28 agents may be administered as adjuvant or neoadjuvants to various agents described herein. For example, anti-inflammatory agents may be used.

In various embodiments, the autoimmune condition involves Type IV hypersensitivity, also known as cytotoxic hypersensitivity or cell mediated or delayed type hypersensitivity. An example of this hypersensitivity is tuberculin (Montoux) reaction which peaks 48 hours after the injection of antigen (PPD or old tuberculin). The lesion is characterized by induration and erythema. Type IV hypersensitivity is involved in the pathogenesis of many autoimmune and infectious diseases (e.g. tuberculosis, leprosy, blastomycosis, histoplasmosis, toxoplasmosis, leishmaniasis, etc.) and granulomas due to infections and foreign antigens. Another form of delayed hypersensitivity is contact dermatitis (poison ivy, chemicals, heavy metals, etc.) in which the lesions are more papular. Mechanisms of damage in delayed hypersensitivity include, without wishing to be bound by theory, T lymphocytes and monocytes and/or macrophages. Cytotoxic T cells (Tc) cause direct damage whereas helper T (TH1) cells secrete cytokines which activate cytotoxic T cells and recruit and activate monocytes and macrophages, which, without wishing to be bound by theory, cause the bulk of the damage). The delayed hypersensitivity lesions mainly contain monocytes and a few T cells. Lymphokines involved in delayed hypersensitivity reaction include monocyte chemotactic factor, interleukin-2, interferon-gamma, TNFa alpha/beta, etc. In some embodiments, the present treatments with anti-CD28 agents can be paired with diagnostic tests including delayed cutaneous reaction (e.g. Montoux test) and patch test (for contact dermatitis). In vitro tests for delayed hypersensitivity include mitogenic response, lympho-cytotoxicity and IL-2 production. In various embodiments, the anti-CD28 agents may be administered as adjuvant or neoadjuvants to various agents described herein. For example, corticosteroids and other immunosuppressive agents may be used.

In various embodiments, the inflammatory disease is rheumatoid arthritis (RA), an autoimmune disease that results in a chronic, systemic inflammatory disorder that may affect many tissues and organs, but principally attacks flexible (synovial) joints. In some embodiments, the present invention pertains to the treatment of RA with anti-CD 28 agents, including at specific doses and regimens described herein. In some embodiments, the present invention pertains to the use of anti-CD 28 agents in the treatment of RA, including at specific doses and regimens described herein. In some embodiments, the present invention pertains to the use of anti-CD 28 agents in manufacture of a medicament for the treatment of RA, including at specific doses and regimens described herein. RA is often a disabling and painful condition, and may result in substantial loss of functioning and mobility. RA is often characterized by an inflammatory response of the synovium secondary to swelling (turgescence) of synovial cells, excess synovial fluid, and the development of pannus in the synovium. The pathology of the disease process often leads to the destruction of articular cartilage and ankylosis of the joints. RA can also produce diffuse inflammation in the lungs, the membrane around the heart (pericardium), the membranes of the lung (pleura), and white of the eye (sclera), and also nodular lesions, most common in subcutaneous tissue.

In various embodiments, the RA is Stage I, Stage II, Stage III, or Stage IV. Early stage RA (stage I) is characterized by synovitis, or an inflammation of the synovial membrane, causing swelling of involved joints and pain upon motion. During this stage, there is a high cell count in synovial fluid as immune cells migrate to the site of inflammation. However, there is generally no x-ray evidence of joint destruction, with the exception of swelling of soft tissues and possibly evidence of some bone erosion. In various embodiments, the anti-CD28 agents of the present invention (including but not limited to TAB08) are used to treat Stage I RA. In moderate RA, stage II, there is a spread of inflammation in synovial tissue, affecting joint cavity space across joint cartilage. This inflammation will gradually result in a destruction of cartilage, accompanied by a narrowing of the joint. In various embodiments, the anti-CD28 agents of the present invention (including but not limited to TAB08) are used to treat Stage II RA. Severe RA, stage III, is marked by formation of pannus in the synovium. Loss of joint cartilage exposes bone beneath the cartilage. These changes will become evident on x-ray, along with erosions around the margins of the joint. Joint deformities may also become evident. In various embodiments, the anti-CD28 agents of the present invention (including but not limited to TAB08) are used to treat Stage III RA. Stage IV is called terminal or end stage RA. The inflammatory process has subsided and formation of fibrous tissue and/or fusing of bone results in ceased joint function. This stage may be associated with formation of subcutaneous nodules. In various embodiments, the anti-CD28 agents of the present invention (including but not limited to TAB08) are used to treat Stage IV RA.

In some embodiments, the anti-CD28 agents of the present invention (including but not limited to TAB08) are used to treat RA that is characterized by a disease activity score (DAS) of from 0-10. In some embodiments the DAS is 0. or 1, or 2, or 3, or 4, or 5, or 6, or 7, or 8, or 9, or 10. The DAS may measure 28 joints (DAS28). The joints included in DAS28 are (bilaterally): proximal interphalangeal joints (10 joints), metacarpophalangeal joints (10), wrists (2), elbows (2), shoulders (2) and knees (2). When looking at these joints, both the number of joints with tenderness upon touching (TEN28) and swelling (SW28) are counted. In addition, the erythrocyte sedimentation rate (ESR) is measured. Also, the patient makes a subjective assessment (SA) of disease activity during the preceding 7 days on a scale between 0 and 100, where 0 is “no activity” and 100 is “highest activity possible”. With these parameters, DAS28 is calculated as:

DAS28=0.56×\sqrt{TEN28}+0.28×\sqrt{SW28}+0.70×ln(ESR)+0.014×SA

From this, the disease activity of the patient may be classified in the following illustrative manner:

| Current | | DAS28 decrease from initial value | | |
|---|---|---|---|---|
| DAS28 | | ≧1.2 | >0.6 but ≦1.2 | ≦0.6 |
| ≦3.2 | Inactive | Good improvement | Moderate improvement | No improvement |
| ≧3.2 but ≦5.1 | Moderate | Moderate improvement | Moderate improvement | No improvement |
| >5.1 | Very active | Moderate improvement | No improvement | No improvement |

In some embodiments, the anti-CD28 agents of the present invention (including but not limited to TAB08) are used to treat RA that is characterized by the Ritchie Articular Index (RAD; a 44-joint swollen joint count, erythrocyte sedimentation rate (ESR), and a general health assessment on a visual analog scale. In various embodiments, the RAI ranges from 0 to 78, the 44-joint swollen joint count ranges from 0 to 44, ESR may range from 0 to 150, and General Health (GH) ranges from 0 to 100. In some embodiments, the anti-CD28 agents of the present invention (including but not limited to TAB08) are used to treat RA in the initiation phase, amplification phase, or chronic inflammatory phase.

In some embodiments, the anti-CD28 agents of the present invention (including but not limited to TAB08) are used to treat RA which is in remission. In some embodiments, the anti-CD28 agents of the present invention (including but not limited to TAB08) are used to treat RA which is active. In some embodiments, the anti-CD28 agents of the present invention (including but not limited to TAB08) are used to treat a patient with a high likelihood of remission, including one or more low disease activity (e.g. DAS <about 4), favorable health assessment (e.g. health assessment questionnaire score <about 1.25), low level of joint tenderness (e.g. RAI <about 17), C reactive protein of <about 14.5 mg/l, and low morning stiffness <about 60 minutes.

Metrics for disease state are found in Arthritis & Rheumatism (Arthritis Care & Research) Vol. 49, No. 5S, Oct. 15, 2003, pp S214-S224 and Arthritis Care & Research Vol. 64, No. 5, May 2012, pp 640-647, the contents of which are hereby incorporated by reference in their entireties).

In some embodiments, the anti-CD28 agents of the present invention (including but not limited to TAB08) are used to treat RA which is non-responsive to a corticosteroid, NSAID, COX-2 inhibitor, or biologic alone.

As used herein “patient” is interchangeable with “subject” or “animal.” In some embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In some embodiments, the subject and/or animal may comprise fluorescently-tagged cells (with e.g. GFP). In some embodiments, the subject and/or animal is a transgenic animal comprising a fluorescent cell. In some embodiments, the subject and/or animal is a human. In some embodiments, the human is an at risk group—including women of about 30 to about 50 years of age (e.g. about 30, about 25, about 40, about 45, or about 50). In some embodiments, the human patient may be from a group having a higher epidemiological prevalence for RA—e.g. Native American groups.

In various embodiments, the RA to be treated by the present agents may be detected in various manners. In some embodiments, the invention provides for detecting RA as described herein and administering the agents described herein or administering the agents described herein to a patient that has been subjected to any of the detection methods described herein. In some embodiments, RA is detected with various imaging methods, including X-rays of the hands and feet (e.g. of people with a polyarthritis). In RA, there may be no changes in the early stages of the disease, or the x-ray may demonstrate juxta-articular osteopenia, soft tissue swelling and loss of joint space. As the disease advances, there may be bony erosions and subluxation. X-rays of other joints may be taken if symptoms of pain or swelling occur in those joints. In some embodiments, magnetic resonance imaging (MRI) and ultrasound may be used to assess the RA. Also, high-frequency transducers (10 MHz or higher) of ultrasound images may be used; these images can depict 20% more erosions than conventional radiography. Color Doppler and power Doppler ultrasound, which show vascular signals of active synovitis depending on the degree of inflammation, are useful in assessing synovial inflammation.

Further, various blood tests may be used in the present invention. For example, testing for the presence of rheumatoid factor (RF, a non-specific antibody). A negative RF does not rule out RA; rather, the arthritis is called seronegative, as in about 15% of patients. During the first year of illness, rheumatoid factor is more likely to be negative with some individuals converting to seropositive status over time. Additionally, anti-citrullinated protein antibodies (ACPAs) or anti-CCP (cyclic citrullinated peptide), and anti-MCV (e.g. antibodies against mutated citrullinated Vimentin) agents may be used. Also, several other blood tests are usually done to allow for other causes of arthritis, such as lupus erythematosus. The erythrocyte sedimentation rate (ESR), C-reactive protein, full blood count, renal function, liver enzymes and other immunological tests (e.g., antinuclear antibody/ANA) are all performed at this stage. Elevated ferritin levels can reveal hemochromatosis, a mimic of RA, or be a sign of Still's disease, a seronegative, usually juvenile, variant of rheumatoid arthritis. In some embodiments, 14-3-3 eta Protein testing for RA may be used (see, e.g., Maksymowych, et al. BioTechniques 2012 Protocol Guide Development of a Highly Specific 14-3-3 eta (η) ELISA for Rheumatoid Arthritis, the contents of which are hereby incorporated by reference).

In some embodiments, the present anti-CD28 find use in companion to certain diagnostics outlined herein. In various embodiments, the diagnostics may employ standard detection methods as known in the art and described herein (including but not limited to ELISA, latex agglutination/immunoturbidimetry, Westergren or modified Westergren method, nephelometry, etc.). In some embodiments, the present anti-CD28 find use in patients that have been screened one or more of the following diagnostic tests: Rheumatoid Arthritis Diagnostic Panel IdentRA™ with 14-3-3 eta (QUEST, e.g. using an ELISA or latex agglutination/immunoturbidimetry platform), Cyclic Citrullinated Peptide (CCP) Antibody (IgG, e.g. ELISA)), Rheumatoid Factor (RF, e.g. using latex agglutination/immunoturbidimetry), Rheumatoid Factor (IgA) (e.g. ELISA) which may provide added specificity when used in combination with other RF or anti-CCP assays and may help predict severity of disease course, Rheumatoid Factor (IgG) (e.g. ELISA) which provides added specificity when used in combination with other RF or anti-CCP assays, Rheumatoid Factor (IgA, IgG, IgM) (e.g. ELISA), which, by detecting all 3 isotypes, may improve the specificity and predictive value, Erythrocyte Sedimentation Rate (ESR) (e.g. with a Westergren or modified Westergren method, such as JAMA. 1940; 114(9):756, the contents of which are hereby incorporated by reference) and C-Reactive Protein (CRP) (e.g. nephelometry).

In some embodiments, the rheumatoid arthritis is non-responsive to a corticosteroid, NSAID, COX-2 inhibitor, or biologic alone.

In various embodiments, the corticosteroid is one or more of dexamethasone (e.g. DECADRON), methylprednisolone (e.g. DEPO-MEDROL, MEDROL), prednisolone, prednisone, triamcinolone (e.g. ARISTOSPAN).

In various embodiments, the NSAID is one or more of aspirin (e.g. BUFFERIN, BAYER), celecoxib (e.g. CELEBREX),diclofenac (e.g. CATAFLAM, VOLTAREN), diflunisal (e.g. DOLOBID), etodolac (e.g. LODINE), fenoprofen (e.g. NALFON), flurbiprofen (e.g. ANSAID), ibuprofen (e.g. ADVIL, MOTRIN), indomethacin (e.g. INDOCIN), ketoprofen (e.g. ORUVAIL, ORUDIS), ketorolac (e.g. TORADOL), meloxicam (e.g. MOBIC), nabumetone (e.g. RELAFEN), naproxen (e.g. ALEVE, ANAPROX, NAPRELAN, NAPROSYN), oxaprozin (e.g. DAYPRO), piroxicam (e.g. FELDENE), salsalate (e.g. AMIGESIC), sulindac (e.g. CLINORIL), tolmetin (e.g. TOLECTIN). In various embodiments, the NSAID is one or more of diclofenac (e.g. CATAFLAM, VOLTAREN, ARTHROTEC (combination with misoprostol)); diflunisal (e.g. DOLOBID); etodolac (e.g. LODINE, LODINE XL); fenoprofen (e.g. NALFON, NALFON 200); flurbiprofen (e.g. ANSAID); ibuprofen (e.g. MOTRIN, MOTRIN IB, MOTRIN MIGRAINE PAIN, ADVIL, ADVIL MIGRAINE LIQUI-GELS, IBU-TAB 200, MEDIPREN, CAP-PROFEN, TAB-PROFEN, PROFEN, IBUPROHM, CHILDREN'S ELIXSURE, VICOPROFEN (combination with hydrocodone), COMBUNOX (combination with oxycodone); indomethacin (e.g. INDOCIN, INDOCIN SR, INDO-LEMMON, INDOMETHEGAN); ketoprofen (e.g. ORUVAIL, ORUDIS, ACTRON); ketorolac (e.g. TORADOL); mefenamic acid (e.g. PONSTEL); meloxicam (e.g. MOBIC); nabumetone (e.g. RELAFEN); naproxen (e.g. ALEVE, NAPROSYN, ANAPROX, ANAPROX DS, EC-NAPROXYN, NAPRELAN, NAPRAPAC (copackaged with lansoprazole)); oxaprozin (e.g. DAYPRO); piroxicam (e.g. FELDENE); sulindac (e.g. CLINORIL); and tolmetin (e.g. TOLECTIN, TOLECTIN DS, TOLECTIN 600)

In various embodiments, the COX-2 inhibitor is one or more of Celecoxib (e.g. CELEBREX), Valdecoxib (e.g. BEXTRA), and Rofecoxib (e.g. VIOXX).

In various embodiments, the biologic is one or more of tumor necrosis factor alpha (TNFα) blockers such as infliximab; interleukin 1 blockers such as anakinra, monoclonal antibodies against B cells such as rituximab, T cell costimulation blocker such as abatacept among others. In various embodiments, the biologic is one or more of ACTEMRA, CIMZIA, ENBREL, HUMIRA, KINERET, ORENCIA, REMICADE, RITUXAN, ANDSIMPONI. In some embodiments, these biologics are used in combination with methotrexate or leflunomide. In some embodiments, rheumatoid arthritis is non-responsive to these biologics when used in combination with methotrexate or leflunomide

In various embodiments, the rheumatoid arthritis is non-responsive to disease-modifying antirheumatic drugs (DMARDs), including one or more of: methotrexate, hydroxychloroquine (PLAQUENIL), leflunomide (ARAVA), cyclosporine (NEORAL), sulfasalzine (AZULFIDINE), methotrexate (RHEUMATREX, TREXALL), azathioprine (IMURAN), cyclophosphamide (CYTOXAN), sodium aurothiomalate (Gold) and cyclosporin.

In various embodiments, the present invention provides uses and methods of treatment with anti-CD28 agents (including by way of non-limiting example TAB08) in combination with any of the agents described herein. In various embodiments, the present invention provides uses and methods of treatment with anti-CD28 agents (including by way of non-limiting example TAB08) in a patient who is undergoing treatment with a corticosteroid, NSAID, COX-2 inhibitor, or biologic as described herein. In some embodiments, the patient is undergoing treatment with an NSAID selected from Aspirin, Celecoxib, Diclofenac, Diflunisal, Etodolac, Fenoprofen, Flurbiprofen, Ibuprofen, Indomethacin, Ketoprofen, Ketorolac, Meloxicam, Nabumetone, Naproxen, Oxaprozin, Piroxicam, Salsalate, Sulindac, and Tolmetin. In some embodiments, the patient is undergoing treatment with a corticosteroid selected from dexamethasone, methylprednisolone, triamcinolone, cortisone, and prednisone. In some embodiments, the patient is undergoing treatment with a biologic selected from Actemra, Cimzia, Enbrel, Humira, Kineret, Orencia, Remicade, Rituxan, and Simponi.

In various embodiments, the present anti-CD28 agents (including by way of non-limiting example TAB08) are useful as adjuvant therapies. Adjuvant therapy is treatment that is given in addition to a primary or main treatment. In various embodiments, the inflammatory disease (e.g. RA) is aggressive and the patient is undergoing one or more primary or main treatments as described herein. The present anti-CD28 agents (including by way of non-limiting example TAB08) may be administered in conjunction with a primary or main treatment in the context of an aggressive inflammatory disease.

Also, in various embodiments, the present anti-CD28 agents (including by way of non-limiting example TAB08) are useful as neoadjuvant therapies. Neoadjuvant therapy refers to therapy that is given to before primary or main treatment, often to prepare a patient for the primary or main treatment. For example, in some embodiments, the inflammatory disease (e.g. RA) being treated is aggressive and therefore treatment with the present agents will lessen the severity of the disease and make it more amenable to a primary or main treatment.

The present anti-CD28 agents (including by way of non-limiting example TAB08) find use in a variety of combination therapies (including as sequential or simultaneous co-administration and/or as co-formulation), including the adjuvant and neoadjuvant approaches described herein. Combinations may be with any of the agents described herein and may include anti-CD28 agents (including by way of non-limiting example TAB08) with 1, or 2, or 3, or 4 other agents described herein. Standard regimens of the additional agents may be used in conjunction with the doses and regiments of the present anti-CD28 agents (including by way of non-limiting example TAB08) as described herein. For example, the present anti-CD28 agents (including by way of non-limiting example TAB08) may be added to the regimen for infliximab (REMICADE)(e.g. initially administered via intravenous infusion (IV) at a dose of about 3-5 mg/kg (according to body weight) at about weeks 0, 2, and 6 with maintenance: IV infusions about every 4-8 weeks. Dose may be increased to about 5-10 mg/kg for about 2-3 weeks); etanercept (ENBREL) (e.g. initially: about 50 mg once a week or about 25 mg twice a week as a self-administered subcutaneous injection with maintenance of the same dose for about 1-2 weeks); adalimumab (HUMIRA) (e.g. initially: about 40 mg every other week as a self-administered subcutaneous injection with maintenance of the same dose for about 2-3 weeks); certolizumab (CIMZIA) (e.g. initially: about 400 mg on about week 0, 2 and 4 (administered as 2 injections of about 200 mg each) as a self-administered subcutaneous injection with maintenance of about 200 mg every other week); and golimumab (SIMPONI) (e.g. initially: about 50 mg once per month as a self-administered subcutaneous injection with maintenance of the same).

In some embodiments, administering an effective amount of the present anti-CD28 agents (including by way of non-limiting example TAB08) increases the ability of a patient to receive a greater dose or longer duration of therapy for the various inflammatory diseases described herein, including RA.

In various embodiments, the present invention provides a method for treating a patient having an autoimmune disease, comprising administering the present anti-CD28 agents to the patient. In some embodiment, the anti-CD28 agent is administered to the patient in combination with a disease-modifying antirheumatic drug (DMARD), including one or more of methotrexate, hydroxychloroquine (e.g. PLAQUENIL), leflunomide (e.g. ARAVA), cyclosporine (e.g. NEORAL), sulfasalzine (e.g. AZULFIDINE), methotrexate (e.g. RHEUMATREX, TREXALL), azathioprine (e.g. IMURAN), cyclophosphamide (e.g. CYTOXAN), sodium aurothiomalate (Gold) and cyclosporin. In some embodiments, the anti-CD28 agent is administered to the patient in combination with a corticosteroid, including one or more of dexamethasone (e.g. DECADRON), methylprednisolone (e.g. DEPO-MEDROL, MEDROL), prednisolone, prednisone, and triamcinolone (e.g. ARISTOSPAN). In some embodiments, the anti-CD28 agent is administered to the patient in combination with the DMARD (e.g., methotrexate) and/or the corticosteroid (e.g., methylprednisolone).

The anti-CD28 agent may be administered sequentially or as simultaneous co-administration and/or as co-formulation with the DMARD (e.g., methotrexate) and/or the corticosteroid (e.g., methylprednisolone). For example, the anti-CD28 agent may be administered simultaneously with the DMARD (e.g., methotrexate) and/or the corticosteroid (e.g., methylprednisolone). In another example, the anti-CD28 agent is administered after administration of the DMARD (e.g., methotrexate) and/or the corticosteroid (e.g., methylprednisolone). In a further example, the anti-CD28 agent is administered before administration of the DMARD (e.g., methotrexate) and/or the corticosteroid (e.g., methylprednisolone). In various embodiments, the patient is undergoing treatment with the DMARD (e.g., methotrexate) and/or the corticosteroid (e.g., methylprednisolone). In various embodiments, the patient is non-responsive to the DMARD (e.g., methotrexate) and/or the corticosteroid (e.g., methylprednisolone).

In various embodiments, administration of the CD-28 agent and/or DMARD (e.g., methotrexate) and/or the corticosteroid (e.g., methylprednisolone) is performed subcutaneously, intravenously, intrathecally, orally or intramuscularly. In an embodiment, administration is performed orally. In another embodiment, administration is performed intramuscularly. In a further embodiment, administration is performed intravenous. For example, the CD-28 agent may be administered intravenously. In various embodiments, the CD-28 agent is administered intravenously at a dosage of about 1 μg/kg to about 10 μg/kg of the patient body weight. For example, the CD-28 agent is administered intravenously at a dosage of about 1 μg/kg, 1.5 μg/kg, 2 μg/kg, 2.5 μg/kg, 3 μg/kg, 3.5 μg/kg, 4 μg/kg, 4.5 μg/kg, 5 μg/kg, 5.5 μg/kg, 6 μg/kg, 6.5 μg/kg, 7 μg/kg, 7.5 μg/kg, 8 μg/kg, 8.5 μg/kg, 9 μg/kg, 9.5 μg/kg, or about 10 μg/kg of patient body weight, inclusive of all values and ranges therebetween. In another example, the corticosteroid (e.g., methylprednisolone) is administered orally or intramuscularly. In various embodiments, the corticosteroid (e.g., methylprednisolone) is administered at a dosage of about 20 mg to about 200 mg. For example, the corticosteroid (e.g., methylprednisolone) is administered at a dosage of about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg, inclusive of all values and ranges therebetween.

In various embodiments, the present invention provides a method for treating a patient having rheumatoid arthritis, comprising administering the present anti-CD28 agents to the patient. In some embodiment, the anti-CD28 agent is administered to the patient in combination with a disease-modifying antirheumatic drug (DMARD), including one or more of methotrexate, hydroxychloroquine (PLAQUENIL), leflunomide (ARAVA), cyclosporine (NEORAL), sulfasalzine (AZULFIDINE), methotrexate (RHEUMATREX, TREXALL), azathioprine (IMURAN), cyclophosphamide (CYTOXAN), sodium aurothiomalate (Gold) and cyclosporin. In an embodiment, the anti-CD28 agent is administered to the patient in combination with methotrexate. The anti-CD28 agent may be administered sequentially or as simultaneous co-administration and/or as co-formulation with the DMARD (e.g., methotrexate). For example, the anti-CD28 agent may be administered simultaneously with the DMARD (e.g., methotrexate). In another example, the anti-CD28 agent may be administered after administration of the DMARD (e.g., methotrexate). In a further example, the anti-CD28 agent may be administered before administration of the DMARD (e.g., methotrexate). In various embodiments, the patient is undergoing treatment with the DMARD (e.g., methotrexate). In various embodiments, the patient is non-responsive to the DMARD (e.g., methotrexate).

The invention also provides kits that can simplify the administration of any agent described herein. An exemplary kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

In some embodiments, the anti-CD28 agents (including by way of non-limiting example TAB08) described herein, include derivatives that are modified, i.e., by the covalent attachment of any type of molecule to the composition such that covalent attachment does not prevent the activity of the composition. For example, but not by way of limitation, derivatives include composition that have been modified by, inter a/ia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids.

In still other embodiments, the anti-CD28 agents (including by way of non-limiting example TAB08) described herein may be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

In various embodiments, the present invention provides safe and effective doses and regimens for the anti-CD28 agents (including by way of non-limiting example TAB08) described herein. In various embodiments, the anti-CD28 agent is administered as a composition. In various embodiments, the anti-CD28 agent is present in the composition at a concentration of about 0.1 mg/ml to about 15 mg/ml, such as from about 0.5 mg/ml to about 10 mg/ml. In some embodiments, the anti-CD28 agent is present in the composition at a concentration of about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.5 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 10.5 mg/ml, about 11 mg/ml, about 11.5 mg/ml, about 12 mg/ml, about 12.5 mg/ml, about 13 mg/ml, about 13.5 mg/ml, about 14 mg/ml, about 14.5 mg/ml, or about 15 mg/ml, inclusive of all values and ranges therebetween. In various embodiments, the composition may be administered to the patient in a volume of from about 0.1 ml to about 10 ml such as from about 0.1 ml to about 5 ml. For example, the composition may be administered in a volume of about 0.1 ml, about 0.2 ml, about 0.3 ml, about 0.4 ml, about 0.5 ml, about 0.6 ml, about 0.7 ml, about 0.8 ml, about 0.9 ml, about 1 ml, about 1.5 ml, about 2 ml, about 2.5 ml, about 3 ml, about 3.5 ml, about 4 ml, about 4.5 ml, about 5 ml, about 5.5 ml, about 6 ml, about 6.5 ml, about 7 ml, about 7.5 ml, about 8 ml, about 8.5 ml, about 9 ml, about 9.5 ml, or about 10 ml, inclusive of all values and ranges therebetween,

In some embodiments, the anti-CD28 binding agent is administered by intravenous infusion and is infused for a period of at least about 2 hours, or at least about 4 hours, or at least about 8 hours, or at least about 10 hours. In some embodiments, the anti-CD28 binding agent is infused for no more than about 14 hours, about 12 hours, about 10 hours, about 8 hours, about 6 hours, or about 4 hours. In some embodiments, the anti-CD28 binding agent is infused for a period of from about 4 to about 12 hours, or from about 4 to about 10 hours, or from about 4 to about 8 hours, or from about 6 to about 10 hours.

In various embodiments, the patient is monitored during infusion for an increase in one or more of IL-1β, IL-2, IL-4, IL-6, IL-10, IL-17, TNFa, and INFγ in circulation. In various embodiments, the patient is monitored during infusion for an increase in one or more of IL-1β, IL-2, IL-4, IL-6, IL-10, IL-17, TNFa, and INFγ in circulation, to thereby assess activation of Treg cells and/or detect release of pro-inflammatory cytokines. In various embodiments, the infusion of the anti-CD28 agent (including by way of non-limiting example TAB08) is stopped where an substantial increase in proinflammatory cytokines is detected. In various embodiments, the RESTORE system is used to monitorpatients. The RESTORE system is, inter alia, a method for testing a prospective or known immunomodulatory drug for T-cell activation, comprising the step of contacting in-vitro a peripheral blood mononuclear cell (PBMC) culture with a predetermined amount of the prospective or known immunomodulatory drug and observing the PBMC culture for T-cell activation using a readout system, upon contact with the prospective or known immunomodulatory drug, wherein the ceil density of a PBMC preculture is adjusted such that cell-cell contact of the PBMC is enabled and wherein the PBMC preculture is cultured for at least 12 hours and is described in Patent Publication WO/2011/036308, the contents of which are hereby incorporated by reference. In some embodiments, the invention provides a method of treating a patient for an inflammatory disease (e.g., RA) with an anti-CD28 agent (including by way of non-limiting example TAB08) wherein the patient is evaluated with the RESTORE system.

In various embodiments, the treatments of the present invention avoid one or more of the following potential side effects: cytokine storm (e.g. increase in one or more of TNFa alpha, INF gamma, IL-10, IL-2, and IL-6), increased CRP and erythrocyte sedimentation rate, lymphopenia, moncytopenia, thrombocytopenia, disseminated intravascular coagulation, normochromic and/or normocytic anemia, dysplastic neutrophils, capillary leak, hemodynamic instability, lactic academia, early acute renal impairment, urinary sediment, granular casts, acute pulmonary changes, acute lung injury, acute respiratory distress, increased alanine aminotransferase and/or alkaline phosphatase, diffuse erythema, late desquamation, delirium, amnesia, paresthesia and/or local numbness, difficulty concentrating, headache, bowel urgency and/or diarrhea, nausea and/or vomiting, and myalgia (e.g. lower back and calves). Accordingly, in some embodiments, the inventive methods are not characterized by one or more of the described potential side effects. Accordingly, in some embodiments, the present methods provide safe treatment without a toxic cytokine storm.

In various embodiments “cytokine” refers proteins released by one cell population which act on another cell as intercellular mediators, including for example, lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and tumor necrosis factor-β; mullerian-inhibiting substance; mouse gonadotropin-associated peptide; inhibin; activin; vascular endothelial growth factor; integrin; thrombopoietin (TPO); nerve growth factors such as NGFα; platelet-growth factor; transforming growth factors (TGFs) such as TGF-α and TGF-β; insulin-like growth factor-I and -II; erythropoietin (EPO); osteo inductive factors; interferons such as interferon-α, interferon-β and interferon-γ(and interferon type I, II, and III), colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; a tumor necrosis factor such as TNFα or TNFa-β; and other polypeptide factors including LIF and kit ligand (KL). As used herein, the term cytokine includes proteins obtained from natural sources or produced from recombinant bacterial, eukaryotic or mammalian cell culture systems and biologically active equivalents of the native sequence cytokines. In various embodiments, the present methods involve monitoring a patient during infusion anti-CD28 agents (including by way of non-limiting example TAB08) for changes in cytokines and, optionally, terminating infusion if levels deviate substantially from normal.

In various embodiments, the anti-CD28 binding agent (e.g. TAB08) is administered at about 0.1 μg/kg to about 10 μg/kg of patient body weight. In various embodiments, the anti-CD28 binding agent (e.g. TAB08) is administered at about 0.1 μg/kg to about 8 μg/kg of patient body weight, or about 0.1 μg/kg to about 6 μg/kg of patient body weight, or about 0.1 μg/kg to about 4 μg/kg of patient body weight, 0.5 μg/kg to about 10 μg/kg of patient body weight, or about 1 μg/kg to about 10 μg/kg of patient body weight, or about 2 μg/kg to about 10 μg/kg of patient body weight, or about 4 μg/kg to about 10 μg/kg of patient body weight. In various embodiments, the anti-CD28 binding agent (e.g. TAB08) is administered at about 0.5 μg/kg to about 7 μg/kg of patient body weight, or about 1 μg/kg to about 6 μg/kg of patient body weight, or about 2 μg/kg to about 4 μg/kg of patient body weight.

In various embodiments, the anti-CD28 binding agent (e.g. TAB08) is administered about once per week, about once per month, about every other month, or about one to ten times per year, or about 4 to about 12 times per year. In various embodiments, the anti-CD28 binding agent (e.g. TAB08) is administered about once or about twice per week. In some embodiments, the anti-CD28 binding agent (e.g. TAB08) is administered 1 time, or two times, or three times, or four times, or five times, or six times, or seven times, or eight times, or nine times, or ten times per year.

In various embodiments, the anti-CD28 binding agent (e.g. TAB08) is a monoclonal antibody or antigen-binding portion thereof. In one embodiment, the anti-CD28 binding agent is an intact monoclonal antibody. In another embodiment, the anti-CD28 binding agent is an antigen-binding fragment of an antibody (e.g., an antigen-binding fragment of a monoclonal antibody). In various embodiments, the anti-CD28 binding agent may be modified, engineered or chemically conjugated. Examples of antibodies that have been modified or engineered are chimeric antibodies, humanized antibodies, and multispecific antibodies (e.g., bispecific antibodies). Methods for antibody modification or engineering are known in the art. Examples of antigen-binding fragments include Fab, Fab′, F(ab′)2, Fv, single chain antibodies (e.g., scFv), minibodies and diabodies.

In some embodiments, the anti-CD28 binding agent is a superagonist. Exemplary anti-CD28 superagonist antibody is described, for example, in U.S. Pat. No. 8,334,102, which is incorporated by reference herein in its entirety. In an embodiment, the anti-CD28 binding agent preferentially induces the expansion of Treg cells.

In an embodiment, the anti-CD28 binding agent binds to an epitope of CD28 that competes with TAB08. Where the anti-CD28 binding agent competes with TAB08 for binding CD28, the anti-CD28 binding agent inhibits (completely or partially) binding of the TAB08 to a measurable extent. The inhibition of binding may be measured by any of the methods known in the art. In general, the anti-CD28 binding agent is considered to competitively inhibit binding of TAB08 to CD28 if binding of TAB08 to CD28 is reduced by at least about 10%, at least about 20%, at least about 30%, at least about 40%, at least about 50%, at least about 60%, at least about 70%, at least about 80%, or at least about 90%, in the presence of the anti-CD28 binding agent. In some further embodiments, the anti-CD28 binding agent provided herein decreases the binding of TAB08 in a competition assay by about 10%, about 20%, about 30%, about 40%, about 50%, about 60%, about 70%, about 80%, about 90% or about 100%.

In various embodiments, the anti-CD28 binding agent is a monoclonal antibody or antigen-binding portion thereof, and includes one or more CDR sequences as in TAB08. In an embodiment, the anti-CD28 binding agent is TAB08 or includes a light chain or heavy chain sequence of TAB08.

Methods for producing antibodies, such as those disclosed herein, are known in the art. For example, DNA molecules encoding light chain variable regions and/or heavy chain variable regions can be chemically synthesized using the sequence information provided herein. Synthetic DNA molecules can be ligated to other appropriate nucleotide sequences, including, e.g., constant region coding sequences, and expression control sequences, to produce conventional gene expression constructs encoding the desired antibodies. Production of defined gene constructs is within routine skill in the art. Alternatively, the sequences provided herein can be cloned out of hybridomas by conventional hybridization techniques or polymerase chain reaction (PCR) techniques, using synthetic nucleic acid probes whose sequences are based on sequence information provided herein, or prior art sequence information regarding genes encoding the heavy and light chains of murine antibodies in hybridoma cells.

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, IgG enhancers, and various introns. This expression vector optionally contains sequences encoding all or part of a constant region, enabling an entire, or a part of, a heavy or light chain to be expressed. The gene construct can be introduced into eukaryotic host cells using conventional techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In other embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In still other embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region).

A polypeptide comprising an immunoglobulin heavy chain variable region or light chain variable region can be produced by growing a host cell transfected with an expression vector encoding such variable region, under conditions that permit expression of the polypeptide. Following expression, the polypeptide can be harvested and purified using techniques well known in the art, e.g., affinity tags such as glutathione-S-transferase (GST) and histidine tags.

In various embodiments, the anti-CD28 binding agent (e.g. TAB08) is a monoclonal antibody or antigen-binding portion thereof. The monoclonal antibody or an antigen-binding fragment of the antibody can be produced by growing a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment) can be harvested and purified using techniques well known in the art, e.g., Protein A, Protein G, affinity tags such as glutathione-S-transferase (GST) and histidine tags. It is within ordinary skill in the art to express the heavy chain and the light chain from a single expression vector or from two separate expression vectors.

In various embodiments, the anti-CD28 binding agent activates Treg cells and induces an increase in IL-10 production. In some embodiments, the anti-CD28 binding agent induces an increase in the level of IL-10 in the blood plasma of the patient. In some embodiments, the increase in the level of IL-10 in the blood plasma of the patient is observed within a period of about 30 minutes to about 48 hours after administration. For example, the increase in blood plasma IL-10 level may be observed at about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, or about 48 hours after administration, inclusive of all values and ranges therebetween. In various embodiments, the blood plasma level of IL-10 is at a detectable level of greater than about 1 pg/ml, about 2 pg/ml, about 3 pg/ml, about 4 pg/ml, about 5 pg/ml, about 6 pg/ml, about 7 pg/ml, about 8 pg/ml, about 9 pg/ml, or about 10 pg/ml, inclusive of all values and ranges therebetween.

In other embodiments, the anti-CD28 binding agent does not induce substantial release of pro-inflammatory cytokines. Exemplary pro-inflammatory cytokines includes, but are not limited to, IL-2, TNFa, and INFγ. In an embodiment, the anti-CD28 binding agent activates Treg cells and enhances in CTLA-4 expression.

In various embodiments, administration of the anti-CD28 binding agent effectively reduces inflammation, controls cytokine release, and/or modulate the immune response of the patient. For example, the effectiveness of the anti-CD28 binding agent in treating rheumatoid arthritis may be monitored in accordance with the score system set forth by the American College of Rheumatology (ACR). The ACR score represents a percentage. For example, an ACR20 score means that a person's rheumatoid arthritis has improved by 20%, an ACR50 score means it has improved by 50%, and an ACR70 score means it has improved by 70%. In certain embodiments, the anti-CD28 binding agent provides at least about a 20%, 30%, 40%, 50%, 60%, 70%, 80%, or 90% improvement in the ACR score.

In some embodiments, the effectiveness of the anti-CD28 binding agent (for example, an improvement in the ACR score) is observed at about 1 day, about 2 days, about 3 days, about 4 days, about 5 days, about 6 days, about 7 days, about 8 days, about 9 days, about 10 days, about 11 days, about 12 days, about 13 days, about 14 days, or about 15 days after administration of a single dose of the agent. In other embodiments, the effectiveness of the anti-CD28 binding agent (for example, an improvement in the ACR score) is observed at about 1 day, about 2 days, about 3 days, about 4 days, about 5 days, about 6 days, about 7 days, about 8 days, about 9 days, about 10 days, about 11 days, about 12 days, about 13 days, about 14 days, or about 15 days after administration of multiple doses of the agent.

The anti-CD28 agents (including by way of non-limiting example TAB08) described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, cam phorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

Further, any anti-CD28 agents (including by way of non-limiting example TAB08) described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

The present invention includes the described anti-CD28 agents (including by way of non-limiting example TAB08) (and/or additional agents) in various formulations. Any anti-CD28 agents (including by way of non-limiting example TAB08) (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the anti-CD28 agents (including by way of non-limiting example TAB08) described herein can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising anti-CD28 agents (including by way of non-limiting example TAB08) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In one embodiment, any anti-CD28 agents (including by way of non-limiting example TAB08) (and/or additional agents) described herein is formulated in accordance with routine procedures as a composition adapted for a mode of administration described herein.

Routes of administration include, for example: intravenous, intradermal, intramuscular, intraperitoneal, subcutaneous, intranasal, epidural, oral, sublingual, intranasal, intracerebral, intravaginal, transdermal, rectally, by inhalation, or topically, particularly to the ears, nose, eyes, or skin. In some embodiments, the administering is effected orally or by parenteral injection. In one embodiment, the anti-CD28 binding agent is administered by intravenous infusion. The mode of administration can be left to the discretion of the practitioner, and depends in-part upon the site of the medical condition. In most instances, administration results in the release of any agent described herein into the bloodstream.

Any anti-CD28 agents (including by way of non-limiting example TAB08) described herein can be administered orally. Such anti-CD28 agents (including by way of non-limiting example TAB08) described herein can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment.

In one embodiment, any anti-CD28 agents (including by way of non-limiting example TAB08) described herein is formulated in accordance with routine procedures as a composition adapted for oral administration to humans. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any anti-CD28 agents (including by way of non-limiting example TAB08) (and/or additional agents) described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art.

While doses and regimens of the anti-CD28 agents (including by way of non-limiting example TAB08) are described herein, in general, for the other agents described herein, the doses that are useful are known to those in the art. For example, doses may be determined with reference Physicians' Desk Reference, 66th Edition, PDR Network; 2012 Edition (Dec. 27, 2011), the contents of which are incorporated by reference in its entirety. In some embodiment, the present invention allows a patient to receive doses that exceed those determined with reference Physicians' Desk Reference. The dosage of the other agents described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

Any anti-CD28 agent, including TAB08 (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Definitions

The following definitions are used in connection with the invention disclosed herein. Unless defined otherwise, all technical and scientific terms used herein have the same meaning as commonly understood to one of skill in the art to which this invention belongs.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder or reduction in toxicity, regardless of whether improvement is realized.

In certain embodiments, a pharmacologically effective amount that will treat inflammation disorders will modulate the symptoms typically by at least about 10%, at least about 20%, at least about 30%, at least about 40%, or at least about 50%. In exemplary embodiments, such modulations will result in, for example, statistically significant and quantifiable changes in the disease progression marker or indicia of toxicity as described herein.

This invention is further illustrated by the following non-limiting examples.

### EXAMPLES

### Example 1

### In Vitro Evaluation of Cytokines

A. Illustrative Materials and Methods

Peripheral Blood Mononuclear Cell (PBMCs)

Human PBMCs from freshly drawn venous blood from RA patients were isolated by density gradient centrifugation with Ficoll, and washed with ice-cold balanced salt solution (BSS) per 0.2% BSA. Patients donated 50 mL per sample under informed consent.

Cell Culture and Stimulation Assays

Cell culture was performed using the RESTORE protocol as described in Patent Publication WO/2011/036308, the contents of which are hereby incorporated by reference. In brief, PBMCs were cultured in supplemented RPMI 1640 containing 10% AB-positive heat inactivated human serum (Sigma) (AB medium) for 2 days at high cell density (1×10⁷/mL) to allow tissue-like interactions. Cells were then harvested and cultured under standard conditions (1×10⁶/mL) in 96 or 48-well flat-bottom tissue culture plates in a final volume of 0.2 or 0.6 mL in a humidified incubator at 37° C. with 5% CO₂. GMP-grade TAB08 was provided by TheraMAB GmbH. Clinical grade OKT3 (Janssen-Cilag) or anti-CD3 antibody (Miltenyi) was used as stimulus in suppression assays. MP (SanofiAventis) was dissolved in water (133.6 μM) and stored at −20° C.

Cell Proliferation Assays

Cell proliferation was measured from day 2 to 3 as radioactivity incorporated from [H³] thymidine (1 μCi per well, Hartmann Analytic GmbH) into DNA, using a liquid scintillation counter (PerkinElmer). Results are expressed as counts per minute (cpm). Alternatively, intracellular Ki67 staining and CFSE dye dilution were used to measure cell proliferation. To assess proliferation by CFSE dilution, PBMCs were incubated with CFSE (Invitrogen, Grand Island, N.Y., USA) for 5 min at 4° C., washed twice with cold RPMI supplemented with 10% fetal calf serum. Dye dilution was visualized by FACS.

Treg-Cell Suppression Assay

CFSE-labeled CD4+ T cells were used as indicator cells together with monocytes and Treg cells from the same donor (CD4+ T cells: CD14+ cells, 3:2). Treg cells were generated by stimulating HD precultured PBMCs for 5 days with TAB08 and enriching Treg cells (CD4+ CD127lo CD25hi) by FACS sorting. After 24 hours, a sample was removed from supernatants of the cocultures for cytokine measurements. After 3 days, cells were harvested and analyzed by flow cytometry for CFSE dilution in the indicator cell population.

Analysis of Cytokine Concentrations

Cell culture supernatants were analyzed for the presence of cytokines by Cytometric Bead Array (CBA, BD Biosciences), using an LSR II flow cytometer (BD Biosciences) following the manufacturer's instructions. Results were analyzed using FCAP Array software (Soft Flow, Inc.).

Cytokine levels in the plasma samples from healthy volunteers (HVs) were measured by CBA Enhanced Sensitivity (BD Biosciences) using an FACSCalibur flow cytometer (BD Biosciences) following the manufacturer's instructions. Results were analyzed using FCAP Array Software.

Antibodies and Flow Cytometry

Anti-human CD4-PeCy5, CD25-FITC, CD152-PE, Foxp3-Alexa647, Ki67-PE antibodies were from Biolegend; anti-human Foxp3-Alexa488, CD3Alexa 647, CD127-Alexa647, and CD25-PE antibodies were from BD Biosciences. Appropriate isotype controls were purchased from each company.

For phenotypic analysis, cells were stained with the appropriate antibodies for 20 min at 4° C., washed once with FACS buffer (PBS, 0.1% BSA, 0.2% NaN3), and fixed with 2% paraformaldehyde. For intracellular staining of Foxp3, Ki67, and CD152, cells were first surface-stained, permeabilized with Fix/Perm (eBioscience), and stained with the appropriate antibodies diluted in Perm/Wash (eBioscience). FACS analysis was performed using an FACSCalibur flow cytometer. Data were analyzed using FlowJo Version 9.4.11 software (TreeStar). Results are shown as log 10 fluorescence intensities. To calculate absolute Treg-cell numbers, unlabeled microbeads (BD Biosciences) were added to the stained cells and the following formula was used: Absolute Treg-cell numbers=(Beads used×Treg events)/Beads measured.

Healthy Volunteer Study

The study was carried out in accordance with EU GCP guidelines, and in accordance with the International Conference on Harmonized Tripartite Guideline (ICH): GCP Guidelines (ICH E6, 1996), current version (2008) of Declaration of Helsinki, and relevant current legislation of the Russian Federation, from November 2011 to May 2013 at the Municipal Health Care Institution, Clinical Hospital for Emergency Medical Care in Yaroslovl, Russia. In short, cohorts of three male healthy volunteers premedicated with antihistamine and paracetamol were sequentially infused with increasing amounts of TAB08 in a total volume of 500 mL over 4 to 12 hours under 48 hours clinical surveillance followed by repeated clinical tests up to day 57. None of the HVs presented a CRS. Results were evaluated at regular intervals by a safety board before proceeding further. Plasma was isolated via centrifugation and kept at −80° C. until analysis.

Statistical Analyses

To evaluate statistically significant differences, the Friedman test was used, where p<0.05 was considered as significant. Analyses were made using Prism Version 6.0b for Macintosh (GraphPad Software). An unpaired t-test was also used, where p<0.05 was considered as significant. Analysis were made using Prism Version 6.01 for Windows (GraphPad Software).

B. Results

Low CD28SA Concentrations Favor Treg-cell Expansion Over Pro-inflammatory Cytokine Release In Vitro

As previously reported, freshly prepared PBMC cultures failed to respond to soluble TGN1412 (now renamed TAB08). This is in marked contrast to the fulminant in vivo response observed during the FIH TGN1412 trial However, preculture of PBMCs for 2 days at high cell density corrects the functional deficit of circulating T cells by restoring cell contact-dependent tonic TCR signals, which are required for downstream signaling from CD28SA-ligated CD28 molecules. We studied the response to titrated doses of TAB08 in such high-density (HD) precultured PBMC cultures beginning with 1 μg/mL, which results in 60-80% receptor occupancy.

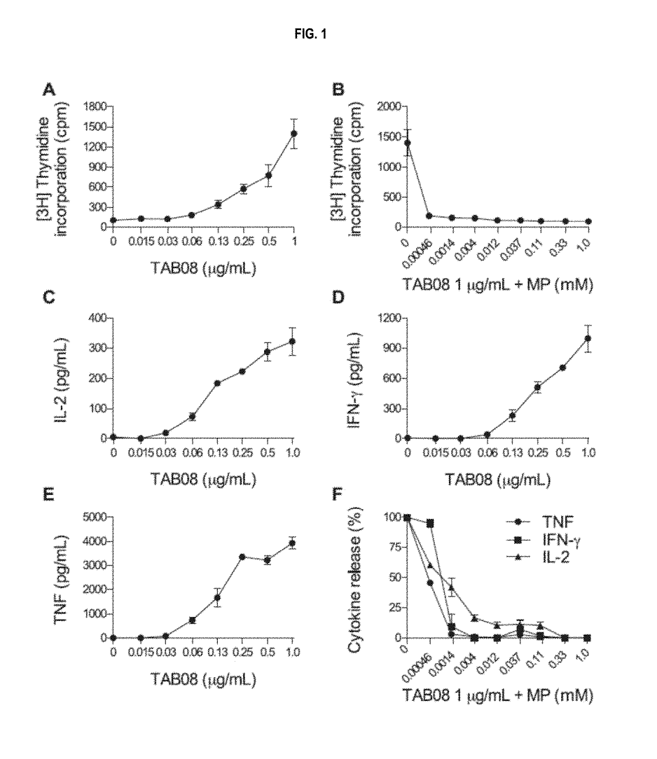

FIG. 1 shows dose—response relationships for PBMCs from a typical healthy donor with regard to proliferation (FIG. 1, panel A) and pro-inflammatory cytokine release (FIG. 1, panels C-E). The lowest TAB08 concentration that triggered detectable cytokine release was 0.06 μg/mL of TAB08, corresponding to only a few percent receptor occupancy. Moreover, methylprednisolone (MP) effectively suppressed both cytokine release and proliferation in a dose-dependent manner (FIGS. 1, panel B and 1, panel F), revealing a particular sensitivity of IFN-γ and TNFa compared with the more resistant IL-2 response.

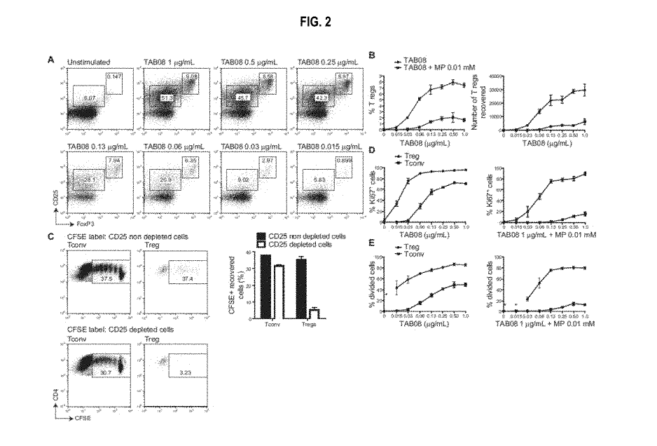

In parallel, cultures were stimulated for 5 days with graded concentrations of TAB08 and evaluated the presence of activated Treg cells. Activated Treg cells were defined as a distinct subset of CD4 T cells expressing high levels of Foxp3 and CD25. As expected, such cells were barely detectable in unstimulated PBMC cultures. As illustrated in FIG. 2, panels A and B, inclusion of TAB08 led to a dose-dependent increase in the relative and absolute number of activated Treg cells, which was apparent with as little as 0.015 μg/mL of the antibody, suggesting their preferential expansion in response to the CD28SA. As expected, CD25 upregulation on Foxp3-negative CD4 T cells was also observed, but it required higher TAB08 concentrations and reached lower CD25 levels than seen on activated Treg cells.

As an alternative to the expansion of preexisting Treg cells, CD28SA-induced conversion of CD4+ Tconv cells toward the Treg-cell phenotype was considered. To test this, HD precultured PBMCs were depleted of CD25+ cells, or left untouched. The cells were then labeled with CFSE for later identification and assessment of cell division, and cocultured with untouched unlabelled PBMCs for TAB08 stimulation. As is shown in FIG. 2, panel C, CD25 depletion did not affect the frequency of recovered CD4+ Tconv cells, which proliferated equally in both settings. In contrast, tenfold fewer Treg cells were recovered from the CD25-depleted as compared with the nondepleted CFSE-labeled input population. Accordingly, the dramatic increase in Treg cells in response to CD28SA stimulation is not due to the conversion of conventional CD4+ Tconv cells but rather to the expansion of preexisting Treg cells.

It was further observed that Treg cells responded to CD28SA stimulation with the expression of the nuclear proliferation marker Ki67 with much greater sensitivity than CD4+ Tconv cells (FIG. 2, panel D). Moreover, using the CFSE dye dilution method to reveal the proliferative history of individual cells, it was found that the activated Treg cells recovered at day 5 had proliferated to a much greater extent than the CD4+ Tconv cells (FIG. 2, panel E). In both assays, this difference was particularly striking at low TAB08 concentrations (0.03 μg/mL or less) where proliferation was restricted to Treg cells. Taken together, these data demonstrate preferential expansion of Treg over Tconv cells under conditions of optimal TAB08 stimulation, which becomes increasingly prominent with reduced CD28SA concentrations, leading to the identification of a threshold below which Treg-cell activation and expansion is observed in the absence of notable Tconv cell proliferation and pro-inflammatory cytokine release.

Corticosteroid-Mediated Suppression of Pro-Inflammatory Cytokine Release Preserves Treq-Cell Expansion

In a parallel set of experiments, the corticosteroid MP was included during the stimulation assays at a concentration (0.01 mM) routinely reached in clinical practice. As shown in FIG. 2, panel B, this resulted in a considerable reduction of Treg-cell activation and numerical expansion. However, this expansion was still obvious under conditions where TNFa and IFN-γ release were fully suppressed (see FIG. 2, panel B versus FIG. 1, panel F). Furthermore, the ability to induce proliferation as assessed by Ki67 expression in Tconv cells was completely abrogated by MP at TAB08 doses still allowing substantial Treg-cell proliferation (FIG. 2, panel D), in agreement with previous animal studies . Finally, the effect of MP inclusion on TAB08-induced cell division was directly compared using CFSE dye dilution. While MP almost fully suppressed division of CD4+ Tconv cells in response to 0.1 μg/mL TAB08 (and approximately by 80% at 1 μg/mL), proliferation of Treg cells proved to be much more corticosteroid-resistant (FIG. 2, panel E).

Suppressive Activity of TAB08-Expanded Treq Cells

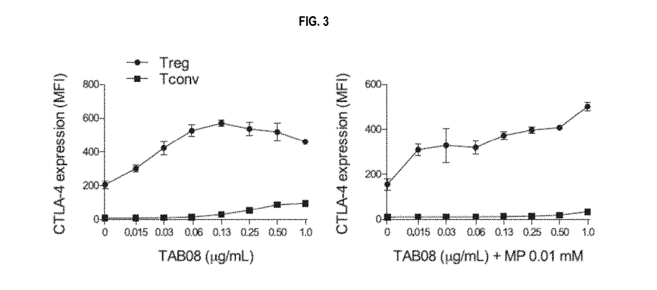

The expression of CTLA-4 (CD152), an important effector of Treg-cell suppression was initially used as a marker for the functional activity of TAB08-expanded Treg cells. In contrast to CD4+ Tconv cells, Treg cells constitutively express detectable levels of CTLA-4. As can be seen in FIG. 3, TAB08 expanded Treg cells strongly upregulated CTLA-4, indicative of increased suppressive activity. It was also observed that the upregulation of CTLA-4 on CD4+ Tconv cells was at a much lower level than found on TAB08-activated or even resting Treg cells. Of note, CD28SA-induced upregulation of CTLA-4 was largely resistant to the inclusion of 0.01 mM MP.

Suppressive activity was then directly measured using a coculture system of purified CFSE-labeled CD4+ T cells and monocytes stimulated with anti-CD3, to which Treg cells, which were prepared by FACS sorting from TAB08-stimulated and HD precultured PBMCs based on high CD25 and low CD127 expression, were added. For

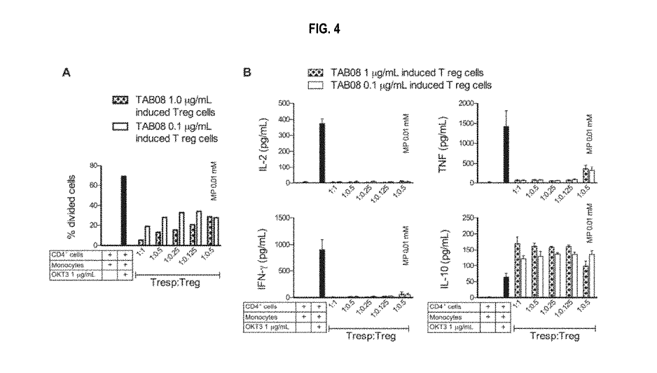

Treg-cell expansion, we used both a high (1 μg/mL) and a low (0.1 μg/mL) concentration of TAB08 with and without the inclusion of 0.01 mM MP. As seen in FIG. 4, panel A, TAB08 expanded Treg cells efficiently suppressed anti-CD3-induced proliferation of CD4+ Tconv cells. This effect was reduced but not extinguished by the inclusion of MP during Treg-cell expansion (FIG. 4, panel A).

The ability of TAB08-expanded Treg cells to suppress anti-CD3-induced cytokine release was also tested. Twenty-four-hour cell culture supernatants of OKT3-stimulated CD4+ T cells and monocytes contained the expected high levels of TNFa and IFN-γ, IL-2, and some IL-10, a key anti-inflammatory mediator that is released by Treg cells. Specifically, inclusion of TAB08-activated Treg cells in this culture system resulted in the absence of IFN-γ, TNFa, and IL-2 in the supernatants, and a marked increase of IL-10 (FIG. 4, panel B). While in the case of IL-2, this effect may partially be due to the ability of Treg cells to act as an IL-2 “sink”, the absence of IFN-γ and TNFa from cultures containing TAB08-expanded Treg cells clearly indicates active suppression, a notion that is further supported by the increased levels of IL-10 in Treg-cell-containing cultures. Suppression of pro-inflammatory cytokine release was observed regardless of whether Treg-cell expansion was driven by 1 or 0.1 μg/mL of TAB08, and was complete with as little as one Treg cell to eight responder cells. Even Treg cells expanded in the presence of MP showed clear suppressive activity. These data indicate that CD28SA stimulation of PBMC results in marked expansion of Treg cells and acquisition of high suppressive activity, and that this effect is observed under conditions of low antibody concentrations or corticosteroid inclusion during Treg-cell expansion, both of which result in the loss of the pro-inflammatory cytokine response.

Cytokine Induction and Treq-Cell Expansion in PBMCs from Rheumatoid Arthritis (RA) Patients

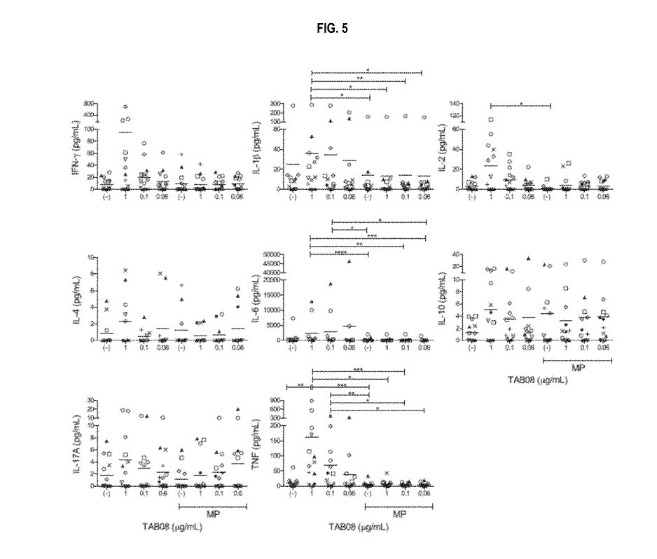

To study whether RA patients might benefit from the observed ability of TAB08 to activate and expand Treg cells, PBMCs from ten RA patients were tested for their response to TAB08 with regard to cytokine release and Treg-cell expansion. No specific exclusion/inclusion criteria were chosen with regard to previous or current medication or disease activity score, resulting in a broad spectrum of disease activity and treatment modalities. As expected, cytokine responses were indeed heterogeneous, but were effectively suppressed by MP where significant induction by TAB08 was seen (FIG. 5). This is particularly clear in the case of TNFa, which plays a key role in RA. Even the high levels of TNFa found in the culture supernatants of PBMC cultures stimulated with 1 μg of TAB08 were reduced to background by MP inclusion.

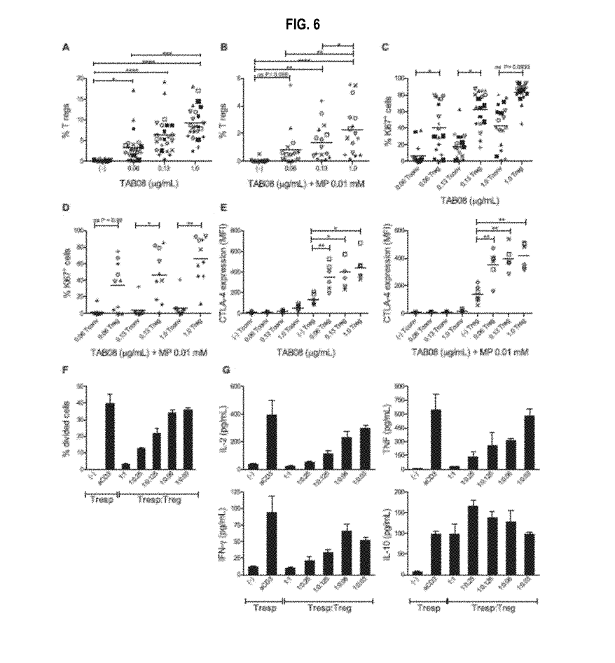

To study Treg-cell activation, the cultures were stimulated for 5 days with a high (1 μg/mL), an intermediate (0.13 μg/mL) and a low (0.06 μg/mL) concentration of TAB08. All samples showed clear-cut Treg-cell responses, which in the vast majority were evident at a concentration of 0.06 μg/mL, the lowest one tested, and reached significance for the average response of all samples over the whole concentration range (FIG. 6, panel A). While the inclusion of MP reduced the increase in activated Treg cells, it was still obvious over the whole dose range of TAB08, and reached statistical significance for the whole group at the intermediate and high concentrations employed (FIG. 6, panel B). As expected, this expansion was associated with the expression of the proliferation marker Ki67, which was found in up to 80% of Treg cells, twice the maximum reached by CD4+ Tconv cells (FIG. 6, panel C). Preferential proliferation of Treg cells was even more evident at lower TAB08 concentrations, consistent with the observations using PBMCs from healthy donors (FIG. 2, panel D), revealing a significantly higher proliferative activity at 0.06 μg/mL. Of note, with the exception of one sample, MP inclusion abrogated Ki67 expression in the CD4+ Tconv cells regardless of the TAB08 concentration employed, but had little effect on the proliferative activity of the activated Treg cell population (FIG. 6, panel D). Taken together, these results indicate the ability of TAB08 to preferentially activate and expand Treg over Tconv CD4+ T cells in PBMCs from RA patients, confirming the initial data obtained with PBMCs from healthy subjects. As observed with those healthy donors, this effect improves toward almost exclusive Treg-cell expansion if low concentrations of the CD28SA are used, or if a corticosteroid is included.

Suppressive Potency of TAB08-Activated Treg Cells from RA Patients

As with healthy donors, the upregulation of the suppressive function of TAB08-expanded Treg cells was tested by measuring the expression of the suppressor—effector molecule CTLA-4. As can be seen in FIG. 3, CTLA-4 upregulation was marked and consistent, even at doses below the induction threshold for pro-inflammatory cytokines in most patients and in the presence of MP, which effectively controlled pro-inflammatory cytokine release (FIG. 5).

We then tested TAB08-expanded Treg cells from a randomly chosen RA patient (RA-7 in Supporting Information Table 1) for their suppressive capacity using the same experimental setup described above for healthy donors (FIG. 4). For Treg-cell expansion, a low dose of 0.06 μg/mL TAB08 was used, which did not induce appreciable pro-inflammatory cytokine release (FIG. 5) but resulted in an increase of activated Treg cells (FIG. 6), which were then purified by cell sorting. As can be seen in FIG. 6, panel F, TAB08-expanded Treg cells effectively suppressed both proliferation and pro-inflammatory cytokine secretion by anti-CD3 activated CD4+ T cells. To test for a possible inhibitory effect of spontaneously produced TNFa on Treg-cell function as has been described for RA patients, the TNFa blocker Enbrel was used during Treg-cell expansion in a parallel experiment. However, this did not lead to further enhancement of suppressive activity (data not shown).

Systemic Release of IL-10, but not Pro-Inflammatory Cytokines, by TAB08 in Healthy Volunteers

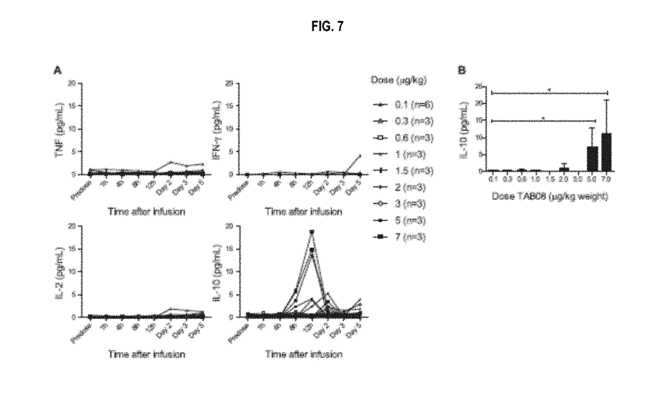

In a FIH trial of TGN1412 in 2006, healthy volunteers (HVs) received a bolus injection of 100 μg/kg body weight, which led to a systemic release of pro-inflammatory cytokines, most notably TNFa, IFN-γ, and IL-2, but also of IL-10, suggesting that both CD4+ EM and Treg cells had been activated. In a new study, much lower doses of TAB08 were applied under close clinical surveillance, starting with 0.1 μg/kg (1000-fold less than applied in the London trial), followed by several intermediate doses and a maximal dose of 7 μg/kg. The antibody was applied by slow infusion (4-12 hours), and the concentrations of IL-1β, IL-2, IL-4, IL-6, IL-10, IL-17, TNFa, and IFN-γ in the volunteers' blood plasma were used for pharmacodynamic assessment of the cytokine response to TAB08. In this study, proinflammatory cytokines remained at baseline level over the full dose-range and observation time as shown in FIG. 7, panel A for the key cytokine release syndrome (CRS)-promoting cytokines TNFa, IFN-γ, and IL-2. This is in contrast with the dramatic elevation of pro-inflammatory cytokines observed in response to the 100 μg/kg TGN1412 bolus injection applied during the 2006 FIH study (in the nanogram per milliliter range) which resulted in a life-threatening CRS. In addition, the anti-inflammatory Treg-cell signature cytokine IL-10 was transiently detected in the plasma of the groups that had received the highest CD28SA doses (FIG. 7, panel A): At 5 and 7 μg/kg, two of three volunteers in each cohort displayed initial IL-10 responses at 8 hours after infusion, which peaked at 12 hours and returned to baseline by day 2 (FIG. 7, panel A). The third member of these cohorts as well as one member of the 1.5 μg/kg cohort showed weaker and delayed IL-10 responses. All other groups remained negative. In spite of the small sample size, the IL-10 response measured at 12 hours reached significance (p<0.05) for the 5 and 7 μg/kg cohorts (FIG. 7, panel B). These findings suggest that tissue-resident Treg cells had responded to TAB08 stimulation by secreting sufficient amounts of IL-10 to be detected in the circulation, and that the threshold level where this becomes effective was around 5 μg/kg, a dose 20-fold below the one applied in the 2006 study.

C. Discussion

Here it was observed that CD28SA TAB08, formerly known as TGN1412, is a potent activator and expander of human Treg cells. Dilution of this stimulatory antibody from the functionally saturating dose applied during the London 2006 Trial results in near-complete loss of the pro-inflammatory cytokine induction, which caused the CRS in that study, while partially preserving Treg-cell activation both in vitro and in vivo. Finally, it was shown that in the presence of the corticosteroid MP at clinically relevant concentrations, which fully suppress toxic cytokine release, Treg-cell activation, and expansion by TAB08 still occurs, thereby providing an additional safety net in particular for patient groups prone to produce high TNFa levels.

With regard to the mechanistic basis for the preferential activation of Treg over Tconv cells by CD28SA stimulation, which was first observed in rodents and has presently been verified in humans, data suggests that this is due to the dependence of the CD28SA-transduced signaling cascade on substrates generated by the TCR: In CD4+ Tconv cells, these derive from weak or “tonic” TCR/MHC interactions generated during T cells search for infected or transformed cells. In contrast, Treg cells are autoreactive and hence receive stronger TCR stimulation by recognizing their cognate HLA II-presented self-antigens , thereby requiring weaker costimulatory CD28 signals for full activation.

In addition, the IL-2-mediated regulatory loop controlling Treg cell activity supports Treg-cell dominance during the CD28SA response. Thus, while IL-2 is a key growth factor for both Treg and Tconv cells, Treg cells are immediately able to bind IL-2 and respond to it through their constitutively expressed high-affinity IL-2R whereas CD25, the alpha chain required for high-affinity binding, first needs to be induced in Tconv cells. In contrast to consumption, IL-2 production is restricted to Tconv cells, where it depends on CD28-mediated costimulation. Indeed, it is most likely the “CD28 responsive element” in the IL-2 promoter, which results in higher production of IL-2 in TAB08 compared with OKT3 (anti-TCR) stimulated T cells, a finding not observed for TNFa and IFN-γ. Without wishing to be bound by theory, it is believed that at appropriately low concentrations of CD28SA, Treg cells become fully activated by synergistic TCR and CD28 signals, and Tconv cells become sufficiently activated to provide enough IL-2 to support this response without mounting a full proliferative or toxic cytokine response themselves. Once activated and numerically expanded, the Treg-cell population will further quench the Tconv cell-effector response by IL-2 withdrawal and by the plethora of suppressive mechanisms available to these cells.

It was previously shown that CD28SA-driven Treg-cell activation in mice is resistant to doses of dexamethasone, which completely suppressed superantigen-induced systemic cytokine release. Here, it is observed that the toxic cytokine response of human PBMCs is sensitive to suppression by the clinically relevant corticosteroid MP at concentrations below those routinely reached in clinical practice. Importantly, this protective effect leaves significant Treg-cell responses intact, and Treg cells generated in the presence of MP are functional when tested on anti-CD3 stimulated PBMCs.

Preferential suppression by corticosteroids of pro-inflammatory cytokine release as compared with Treg-cell activation may well be related to the higher corticosteroid sensitivity of CD28SA-induced TNFa and IFN-γ compared with IL-2 production (FIG. 1, panel D). This effect could be due to the particularly efficient transcriptional activation of the IL2 gene by CD28SA signaling, but may also be the result of differences between IL2 as compared to TNFa and IFNg regarding the sensitivity of cytokine gene expression to the multiple mechanisms of glucocorticoid receptor mediated suppression. In any case, the biological advantage of a glucocorticoid-resistant component of IL-2 production is obvious: given the nonredundant function of IL-2 in Treg-cell homeostasis and activation, corticosteroids as anti-inflammatory hormones would counteract their purpose if they fully interrupted IL-2 provision to Treg cells.

The in vitro results strongly suggest that much smaller doses of TAB08 than those used during the London Trial could be effective stimulators of the Treg-cell compartment in humans, and that corticosteroid comedication could provide an additional safety net for CD28SA therapy of autoimmune and inflammatory conditions.

With regard to low-dose application, it is shown in a new HV trial that TAB08 can indeed be safely applied to humans and stimulate an anti-inflammatory IL-10 response. Besides choosing a 1000-fold lower starting dose than during the FIH study in 2006, the CD28SA was slowly infused, avoiding high local concentrations. While no signs of systemic drug-related inflammatory responses were registered, and pro-inflammatory cytokines remained at base level, a clear-cut IL-10 release was observed in the highest-dosed treatment groups. Since Treg cells (like all T cells) do not respond to TAB08 while in circulation, this response is most likely explained, without wishing to be bound by theory, by the activation of tissue-resident Treg cells. Indeed, it is found that Treg cells present in tonsillar or intestinal lamina propria lymphocytes are activated by lower concentrations of TAB08 to proliferate and to produce IL-10 than is required to activate Tconv cells (unpublished observations).

In a preclinical work, significant therapeutic effects of CD28SA treatment were seen both in the rat adjuvant arthritis model and in mice with systemic TNFa expression resulting in osteolysis. The present results using PBMCs from healthy donors and from RA patients suggest that CD28SA-activated Treg cells may be similarly effective in human autoimmune and inflammatory conditions. Specifically, it is shown that CD28SA activated and CD28SA-expanded human Treg cells are very potent suppressors of inflammatory cytokine release triggered by TCR stimulation, and that the pathway of CD28SA-driven polyclonal Treg-cell activation is functional in RA patients. In this context, it is important to note that Treg cells from patients undergoing active RA are functionally impaired, and that this defect is due to TNFa-driven dephosphorylation of Foxp3. Importantly, Treg-cell function is restored by therapeutic neutralization of TNFa. In the cohort of RA patients, however, robust upregulation of the Foxp3-controlled Treg-cell-effector molecule CTLA-4 is observed, and strong suppressor function irrespective of TNFa neutralization during Treg-cell expansion is detected.

In conclusion, the present results indicate that a correct choice of CD28SA dose and appropriate comedication can effectively treat RA and other autoimmune and inflammatory diseases through its unique Treg-cell-promoting properties.

### Example 2

### Phase I Study of Healthy Patients

A. Illustrative Study Aims

A primary aim of the study was to assess safety and tolerability of single intravenous infusion (IV) of various doses of TAB08 in adult healthy volunteers (HV). Secondary aims: include (1) safety assessment of study drug dosing with various infusion times—12 hours, 8 hours and 4 hours, (2) assessment of pharmacokinetics and pharmacodynamics of TAB08 in healthy volunteers after single intravenous infusion with different doses of the study drug, and (3) characteristics of biomarkers specific for mode of action of the study drug.

B. Study Plan

a. Description of General Study Design and Plan

The study was carried out in accordance with protocol TAB08/HS/R1, version 1.2 from 31 Oct. 2011 (Annex No 1), and with regards to modifications of the Protocol in accordance with Supplement No 2 (Annex No 2) and Supplement No 3 (Annex No 3). The study consisted of four periods: Screening (up to 8 weeks), Drug Dosing (Day 1), Observation Period and Study Completion visit (Day 141 in Cohorts 1-4 and Day 71 in Cohorts 5-9). The study product was examined in 9 dose groups: 0.1 μg/kg, 0.3 μg/kg, 0.6 μg/kg , 1 μg/kg , 1.5 μg/kg, 2 μg/kg, 3 μg/kg, 5 μg/kg and 7 μg/kg.

Healthy volunteers were enrolled to the study in strict sequential order. The infusion in each subsequent dose cohort started after 2-week observation period after completion of infusion for the last HV in the preceding cohort. Each study subject took only one dose of TAB08.

During screening period (for 8 weeks prior Visit on Day 1), a volunteer was given Volunteer Information Sheet for review, he signed consent for participation in the study, laboratory tests were made, and eligibility for the study was assessed. Then each study subject had specific test ex vivo on samples of peripheral blood in RESTORE-system to determine level of individual cytokine release and CD28 receptor occupancy after exposure of blood samples with the product TAB08. The test results were enlisted in inclusion criteria to the study and remained valid for 8 weeks. Standard laboratory blood tests were valid only for 14 days and, if necessary, were repeated for 14 days prior study day 1.

Healthy volunteers which met all inclusion criteria and none of exclusion criteria were enrolled to the study.

The infusion of the first drug dose in Cohort 1 was made in one healthy volunteer (0101). Next 2 subjects (0102 and 0104) were invited for infusion in one week after follow-up of the first volunteer after TAB08 infusion.

Starting dose of the drug product was used to examine safety of drug infusion with various rates—for 12, 8 and 4 hours.

The first subject took complex premedication of intravenous steroids, antihistamine and paracetamol for prevention of reactions related to infusion of the study product. The next HVs which were enrolled to the cohort with the same or higher dose took premedication with antihistamine and paracetamol.

Three to six healthy men were to be enrolled to each cohort:

TABLE 1
Assignment plan per dose cohorts of enrolled subjects.

| | | | Antihistamine (tab.) and | | |
|---|---|---|---|---|---|
| | | Steroids (IV) | paracetamol (tab.) | | Number of |
| Cohort No | Dose (single) | (premedication) | (premedication) | Infusion time hours | volunteers |
| 1 | 0.1 μg/kg | Yes No No No | Yes Yes Yes Yes | 12 12 8 4 | 1 2 1 2 |
| 2 | 0.3 μg/kg | No | Yes | 12 | 3 |
| 3 | 0.6 μg/kg | No | Yes | 12 8 | 1 2 |
| 4 | 1 μg/kg | No | Yes | 8 | 4 |
| 5 | 1.5 μg/kg | No | Yes | 8 | 3 |
| 6 | 2 μg/kg | No | Yes | 8 | 3 |
| 7 | 3 μg/kg | No | Yes | 8 | 3 |
| 8 | 5 μg/kg | No | Yes | 8 | 3 |
| 9 | 7 μg/kg | No | Yes | 8 | 3 |

Study subjects were hospitalized to the study site immediately prior Day 1 (infusion) and were closely monitored within 48 hours after start of drug infusion. After that, they were discharged for out-patient follow-up.

Volunteers returned to the hospital on Day 5, 8, 15, 29, 43 and 57 for clinical tests. Final clinical tests were on Study Completion Visit—for Cohorts 1 to 4—in 3 months (on Week 20 (Day 141)), and for Cohorts 5 to 9—in 2 weeks (on Week 10 (Day 71)).

Starting dose of TAB08 in Cohort No 2 was administered with various rates—the first three volunteers for 12 hours. The fourth subject—for 8 hours, fifth and sixth—for 4 hours.

All three volunteers in Cohort No 2 took infusion for 12 hours. In Cohort No 3—the first subject took 12-hour infusion, next 2 subjects—8-hour infusion. All four subjects in Cohort No 4 took 8-hour infusion of product TAB08. In all subsequent cohorts 5 to 9, infusion time was 8 hours for all enrolled subjects.

In accordance with the protocol, if at least one subject of the cohort experienced dose-limiting toxicity (DLT), then the same dose of the study product should be taken by three additional persons. If any from three additional subjects did not experience DLT (⅙), then dose in the next cohort will be further increased. If one or more additional subjects experienced DLT ( 2/6), then dose escalation would be stopped. Previous dose would be considered as maximum tolerated dose and should be used in phase lb-II clinical study. If two from three subjects enrolled to any of cohorts experienced DLT, then further dose escalation would be stopped, and previous dose would be considered MTD.

Standard safety tests of study drug for study subjects included: physical examination, assessment of vital signs, 12-lead ECG recording, clinical laboratory tests and registration of adverse events.

The decision on subject enrollment to the next cohort with dose escalation and infusion rate was made by Drug Safety Monitoring Board based on clinical and laboratory data after infusion of study drug in the current cohort which were obtained for 2 half-lives of the study drugs after infusion of the study drug which approximately complied with study visit on Week 2.

None of study subjects had signs of dose-limiting toxicity (DLT).

TABLE 2
Study flow chart

| | Screening period | | | | Observation | | | | | | |
|---|---|---|---|---|---|---|---|---|---|---|---|
| | (8 weeks) | | | | period | | | | | | Study |
| | | | Study | Hospitalization | | | | | | Day 57* | completion |
| | | | period | infusion | | | | | | (H8) | Day 141** |
| | Screening | Screening | −14 days | Day 1-3 | | Day8* | Day | Day | Day | Premature | (H20) |
| Procedures | visit 1 | visit 2 | to Day 1 | (Annex 1 B) | Day 5* | (H 1) | (II 2) | (H | (H 6) | discontinuation | Day 71** |
| Demographic data | X | | | | | | | | | | |
| Medical history^{A} | X | | | | | | | | | | |
| ECG | X | | | X | | X | | | | | |
| Vital signs^{B} | X | | X | X | X | X | X | X | X | X | X |
| Physical examination | X | | | X | | X | | | | X | |
| Targeted physical | | | X | X | X | | X | X | X | | X |
| examination | | | | | | | | | | | |
| Premedlcation^{E} | | | | X | | | | | | | |
| TAB08 infusion | | | | X | | | | | | | |

Laboratory

| | | | | | | | | | | | |
|---|---|---|---|---|---|---|---|---|---|---|---|
| Serology tests^{C} | X | | | | | | | | | | |
| Standard laboratory | X | | X | X | X | X | X | X | X | X | X |
| Urinalysis | X | | X | X | | X | X | X | X | X | |
| Urine drug and alcohol | X | | | | | | | | | | |
| Cvtokines | | | | X | X | X | X | | | | |
| Pharmacokinetics and | | | | X | X | X | X | X | X | X | |
| pharmacodynamics | | | | | | | | | | | |
| RESTORE and CD28 | | X*** | | | | | | | | X | |

| | |
|---|---|
| Concomitant therapy | Recorded throughout the study (immediately after |

*Visit is made in the morning.
**Allowable deviation from visit date ±3 Day
***Results of RESTORE and CD28 obtained on screening were considered valid for 8 weeks
^{A}Including Herpes zoster, allergy, tuberculosis history, acute infection, another clinically significant condition
^{B}On all visits, blood pressure, heart rate, breath rate and body temperature will be measured. Body mass and weight will be measured on pre-study visit. Body mass will be also measured on Day 1 (prior infusion), visit on Day 57 and study completion visit.
^{C}HBsAg, HCV, HIV, WR (syphilis), tuberculosis.
^{D}Complete blood count, coagulation test, blood biochemistry. Additional safety laboratory tests may be made only if abnormal test results are obtained in further medical monitoring.
^{E}Only the first subject in Cohort 1 will take premedication by steroids, antihistamine and paracetamol having infusion of the first starting dose. All other subjects will take premedication only with antihistamine and paracetamol.

TABLE 3
Flow chart of procedure in Day 1-3 (hospitalization)

| Day 1 | | | | |
|---|---|---|---|---|
| Prior | Drug infusion | | | |
| infusion | | 30 | Day 2 | Day 3 |

| | | | | | | | | | | | | | | | | | | | |
|---|---|---|---|---|---|---|---|---|---|---|---|---|---|---|---|---|---|---|---|
| Procedures | −1 h | 0 | min | 1 h | 1.5 h | 2 h | 2.5 h | 3 h | 4 h | 5 h | 6 h | 7 h | 8 h | 10 h | 12 h | 14 h | 24 h | 36 h | 48 h |

| | | | | | | | | | | | | | | | | | | | |
|---|---|---|---|---|---|---|---|---|---|---|---|---|---|---|---|---|---|---|---|
| ECG | X | | | | | | | | XG | | | | XG | | XG | | X | | |
| Vital signs^{B} | X | | X | X | X | X | X | X | X | X | X | X | X | X | X | X | X | X | X |
| Comprehensive | X | | | | | | | | | | | | | | | | X | | |
| physical | | | | | | | | | | | | | | | | | | | |
| examination | | | | | | | | | | | | | | | | | | | |
| Targeted physical | | | | | | | | | XG | | | | XG | | XG | | | | X |
| examination | | | | | | | | | | | | | | | | | | | |
| related to subject | | | | | | | | | | | | | | | | | | | |
| complaints | | | | | | | | | | | | | | | | | | | |
| Premedication^{E} | | X | | | | | | | | | | | | | | | | | |

| | |
|---|---|
| TAB08 infusion^{F} | X |

Laboratory procedures

| | | | | | | | |
|---|---|---|---|---|---|---|---|
| Standard | X | | | | | X | |
| laboratory | | | | | | | |
| blood tests^{D} | | | | | | | |
| Urinalysis | X | | | | | X | |
| Cytokines | X | X | X | X | X | X | X |
| Pharmacokinetics | X | | G X | G X | G X | X | X |
| and | | | | | | | |
| pharmacodynamics | | | | | | | |

| | |
|---|---|
| Concomitant | Recorded throughout the study |
| therapy | |

^{B}On all visits, blood pressure, heart rate, breath rate and body temperature will be measured. Body mass and weight will be measured on prestudy visit. Body mass will be also measured on Day 1 (prior infusion), visit on Day 57 and study completion visit.
^{D}Complete blood count, coagulation test, blood biochemistry. Additional safety laboratory tests may be made only if abnormal test results are obtained in further medical monitoring.
^{F}Infusion time depends on the Cohort
G - after infusion for 5 minutes, not using the vein to which infusion catheter is inserted.

b. Screening

Screening procedures were made in 2-3 stages
Screening visit 1—verification of subject compliance with inclusion criteria to the study and absence of exclusion criteria 1-12.
Screening visit 2 was made after investigators obtained results of all tests made on screening visit to take blood samples for tests RESTORE and verification of exclusion criteria No 13 and 14.
Screening visit 3—made if it was necessary to repeat standard laboratory tests. Standard laboratory blood tests were valid only for 14 days.

To determine subject compliance with necessary selection criteria as part of screening visit 1, clinical examination was made which consisted of the following:
selection of demographic data including date of birth, sex, age and race;
medical history including information on chronic infections such as HIV, hepatitis C, hepatitis B, syphilis, herpes, tuberculosis, obtaining allergic history, social habits (urine drug and alcohol test);
vital signs blood pressure, respiration rate, pulse, temperature, height and weight);
general physical (general clinical) examination;
12-lead ECG at rest;
laboratory tests of safety parameters (biochemistry and complete blood count, coagulogram, general urinalysis;
serological testing of blood serum (HBsAg; HCV; HIV; RW; TB);

If a subject met all inclusion criteria and did not meet any exclusion criteria, he was invited to screening visit 2 to:
withdraw blood sample for determination of receptor CD28 occupancy and test in system RESTORE. The results of the test were considered valid for 8 weeks;
electromyography (EMG).

Based on results on RESTORE tests, the decision was made on further participation of subjects in the study. If the first standard laboratory tests were made more than 14 days ago, then such tests (complete blood count and biochemistry test, blood coagulation, urinalysis) were repeated in two-week period prior scheduled dosing day (Day 1).

If a subject met all inclusion criteria and did not have any exclusion criteria, he was enrolled to the study for study therapy.

The investigator completed “Study enrollment approval form” and sent it to the Sponsor representative 1-2 days prior scheduled visit on Day 1. Sponsor representative returned the form to the site specifying cohort and study dose for the subject.

All subjects were hospitalized to the hospital one day prior the study.

c. Diet Standardization, Restrictions

Infusion of the study product was made in fasting conditions—a subject did not take food for 10 hours prior drug dosing. A subject also did not take any liquid 2 hours prior drug dosing except for 100 ml of water to drink with premedication drugs. During infusion, subjects may have meals except for the following products:
grapefruit juice, grapefruit juice drinks,
alcohol,
products containing caffeine or xantines (chocolate, tea, coffee, cola, etc.),
ginger-containing products and beverages.

Since signing Volunteer Information Sheet and Informed Consent Form to study discontinuation, a subject should abstain from alcohol and exclude various excessive physical exercises (sports, horticultural work, visiting baths/saunas, weight lifting, etc.).

A volunteer should not take any drug products (excluding herbal preparations and food supplements).

Subjects should use adequate contraception, along with other contraceptives used by female partners for 3 months after infusion of the study product. Volunteers were informed that they should stay in town for the entire study period.

d. Day 1

Day 1—Prior to Administration of the Study Product

In the morning, on study day 1 prior to infusion of the study product, the following examinations and procedures were made:
physical examination;
vital signs (blood pressure, respiration rate, pulse, temperature, weight);
laboratory tests on safety parameters—biochemistry test and complete blood count, coagulogram, general urinalysis;
12-lead ECG at rest
blood withdrawal for PC and PD tests
blood withdrawal for determination of cytokine levels
premedication.

Blood and urine samples for laboratory tests were taken PRIOR premedication.

To facilitate blood withdrawal and reduce discomfort, the catheter was inserted to one of subject's cubital veins.

Day 1—Infusion of the Study Product

The study product was administered intravenously, by drop infusion with infusion pump. The start time for drug infusion was considered as starting point (“time 0”) to calculate time points of blood withdrawal and procedures for each subject.

Infusion time and dose of product TAB08—depended on the cohort.

Start time, end time of time and interruption period were precisely recorded in the card of each subject.

Day 1—After Administration of the Study Product

Prior infusion of the study product, the following procedures/examinations were made:
pulse, BP, HR and body temperature measurements were made in 0.5, 1, 1.5, 2, 2.5, 3, 4, 5, 6, 7, 8, 10, 12 and 14 hours after start of infusion;
targeted physical examination which was related to complaints of study subjects after infusion;
EGC—after infusion;
blood withdrawal to determine cytokines—in 1, 4, 8 and 12 hours after start of infusion;
blood samples for pharmacokinetic and PD assessments—after infusion (for 5 minutes after infusion, not using the vein to which infusion catheter was inserted);
assessment of adverse events for entire observation period.

e. Day 2
physical examination;
vital signs (blood pressure, respiration rate, pulse, temperature—in 24 hours and additionally in 36 hours after start of infusion;
laboratory tests on safety parameters—biochemistry and complete blood count, coagulogram, general urinalysis;
blood withdrawal for determination of cytokine levels (24 hours after start of infusion);
blood samples for pharmacokinetic and PD assessments would be taken 24 hours after start of infusion;
12-lead ECG at rest;
assessment of adverse events.

f. Day 3
targeted physical examination related to complaints of study subjects;
pulse, BP, HR and body temperature measurements;
blood samples for pharmacokinetic and PD assessments would be taken 48 hours after start of infusion of the study product;
blood withdrawal for determination of cytokine levels—48 hours after start of infusion;
assessment of adverse events.

After abovementioned procedures were made, volunteers left the medical institution.

All subsequent study-related outpatient visits to the hospital were made in the morning which complied with start time of drug infusion on study day related to withdrawal of blood samples for PC and PD assessments.

g. Day 5
Targeted physical examination related to complaints of study subjects;
pulse, BP, HR and body temperature measurements;
laboratory tests on safety parameters—biochemistry test and complete blood count, coagulogram, general urinalysis;
blood samples for pharmacokinetic and PD assessments;
blood withdrawal for determination of cytokine levels;
assessment of adverse events.

h. Day 8 (Week 1)
Physical examination;
vital signs (blood pressure, respiration rate, pulse, temperature);
laboratory tests on safety parameters—biochemistry test and complete blood count, coagulogram, general urinalysis;
blood withdrawal for determination of cytokine levels;
blood samples for pharmacokinetic and PD assessments;
12-lead ECG at rest;
assessment of adverse events.

i. Day 15+1 (Week 2)
Targeted physical examination related to complaints of study subjects;
vital signs (blood pressure, respiration rate, pulse, temperature);
laboratory tests on safety parameters—biochemistry test and complete blood count, coagulogram, general urinalysis;
blood withdrawal for determination of cytokine levels;
blood samples for pharmacokinetic and PD assessments;
assessment of adverse events.

j. Day 29+1 (Week 4)
Targeted physical examination related to complaints of study subjects;
vital signs (blood pressure, respiration rate, pulse, temperature);
laboratory tests on safety parameters—biochemistry test and complete blood count, coagulogram, general urinalysis;
blood samples for pharmacokinetic and PD assessments;
assessment of adverse events.

k. Day 43+1 (Week 6)
targeted physical examination related to complaints of study subjects;
vital signs (blood pressure, respiration rate, pulse, temperature);
laboratory tests on safety parameters—biochemistry test and complete blood count, coagulogram, general urinalysis;
blood samples for pharmacokinetic and PD assessments;
assessment of adverse events.

l. Day 57+1 (Week 8)/Premature Study Withdrawal Visit
physical examination;
vital signs(blood pressure, respiration rate, pulse, temperature, weight);
laboratory tests on safety parameters—biochemistry test and complete blood count, coagulogram, general urinalysis;
blood samples for pharmacokinetic and PD assessments;
laboratory test with RESTORE system;
assessment of adverse events.

m. Day 141+3 (Week 20) for Cohorts 1-4 or Day 71+3 (Week 10)—for Cohorts 5-9—Study Completion

20 weeks after infusion of the study drug (approximately in 5 months), subjects visited the hospital where the following procedures were made:
Targeted physical examination;
vital signs (blood pressure, respiration rate, pulse, temperature, weight);
laboratory tests on safety parameters—blood biochemistry and complete blood count, coagulogram;
electromyography (EMG)
assessment of adverse events.

After that, study in volunteers was considered completed.

n. Premature Study Withdrawal

Subjects were reported that they had right to withdraw from the study in any moment, without any sequellae, not explaining their reasons.

3 premature withdrawals were reported in the study—volunteers 0122 and 0130 discontinued screening period on their own wish, volunteer 0115 was withdrawn from the study due to family reasons after infusion of the study product and completion of visit on Day 3.

o. Data Safety Monitoring Board

To provide independent safety monitoring, conduct of the study and assessment of risk/benefit ratio, the Data Safety Monitoring Board was established. The Board regularly reviewed data of all 9 Cohorts and made recommendations regarding possible continuation of the study.

C. Rationale for Study Design

The proposed study design is widely used and standard for studies of the first phase to assess safety and tolerability, pharmacokinetics and pharmacodynamics of new study products in healthy volunteers. Such design minimizes risks for study subjects (at least 3 subjects in a cohort, close monitoring of subject's condition within 48 hours in the hospital as part of and after infusion, 20-week observation period after drug infusion in the first 4 dose cohorts and 10 weeks—in subsequent cohorts, with special attention to organs/systems which are most sensitive to potential toxic effect of the drug product, decision on dose escalation was made by Data Safety Monitoring Board). On the other hand, the study design allowed to obtain data on safety of the drug product and describe its pharmacokinetics.

D. Selection of Study Population

43 healthy volunteers were screened to the study, and 31 subjects were enrolled to 9 dose groups.

30 persons completed the study in accordance with the protocol. 1 subject discontinued the study on his own wish.

a. Inclusion Criteria
1. Adult males aged 18 to 40 years (including). Male subjects should agree to use adequate contraception, along with other contraceptives used by female partners for 3 months after infusion of the study product. Volunteers were informed that they should stay in town for the entire study period.
2. Body weight of volunteers is at least 60 kg, and body mass (weight in kg/(height in m)2) in the range 20-27.
3. Subject in good physical and mental condition which is defined by medical history, physical examination, vital signs, electrocardiogram and other screening examinations, not having abnormal symptoms (signs), on the investigator' opinion.
4. All subject's laboratory values are within normal range on screening and enrollment stages including baseline cytokine level (TNFα, INFγ, IL-2, IL-6, IL-10), as per RESTORE data.
5. Volunteer has signed, understood and signed informed consent form.

b. Exclusion Criteria
1. History of chronic and relapsing hepatic, renal, lung, allergic, cardiovascular, gastrointestinal, endocrine, hematological or metabolic diseases, central nervous system diseases or immunological, emotional and/or psychiatric disorders.
2. Any abnormal value or results of clinical screening laboratory tests which are considered by the investigator as clinically significant.
3. Clinically significant abnormalities of 12-lead ECG in screening period.
4. History of active tuberculosis (TB) and current TB therapy.
5. Any acute diseases at the study enrollment.
6. Healthy volunteers which have donated blood within 4 weeks prior Day 1.
7. Subjects which are known to have positive hepatitis B, hepatitis C, HIV tests or positive screening hepatitis B (hepatitis B surface antigen B—HBsAg), hepatitis C (anti-HCV antibody C) tests, HIV-antibodies.
8. Volunteers which have taken dose of product TAB08 in the study should not be enrolled again to the study.

Treatment-Related Exclusion Criteria:
9. Any chronic therapy.
10. Subjects which have taken any OTC products (for example, aspirin) within 72 prior dosing of the study product.
11. Volunteers which have taken study therapy within four weeks or five half-lives prior screening to the present study.

Exclusion Criteria Related to Life Style:
12. History of drug abuse, illegal use of drugs or drug abuse within 12 months prior screening visit.

Study-Related Exclusion Criteria:
13. Baseline cytokine level (TNFα, INFγ, IL-2, IL-6, IL-10) which exceeds upper limit of normal, as per RESTORE.
14. The level of proinflammatory cytokines (TNFα and INFγ ) after exposure of cell culture with product TAB08 exceeding upper limit of normal, as per RESTORE.

c. Withdrawal of Volunteers from Treatment or Assessment

Subjects are informed that they have right to withdraw from the study in any moment, without any sanctions, and they are not obliged to give reasons. All study withdrawals should be fully recorded in source documents of study subject.

The investigator may withdraw subjects from the study in any moment if he considers that it is in the best interests of study subject.

A subject may be withdrawn from the study for the following reasons:
Protocol violation including non-compliance of study procedures and refusal to visit the hospital in observation period,
Administrative reasons.

If a subject does not come to the scheduled visit, investigators should try to contact him to ensure that it is not due to adverse events. So if a volunteer decides to withdraw from the study, the investigator should make every attempt to ensure that it is not due to adverse events (i.e. a study subject is not obliged to explain his decisions).

If infusion of the study product is prematurely stopped, the main reason for such discontinuation should be specified in source documents and relevant section of CRF. In accordance with the protocol, such subject may remain in the study for all subsequent visits. In the study, all infusions were made fully.

If adverse events or reactions occurred, investigators made every attempt to make all visits in accordance with the protocol and ensure that subjects completed all visits (Day 141 or Day 71) which allowed obtain the fullest test results.

A premature study withdrawal of patient 0115 (dose cohort No 4 ) was reported. After drug infusion and completion of visit on Day 3, the subject moved to another region due to family reasons and withdrew from the study. Despite numerous attempts of investigators to contact with the volunteer, contact was lost to follow-up.

E. Treatment

a. Therapies

TAB08 for infusion was prepared individually for each subject in accordance with procedures described in Annex 4 to the study protocol.

Infusion time—12 hours, 8 hours or 4 hours (depending on dose cohort and DSMB recommendations), intravenous, made with infusion pump and constant infusion rate.

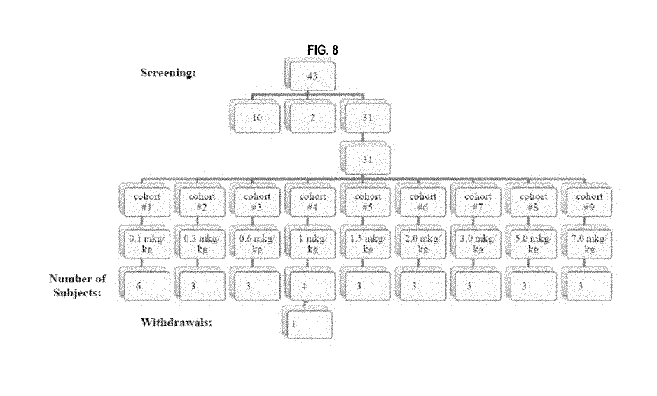

Volunteers were in the study site under constant monitoring for 48 hours from infusion. Test dosages and infusion time in 9 cohorts (see FIG. 8):

Cohort No 1—6 volunteers, dose of the study product—0.1 μg/kg, volunteer 0101 took infusion for 12 hours with premedication of corticosteroids (intravenous), paracetamol and antihistamine;

Volunteers 0102 and 0104 took infusion for 12 hours with premedication of paracetamol and antihistamine;

Volunteer 0105 took infusion for 8 hours with premedication of paracetamol and antihistamine; volunteers 0107 and 0108 took infusion for 4 hours with premedication of paracetamol and antihistamine;

Cohort No 2—3 volunteers, dose of the study product—0.3 μg/kg All volunteers 0103, 0106 and 0112 took infusion for 12 hours with premedication of paracetamol and antihistamine;

Cohort No 3—3 volunteers, dose of the study product—0.6 μg/kg Volunteer 0109 took infusion for 12 hours with premedication of paracetamol and antihistamine; Volunteers 0113 and 0114 took infusion for 8 hours with premedication of paracetamol and antihistamine;

Cohort No 4—4 volunteers, dose of the study product—1.0 μg/kg.

Volunteers 0115, 0116, 0117 and 0118 took infusion for 8 hours with premedication of paracetamol and antihistamine. Subject 0115 was withdrawn from the study after Visit on Day 3 due to family reasons (moving to another reason);

Cohort No 5—3 volunteers, dose of the study product—1.5 μg/kg.

Volunteers 0120, 0123 and 0125 took infusion for 8 hours with premedication of paracetamol and antihistamine;

Cohort No 6—3 volunteers, dose of the study product—2.0 μg/kg.

volunteers 0121, 0126 and 0128 took infusion for 8 hours with premedication of paracetamol and antihistamine;

Cohort No 7—3 volunteers, dose of the study product—3.0 μg/kg.

volunteers 0132, 0139 and 0137 took infusion for 8 hours with premedication of paracetamol and antihistamine;

Cohort No 8—3 volunteers, dose of the study product—5.0 μg/kg.

volunteers 0131, 0133 and 0134 took infusion for 8 hours with premedication of paracetamol and antihistamine;

Cohort No 9—3 volunteers, dose of the study product—7.0 μg/kg.

volunteers 0140, 0141 and 0142 took infusion for 8 hours with premedication of paracetamol and antihistamine.

Actual start and end times of infusion were recorded in case report form of each subject, as well as interruption periods, if applicable.

b. Description of the Study Product(s)

The drug formulation includes water for injection (solvent), acetate buffer 20 mmol/l, solution of sodium chloride 139 mmol/l (isotonic solution) and 0.02% solution of Tween 20 (stabilizer preventing protein aggregation); pH value was adjusted up to 5.5 with acetic acid, and TAB08 concentration (pharmacological active ingredient) is 10 mg/ml. Product TAB08 does not contain excipients of human or animal origin, as well as new chemical compounds. All excipients comply with pharmacopeial standards and are tested in accordance with quality standards of European Pharmacopeia (EP).

Study drug product TAB08 is supplied to the site as concentrate for solution for intravenous infusion which presents clear colorless liquid in glass vials containing 40 ml of TAB08 solution 10 mg/ml. Upon receipt of SP, the study coordinator and/or staff responsible for drug storage checks delivery accuracy and confirms receipt by singing, dating and initialing forwarding documents. A copy of drug delivery form is kept in the Investigator's File at the site.

A drug vial contains label with drug product, batch number, shelf life, concentration, drug storage requirements, clinical protocol number and necessary information.

Unopened drug vials are kept in the site at temperature +2 to +8° C. in dry place protected from light, in a separate safe room with limited access, locked refrigerator with temperature control. There were not deviations from recommended storage conditions.

Solution for infusion is prepared by authorized members of study team having preliminary training. The procedure is fully documented.

The drug accountability is made by study coordinator with drug accountability forms specially designed for the study which are checked by study monitor (“PRUDENTAS” LLC) on each site visit.

The sponsor is responsible for quality of supplied drug product; documents which confirm quality control of the drug product were submitted to the investigator and kept with study documents.

Upon completion of the study, all vials with remaining product TAB08 including vials with saline solution which were used for preparation of infusion solution and infusion bags were returned to the warehouse of “Fisher Clinical Services” LLC for subsequent centralized disposal.

c. Patient Assignment to Treatment Groups

The present study was open-label, and therefore randomization of subjects to various treatment groups was not planned. Healthy volunteers were enrolled to the study in strict sequential order, and after confirmation of all required inclusion criteria and lack of exclusion criteria verified as part of screening. Infusion in next dose cohort was started only after 2-week observation period after infusion to the last healthy volunteer in the previous cohort and DSMB approval for dose escalation.

d. Dosages Selection in the Study

For the calculation of the initial dose of TAB08 drug, TheraMAB company has used new pre-clinical studies that have facilitated to get the necessary additional data on the biological activity, safety, and dose-dependent effect of the monoclonal antibody TAB08. The new study program included: the definition of the receptor CD28 occupation using flow cytometry (FACS) and two test systems which gave the opportunity to evaluate the in vitro response on the administration of TAB08 and use of corticosteroids: RESTORE system and a model of Human Artificial Lymph Nodes (HuALN).

Human Artificial Lymph Node Model—this test system allows conduction of comprehensive immunological evaluation of various agents. Combining different types of cells and simulating in vitro immune response involving T and B lymphocytes, this technology allows predicting the immune response and the degree of immunotoxicity. Human leukocytes from healthy adult donors are the basis for the formation of immunocompetent tissue (artificial lymph node) in a cultured system of three-dimensional matrix. Such a system gives an opportunity to observe the number of immune processes and evaluate immunological parameters, which allows evaluating the immunogenetic potential and risk-benefit ratio for any substance with a high degree of reliability.

RESTORE system—this system was designed to study T cell activating agents using peripheral blood mononuclear cells (PBMC). Have been shown that in vitro addition of various doses of TAB08 to a freshly separated fraction of PBMC from healthy donors did not cause proliferation of T-cells or release of proinflammatory cytokines. TAB08 added to the pre-activated for 2 days culture of PBMC (high-density) causes intense cytokine release, which repeats the pattern of “cytokine storm” described during the London clinical trial.

It is assumed that peripheral blood cells in RESTORE system become similar to the tissue lymphocytes. It is known that over 99% of human T-cells are found in the tissues and not in circulation, thus, in vitro studies with the use of such system enables to a large extent predict the response of each individual patient on the drug administration. Studying dose-dependent response using healthy volunteers' blood allowed establishing a threshold concentration of TAB08—about 0.05 mcg/ml which corresponds to about 5% CD28 receptor occupancy and is 20 times lower than the level calculated as a probable drug concentration in the blood of healthy volunteers in London trial of TAB08.

Using analysis of CD28 receptor occupancy by flow cytometry (FACS), it was shown that previously selected for London clinical trials starting dose equal to 0.1 mg/kg body weight resulted in 60-80% occupancy of CD28 receptor. It was found that this value of occupancy of CD28 receptors caused maximal release of pro-inflammatory cytokines, triggering SAEs in volunteers.

It should be noted that the calculation of the new starting dose for the planned first phase clinical study was fulfilled according to the requirements of the European Medicines Agency (EMA): “Guideline on strategies to identify and mitigate risks for first-in-human clinical trials with investigations medicinal products”, EMEA/CHMP/SWP/28367/07, 2007.

According to this directive, studying drugs with established high risk factors, it is required to use an additional method of dosage calculations—MABEL or Minimal Anticipated Biological Effect Level in addition to NOAEL (No Observed Adverse Effect Level). The potential risk of a drug may be associated with the presence or absence of certain data relating to (1) the mechanism of action, (2) the nature of biological targets and/or (3) compliance with animal models (as in the case of TAB08).

To determine MABEL with an acceptable level of safety all of the above mentioned pre-clinical in vitro studies were used: (1) RESTORE, (2) HuALN and (3) determination of the level of receptor CD28 occupancy. “Concentration/dose-effect” curves were used as the basis of TAB08 safety starting dose calculation which causes minimal expected biological effect in each of the test systems.

Calculating the starting dose of TAB08 using MABEL method an additional 20-fold safety coefficient was applied in order to further limit possible adverse reactions in humans.

Thus, new TAB08 preclinical studies have shown that MABEL level corresponds to 2 mg/kg body weight, which turned out to be 50 times lower the dose level chosen for the first study in England. With additional 20-fold safety coefficient the starting dose was 1000 times lower than administered in 2006 in London to healthy volunteers and was equal to 0.1 mcg/kg with subsequent doses escalation: 0.3 mcg/kg, 0.6 mcg/kg, 1 mcg/kg, 1.5 mcg/kg and 2 mcg/kg. A further ability of the study drug TAB08 dose increase was included to the clinical trial protocol due to the fact that expected doses did not achieve dose-limiting toxicity. In the Amendment #3 to the Protocol it was included a possibility of additional enrollment of volunteers in three dose cohorts: cohort 7—the dose of 3 mcg/kg, cohort 8—the dose of 5 mcg/kg, cohort 9—the dose of 7 mcg/kg.

Healthy volunteers were included in all 9 Cohorts and completed the study according to the study protocol.

e. Infusion Time and Dose Selection for Each Patient

Each study participant received only one dose of TAB08, which depended on the number of cohort to which a volunteer was included. The recommended rate of drug administration and the transition to a subsequent dose cohort was defined by ISMB. The dose calculation depended on the weight of volunteer and was performed directly on the day of infusion. The drug was prepared by authorized personnel under the supervision of the study coordinator and directly before the infusion.

f. Blinding

This study was open.

g. Previous and Concomitant Therapy

To control the release of cytokines triggered by TAB08 and to prevent the development of cytokine release syndrome volunteers received premedication with antihistamines, acetaminophen and corticosteroids. It is well known that corticosteroids are used as a standard prophylactic treatment during immunosuppressive therapy with monoclonal antibody.

To prevent the reactions during infusion, all volunteers received the following premedication:
1. Acetaminophen, 1000 mg, in tablets, 1 hour prior to infusion
2. Antihistamine drug-cetirizine, 10 mg, in tablets—1 hour prior to infusion. The first volunteer from Cohort No 1 (0101) received additionally:
3. IV steroid—methyl prednisolone 100 mg, iv—1 hour prior to infusion.

Investigators were instructed that in case of clinical necessity (the development of adverse reactions) any volunteer can be administered antiemetic drugs, steroids.

h. Compliancy

All TAB08 infusions were carried out in the intensive care unit of the study site. Volunteers remained in the institution for 48 hours after the infusion under the constant supervision of physicians-investigators. Data on the administered dose of TAB08 calculated in accordance with the weight of the volunteer, the exact start time and end time of infusion, interruptions of the infusion was recorded in the source documentation of the volunteer and transferred to the electronic Case Report Forms of the volunteer.

F. Safety and Efficacy Parameters

a. Determination of Efficacy and Safety Parameters and Scheme Efficacy Parameters Measurement

This is a phase I study and its main purpose is evaluation of safety and pharmacokinetics of TAB08. Therefore, efficacy was not evaluated in this study.

b. Safety Parameters Measurement

All of the volunteers, who took the drug TAB08, were included in the safety analysis, according to the principle of planned-to-treat population.
Safety of the volunteers has been constantly assessed throughout the study using the following criteria:
Adverse events and serious adverse events (AEs and SAEs)
Evaluation of changes of routine laboratory tests data (mainly the parameters of complete blood count and biochemistry panel). In addition, the results of laboratory tests are summarized in the table relative to normal laboratory values, these tables, along with graphs of distributions are presented in the statistical report to this study and will be provided upon request.

c. Proper Measurements

Safety parameters were assessed throughout the study. Deviations from the normal values obtained in the course of the study were evaluated by the Investigators as a clinically significant or clinically insignificant.

TABLE 4
Safety laboratory parameters.

```
1. Biochemistry panel -
      Alanine aminotransferase (ALT)
      Aspartate aminotransferase (AST);
      Alkaline phosphatase (ALP);
      Albumin;
      Bilirubin (total);
      Creatinine;
      Urea;
      Total CPK;
      C-reactive protein;
      Glucose;
      Sodium;
      Potassium;
      Total protein;
      Myoglobin;
      Calcium;
      Phosphorus;
      Uric acid
2. Complete blood count
      Hemoglobin;
      Hematocrit;
      Erythrocyte count;
      Platelets count;
      Leukocytes count and leukocyte formula
      (neutrophils, eosinophils, basophils, lymphocytes, monocytes);
      ESR
3. Urinalysis
      Color;
      Transparency;
      Specific gravity;
      Identification of pH;
      Identification of blood with the test strip;
      Identification of leukocytes using a test strip;
      Identification of protein with the test strip;
      Identification of glucose using test strips;
      Identification of ketones using test strips;
      Identification of bilirubin using test strips
Microscopic examination is conducted in case test strip result
shows ++ and more.
4. Coagulation profile
      Prothrombin time;
      INR
```

Registration of Vital Signs

Vital signs included: measurement of blood pressure, heart rate, respiratory rate and body temperature and were measured at each study visit. During infusion (Day 1) these parameters were measured after 0.5, 1, 1.5, 2, 2.5, 3, 4, 5, 6, 7, 8, 10, 12 and 14 hours after the start of infusion.

ECG Evaluation

ECG data evaluation was conducted on screening, before the start of infusion, after the end of infusion, on Day 2 and Day 8.

Main Efficacy Parameters

Since this is a phase I study, the main objective was to evaluate the safety and pharmacokinetics of TAB08. Therefore, efficacy was not evaluated in this study. Efficacy is described elsewhere herein.

Pharmacokinetic Parameters and Pharmacokinetic Evaluation

PK evaluation was conducted on TAB 08 serum concentration from serum collected from each subject received the study drug. The data is presented and described in detail in Section 11.4, where the results are presented in tabular and graphical form and described in accordance with the received dose of the drug. These tables include the number of observations, mean, standard deviation, median, minimum and maximum values and standard pharmacokinetic parameters. Detailed pharmacokinetic assessment with the individual values of TAB 08 concentration in the blood serum of the participants of the clinical study are presented in a separate pharmacokinetic report to this study, which can be provided upon request.

Test tubes with serum samples for pharmacokinetic research were stored at the study site at −800 C. Upon accumulation they were sent using the isothermal container with dry ice to <<TheraMAB>> laboratory, Germany, for studying.

Pharmacodynamic Parameters and Pharmacodynamic Evaluation

PD estimation was conducted on serum cytokines and certain levels of lymphocyte subpopulations.

Test tubes with plasma samples for the determination of cytokine levels were stored at the study site at −800 C.

Pharmacokinetics Assessment

Materials and methods. Quantification of TAB08 in the blood serum of healthy volunteers was performed by enzyme-linked immunosorbent assay (ELISA). Concentrations of TAB08 in samples was assessed, based on binding TAB08 with antigen—recombinant protein CD28, immobilized on the plate wells surface, and further binding of the complex by mouse antibodies against human IgG4, labeled with horseradish peroxidase. This complex was assessed with enzyme as a label for signal detection. Based on concentration-time data following pharmacokinetic parameters were estimated: AUC(0→t), AUC(0→∞), Cmax, Tmax, T., k el, CI, MRT, Vd. These parameters were calculated by nonparametric integral statistical moments method with Phoenix WinNonlin 6.2.1.51, Pharsight Corp., USA software.

For each healthy volunteer blood samples for TAB08 concentration assessment were taken pre-dose=0 h (day 1) and then in 9 time points (altogether—10 times):

Day 1—after infusion end=8,083 h (from other vein then infusion catheter was placed);

Day 2—after 24 hours after infusion start (=24 h);

Day 3—after 48 hours after infusion start (=48 h);

Day 5—after 96 hours after infusion start (=96 h);

Day 8—after 168 hours after infusion start (=168 h);

Day 15—after 336 hours after infusion start (=336 h);

Day 29—after 672 hours after infusion start (=672 h);

Day 43—after 1008 hours after infusion start (=1008 h);

Day 57—after 1344 hours after infusion start (=1344 h).

Pharmacokinetics data analysis:

Following parameters were calculated based on concentration-time data by nonparametric integral statistical moments method (software Phoenix WinNonlin 6.2.1.51, Pharsight Corp., USA):

AUC(0→t)—AUClast (h*ng/mL)—area under pharmacokinetic curve “drug concentration—time”. AUC0→t is calculated from administration time to specific time (t last);)

AUC(0→∞)—AUCINF_obs (h*ng/mL)—area under pharmacokinetic curve “drug concentration—time”.

AUC0→∞ is calculated from administration time to specific time infinity;

Tmax (h)—time to maximum drug concentration in blood (or tissue);

Cmax (ng/mL)—maximum drug concentration in blood (or tissue);

kel—Lambda Z (λz) (1/h)—elimination rate constant—parameter for rate of drug elimination from blood (or tissue);

T1/2—HL_Lambda_z (h)—half-life—period, when half of administered drug dose is eliminated from body (HL_Lambda_z=0.693/Lambda Z);

Cl—Cl_F_obs (mL/h)—clearance—blood (or tissue) volume, which is cleared of drug by a given time;

MRT—MRTlast (h)—men drug residual time—integral characteristic of distribution and elimination processes (MRTlast=AUMClast/AUClast).

Vd—Vz_F_obs—apparent volume of distribution

Vss—Vss_obs—apparent volume of distribution in steady-state.

Data on TAB08 quantification in healthy volunteers' blood serum samples, performed by ELISA for assessment of pharmacokinetics after single intravenous infusion, are presented in the separate pharmacokinetics report available upon request.

Blood samples assessment for all subjects before TAB08 administration (samples at Day 1 before infusion) didn't found testing substance in blood serum samples. For the following time points TAB08 concentrations sufficient for analysis were found not in all samples. For subjects, who received TAB08 at doses 0.1, 0.3 and 0.6 μg/kg, drug was not defined at any sampling time point. For subjects, who received TAB08 at doses 1.0, 1.5 and 2.0 μg/kg drug was defined accidentally, which made impossible relevant pharmacokinetic data analysis with Phoenix WinNonlin 6.2.1.51 software. Hence, for source data of cohorts #1-6 it was impossible to perform pharmacokinetic analysis.

Pharmacokinetic analysis was executable only for subjects from cohorts #7-9, with only one subject (0139) for cohort #7 (dose 3 μg/kg), and only two subjects per cohort for cohorts #8 and 9—(dose 5 μg/kg—0131 and 0133; dose 7 μg/kg—0140 and 0141, respectively)—for all these subjects infusion lasted 8 hours.

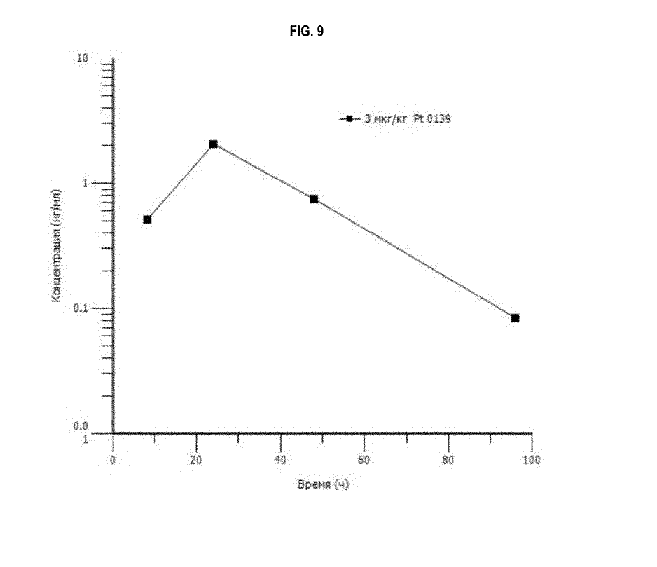

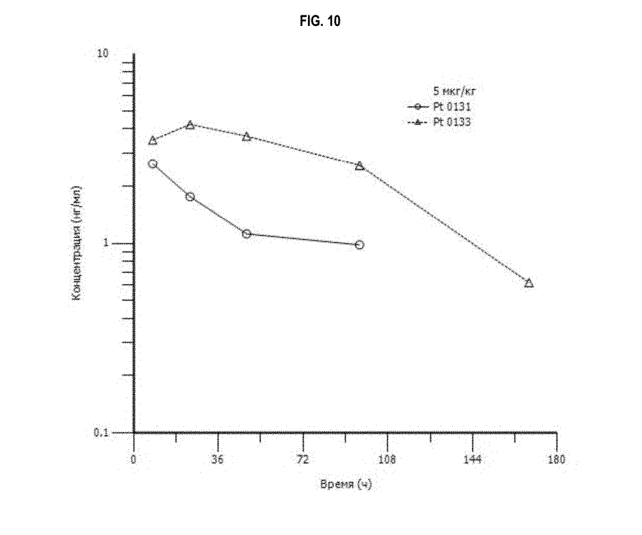

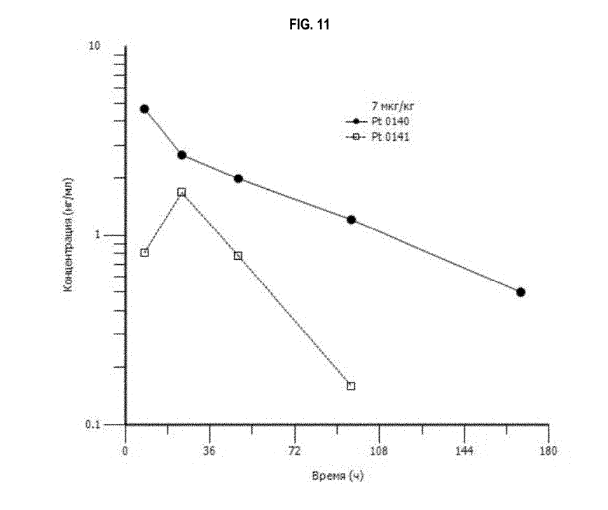

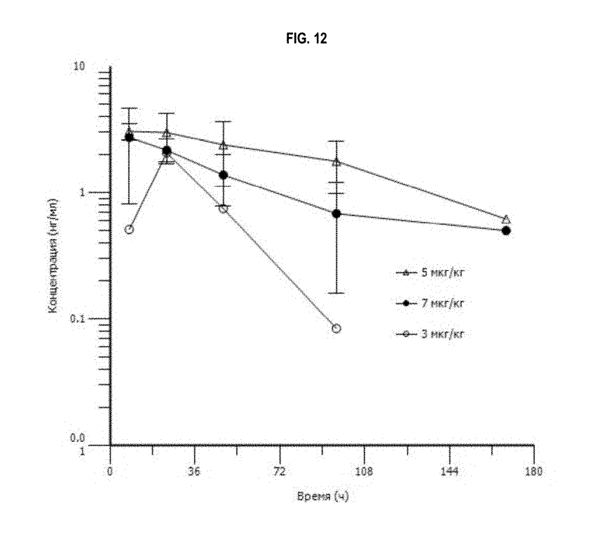

Based on individual concentration-time data averaged parameters were estimated (see Table below). Pharmacokinetic curves are presented on FIGS. 9-12, and main pharmacokinetic parameters are presented in Table 5 below (showing PK assessment descriptive statistics):

TABLE 5

| | | | | | | | | | |
|---|---|---|---|---|---|---|---|---|---|
| Time | Volunteers | Mean | SD | SE | Min | Median | Max | Range | CV |
| (h) | number | (ng/ml) | (ng/ml) | (ng/ml) | (ng/ml) | (ng/ml) | (ng/ml) | (ng/ml) | (%) |

5 μg/kg

| | | | | | | | | | |
|---|---|---|---|---|---|---|---|---|---|
| 0 | 2 | 0 | 0 | 0 | 0 | 0 | 0 | 0 | 0 |
| 8.083 | 2 | 3.06 | 0.62 | 0.44 | 2.62 | 3.06 | 3.50 | 0.88 | 20.34 |
| 24 | 2 | 3.00 | 1.75 | 1.24 | 1.76 | 3.00 | 4.23 | 2.47 | 58.32 |
| 48 | 2 | 2.40 | 1.80 | 1.28 | 1.12 | 2.40 | 3.67 | 2.55 | 75.29 |
| 96 | 2 | 1.78 | 1.12 | 0.80 | 0.98 | 1.78 | 2.57 | 1.59 | 63.34 |
| 168 | 1 | 0.62 | | | 0.62 | 0.62 | 0.62 | | |

7 μg/kg

| | | | | | | | | | |
|---|---|---|---|---|---|---|---|---|---|
| 0 | 2 | 0 | 0 | 0 | 0 | 0 | 0 | 0 | 0 |
| 8.083 | 2 | 2.74 | 2.72 | 1.93 | 0.81 | 2.74 | 4.66 | 3.85 | 99.54 |
| 24 | 2 | 2.17 | 0.69 | 0.49 | 1.68 | 2.17 | 2.66 | 0.98 | 31.93 |
| 48 | 2 | 1.39 | 0.86 | 0.61 | 0.78 | 1.39 | 1.99 | 1.21 | 61.78 |
| 96 | 2 | 0.69 | 0.74 | 0.53 | 0.16 | 0.69 | 1.21 | 1.05 | 108.39 |
| 168 | 1 | 0.50 | | | 0.50 | 0.50 | 0.50 | | |

The following Table 6 shows TAB08 essential pharmacokinetic parameters in blood serum of healthy volunteers after single i.v. infusion for different doses.

TABLE 6

| | | 3 μg/kg | 5 μg/kg | | | | 7 μg/kg | | | |
|---|---|---|---|---|---|---|---|---|---|---|
| | | Subject | Subject | | Mean | | Subject | | Mean | |
| Parameter | Unit | 0139* | 0131 | 0133 | (n = 2) | SD | 0140 | 0141 | (n = 2) | SD |
| AUC_{0→tlast} | ng•h/mL | 76.45 | 130.41 | 435.06 | 282.74 | 215.43 | 271.25 | 75.17 | 173.21 | 138.65 |
| t last | h | 96 | 96 | 168 | — | — | 168 | 96 | — | — |
| AUC_{0→∞} | ng•h/mL | 78.33 | 263.42 | 475.83 | 389.63 | 130.20 | 314.63 | 80.06 | 197.35 | 163.87 |
| Tₘₐₓ | q | 24 | 8.083 | 24 | 16.04 | 11.26 | 8.083 | 24 | 16.04 | 11.26 |
| Cₘₐₓ | ng/ml | 2.07 | 2.62 | 4.23 | 3.43 | 1.14 | 4.66 | 1.68 | 3.17 | 2.11 |
| kₑₗ | 1/h | 0.045 | 0.007 | 0.015 | 0.01 | 0.01 | 0.012 | 0.033 | 0.02 | 0.01 |
| T_{1/2} | h | 15.52 | 94.08 | 45.58 | 69.83 | 34.29 | 60.14 | 21.19 | 40.67 | 27.54 |
| Vd | mL/kg | 857.49 | 2576.23 | 690.95 | 1633.59 | 1333.09 | 1930.39 | 2673.17 | 2301.78 | 525.23 |
| CI | mL/h/kg | 38.30 | 18.89 | 10.51 | 14.74 | 5.99 | 22.25 | 87.43 | 34.84 | 46.09 |
| MRT | h | 29.10 | 36.57 | 58.66 | 47.61 | 15.62 | 51.88 | 30.59 | 41.23 | 15.06 |
| Vss | mL/kg | 1192.95 | 2526.25 | 770.41 | 1648.33 | 1241.56 | 1764.43 | 3165.54 | 2464.99 | 990.74 |

Accordingly, without wishing to be bound by theory, at least the following can be concluded:
TAB08 doses 0.1, 0.3 and 0.6 μg/kg were not detected in any samples at any scheduled PK time point.
TAB08 doses 1.0, 1.5 and 2.0 μg/kg were detected in spot, which made it inappropriate for adequate PK analysis using Phoenix WinNonlin 6.2.1.51 software.
Pharmacokinetic analysis was possible only for HV from cohorts #7-9, with only one volunteer—0139—from cohort #7 (dose 3 μg/kg), and only for two volunteers per cohort for cohorts #8 and 9 (dose 5 μg/kg—0131 and 0133; dose 7 μg/kg—0140 and 0141).
Essential PK parameters were assessed for doses 3, 5 and 7 μg/kg, and no there was no correlation between PK parameters and TAB08 dose. For example, AUC0→t last was 76.45 ng*h/ml in volunteer dosed with 3 μg/kg, while it was 75.17 ng*h/ml in volunteer dosed with 7 μg/kg, and AUC0→t last maximum (435.06 ng*h/m1) was shown in volunteer dosed with 5μg/kg.
Minimal AUC0→∞ values—78.33 and 80.06 ng*h/mL were received when estimating values for subjects with administered doses 3 μg/kg and 7 μg/kg, respectively, maximum value—475.83 ng*h/ml—in subject dosed with 5 μg/kg.
Lowest and highest Cmax values—1.68 ng/ml and 4.66 ng/ml, respectively, were in subjects dosed with 7 μg/kg.
Time to maximum antibody concentration in blood (Tmax) was 8.083—24 hours independently of administered dose (3-7 μg/kg).
Minimum half-life time (T½)—15.52 h was seen in subject dosed with 3 μg/kg, and maximum—94.08 h—in subject dosed with 5 μg/kg.
Minimum kel value (0.007) was in subject, who was dosed by 5 μg/kg, maximum value (0.045)—in subject dosed with 3 μg/kg.
The highest clearance rate (Cl=87.43 mL/h/kg) was in subject, who was dosed by 7 μg/kg, the lowest (Cl=10.51 mL/h/kg)—in subject dosed with 5 μg/kg.
Mean drug residual time (MRT) was lowest in subject, who was dosed by 3 μg/kg-29.10 h, and was within range of 30.59-58.66 in subjects dosed with 5-7 μg/kg.
Minimum Vd value (690.95 mL/kg) was received for s subject, who was dosed by 5 μg/kg, maximum (2576.23 and 2673.17)—in subjects dosed with 5 and 7 μg/kg, respectively.

d. Pharmacodynamic Assessment

Pharmacodynamic assessment of investigational drug TAB08 was performed by evaluating several pro- and anti-inflammatory cytokines in blood serum and by evaluating cell count of T-lymphocytes subsets in peripheral blood samples, which are supposed to mediate TAB08 mechanism of action. Therefore, the capabilities for TAB08 pharmacodynamics assessment in healthy volunteers study are limited due to investigational drug immunomodulating mechanism of action, which is mediated through T-regulatory lymphocytes, and it is supposed that maximum action is realized when immunoregulation mechanisms are affected, which in its course a priori should not be in healthy volunteers.

Cytokines Level in Blood Serum Assessment

During clinical trial in London 2006 it was shown that bolus TAB08 administration in high dose (100 μg/kg dose was used, which is 14.3-fold higher than maximal dose in our clinical study) provoked massive pro-inflammatory cytokines release in 6 out of 6 volunteers, with cytokines concentrations were more that thousand-fold higher than normal levels.

In this trial IL-1β, IL-2, IL-4, IL-6, IL-10, IL-17, TNFα, IFNγ concentrations in volunteers' blood plasma were measured for pharmacodynamic assessment of TAB08 effect on cytokines concentrations. Besides main pro-inflammatory cytokines (IL-1β, IL-2, IL-4, IL-6, IL-17, TNFα, IFNγ), which elevation was detected during clinical trial in London 2006, in this trial was also measured IL-10 concentration, as it's associated with anti-inflammatory effect.

Cytokines concentrations measurement was performed with flow cytometry method with high sensitivity multiplex fluorescent analysis using antibody-coated spheres, specific to each cytokine. This method allows to measure cytokines concentrations in blood plasma with sensitivity range 0.274-200 pg/mL. For this analysis blood samples were taken directly before TAB08 infusion (0 time point), and then after 1, 4, 8 12 hours, and at Days 2, 3, 5, 8 and 15 after infusion. Samples (3 mL) were collected in tubes and then centrifuged (3000 g×10 min). Plasma was stored at −70° C. until analysis, biosamples transportation from study site to the laboratory was done in isothermic shipping containers with dry ice.

Blood plasma samples from 31 volunteers were available for cytokines measurement analysis. For 30 of them there was enough data for statistical analysis. Volunteer's 0115 data were excluded from analysis, because he was withdrawn from study after Day 3 and this subject's further data is handled as lost. For volunteer 0133 from cohort 5 μg/kg the measurement result from day 2 after TAB08 infusion was excluded from analysis, because at that time he had an adverse event—rhinitis, considered as not related to study drug by Principal Investigator. In most cases rhinitis is accompanied by elevated levels of pro-inflammatory cytokines. In this volunteer it was also registered significant transient elevation of IL-6 pro-inflammatory cytokine only, which exceeded higher limit of quantification. There was no elevation of other cytokines concentrations. Additionally in volunteer 0123 from cohort 1.5 μg/kg it was registered transient IL-6 elevation up to 50,24 pg/mL at Day 2 after TAB08 infusion, which is likely to be explained by an adverse event—allergic reaction, considered as related to study drug by Principal Investigator. And there was no elevation of other cytokines concentrations at this time point. This single value was also excluded from statistical analysis.

In all enrolled volunteers levels of IL-1β, IL-2, IL-4, IL-6, IL-10, IL-17, TNFα, and IFNγ during the trial were within physiological normal ranges.

Conclusion and Discussion

In general, during this study cytokines analysis in blood plasma of healthy volunteers did not shown any clinically significant cytokines concentrations elevation after TAB08 infusion, except for two cases of short-term IL-6 elevation, which were definitely timely associated with adverse events with inflammatory pathophysiologic pathway (rhinitis and allergic reaction).

Therewith, there was no statistically significant pro-inflammatory cytokines (IL-1β, IL-2, IL-4, IL-6, IL-17, TNFα, and IFNγ) levels elevation when comparing to baseline concentrations before TAB08 administration, for any TAB08 dose assessed in this trial. Thus it can be assumed that there is no dose-dependence of pro-inflammatory cytokines concentrations from TAB08 dose within assessed TAB08 doses range, which confirms data from ex vivo analyses in RESTORE system.

Additionally in cohorts 8 and 9 (5 and 7 μg/kg, respectively) there was significant antiinflammatory cytokine IL-10 concentrations elevation within time range 8-48 hours after TAB08 infusion, with a maximum at time point “12 hours”, when comparing to 0 time point, which could be considered as a biomarker of pharmacodynamic activity of TAB08. When comparing statistical significance of IL-10 concentrations differences between group with “low” TAB08 doses (cohorts 1-5, doses lower than expected MABEL) and group with “high” doses (cohorts 6-9, expected MABEL dose and higher), it was seen statistically significant IL-10 concentration elevation after 120-360 hours after TAB08 administration at doses 0.1-1.5 μg/kg, while at doses 2-7 μg/kg statistically significant IL-10 concentration elevation was seen after 8-48 hours. This finding could be treated as a trend for dose-dependence of anti-inflammatory cytokine IL-10 concentrations elevation from TAB08 dose, but due to very limited sample size in this study such findings require further assessment.

Lymphocytes Subpopulations Assessment in Peripheral Blood

As a surrogate marker of pharmacodynamic effect in this study was used assessment of T regulatory lymphocytes (T-regs) population, which are deemed to be a key factor in TAB08 mechanism of action. Analysis was performed by flow cytometry method on a flow cytofluorometer COULTER EPICS XL (“Beckman Coulter”, USA) using monoclonal antibodies CD4, CD25, FoxP3 Ki67.

Blood sampling for T-regs measurement was performed immediately before TAB08 administration (pre-dose), immediately after end of infusion (post-dose) and 2, 3, 5, 8, 15, 29, 43, and 57 days after infusion. Blood samples (3 mL) were collected into Leucosep tubes, for optimal isolation of peripheral blood lymphocytes and mononuclear cells (PBMC), and then centrifuged (13000×g during 10 min). Enriched lymphocytes and PBMC fraction was collected, washed and frozen in special freezing medium. Cells were stored at −80° C., samples transportation from investigational site to the laboratory was performed in isothermal transportation containers in dry ice. After thawing, percent of T-regs (FoxP3 marker) and percent of activated T-regs (Ki67 marker among FoxP3 positive cells) was evaluated among all CD4+ cells.

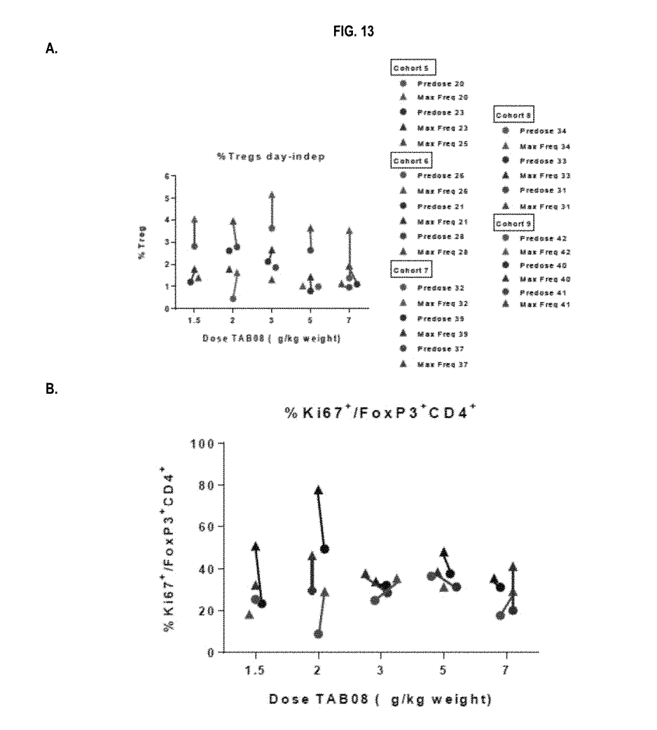

Data for T-regs subsets from cohorts 5-9 is presented. Maximum of T-regs increase and activated T-regs increase in % for cohorts 5-9 (doses 1.5-7 μg/kg, respectively) are shown in FIG. 13 (panels A and B).

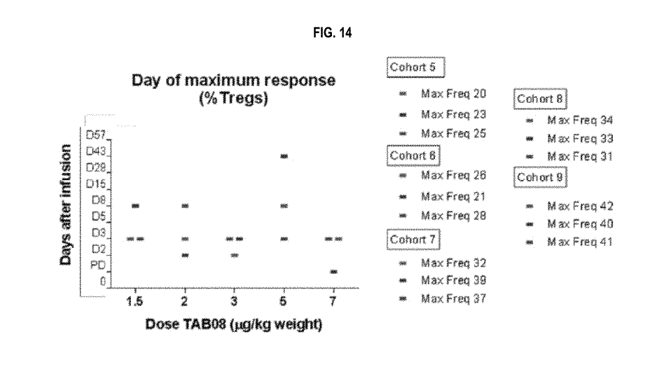

Also time to maximum response in T-regs subset after TAB08 infusion was assessed. It was found that in 8 out of 15 volunteers from cohorts 5-9, maximum of T-regs response was seen on Day 3 after TAB08 infusion, in 2 out of 15 volunteers maximum of T-regs response was seen on Day 2 after infusion, in 3 out of 15 volunteers maximum was seen on Day 8 after infusion, and by one volunteer maximum was seen on <<Post dose>> (immediately after infusion) and on Day 43 after infusion. This data is presented on FIG. 14.

When analyzing T-regs increase when compared to baseline (pre-dose) levels in volunteers at cohorts 5-9, there was no statistically significant dose-dependence of the T-regs response after TAB08 exposure. But taking into account data limitation it's possible that there was a tendency to such dose-dependence, which could be established during further clinical studies.

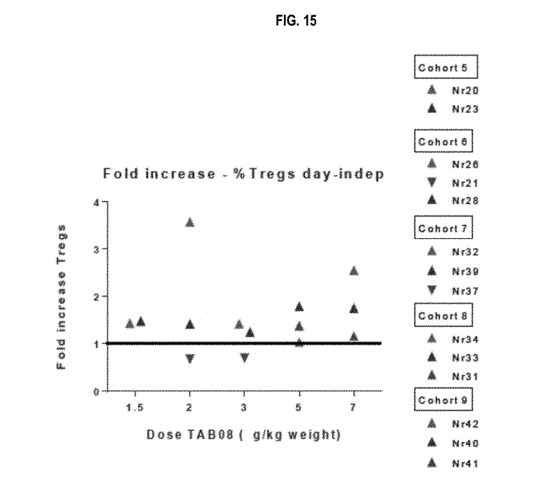

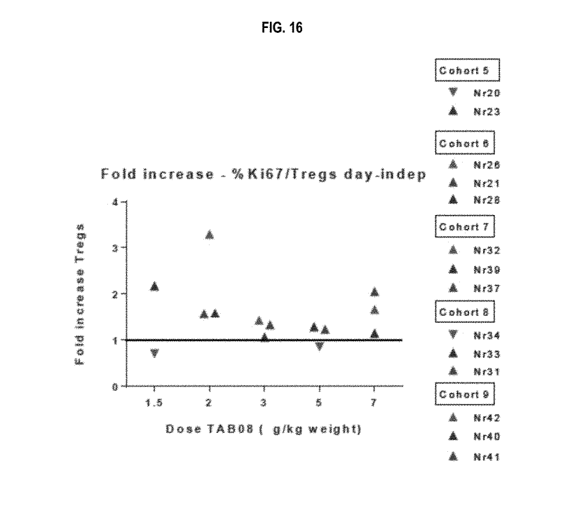

Maximum of T-regs increase and activated T-regs increase in folds compared to pre-dose values is shown on FIGS. 15 and 16.

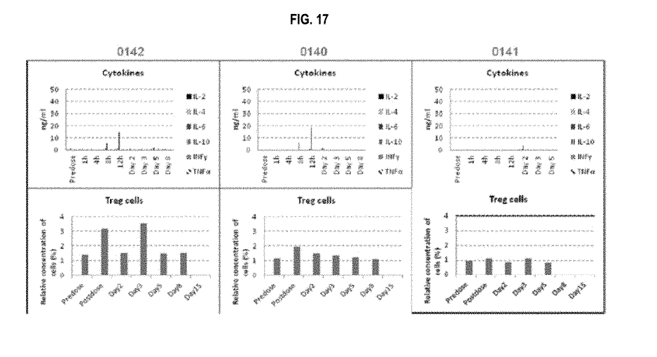

When analyzing T-regs subsets at volunteers from cohort 9 (dose 7 μg/kg) following special feature was noted: increase in T-regs quantity was registered only in those volunteers (0142 and 0140), for which it was also seen increase of anti-inflammatory cytokine IL-10 concentration. For volunteer 0141 there was no significant change in T-regs quantity, as well as no IL-10 elevation in blood. Respective diagrams are presented on FIG. 17.

Conclusion and Discussion

The quantity of T-regs in peripheral blood is relatively low—and consists about 1% from the overall T-regs count in body. Further, of all CD+ cells in blood, T-regs are least presented, when comparing to tissue matrix. Increase of circulating T-regs quantity may be only secondary effect of T-regulatory response in tissues, because, without wishing to be bound by theory, circulating in blood T-regs are unable to respond to TAB08 exposure. It is not clear which TAB08 tissue concentration would induce T-regs proliferation and its appearance in the systemic circulation, which in its turn will allow registering their elevation in systemic circulation. T-regs actively move from blood to tissues and actively react to different stimulus, which impedes faithful interpretation of available data. Nonetheless, available data could directly or proximately indicate on T-regs activation after TAB08 exposure.

Reverse situation was seen with increase of IL-10 concentration: with near-zero background values without TAB08 exposure, even minor IL-10 response can be registered in systemic circulation when using high-sensitivity quantification methods, which was shown in this clinical trial. To be noticed, that during clinical trial in London 2006 there was registered IL-10 levels that were approximately 100-fold higher than the IL-10 response in this clinical trial. But selectiveness of IL-10 increase found in cohorts 8 and 9 (doses 5 and 7 μg/kg, respectively) could evidence about much more selective influence of TAB08 in low doses. Without wishing to be bound by theory, it is believed that IL-10 and T-regs concentrations changes in systemic circulation may correlate with TAB08 clinical efficacy and thus act as biomarkers for TAB08 effect.

e. Ex Vivo Analysis in RESTORE System

The purpose of RESTORE method application was further definition of safety parameters of the use of TAB08 antibodies and the possibilities of participation of volunteers in clinical trials, in particular:

1. Determine the ability of peripheral blood mononuclear cells of healthy volunteers to induce cytokine release in the absence of TAB08 antibody, which allows assessing the hypersensitivity of the cells to TAB08 monoclonal antibody and ensuring that significant inflammatory processes do not occur in the volunteers.

2. Determine the levels of response of peripheral blood mononuclear cells of healthy volunteers to various concentrations of TAB08, i.e. trace the dose-dependence of cytokine release by mononuclear cells exposed to TAB08 ex vivo.

3. Determine the ability of methylprednisolone, which has anti-inflammatory effect, to suppress cytokine release induced by TAB08.

Plasma samples from 37 volunteers were selected for analysis. During the tests in RESTORE system it was revealed that 5 volunteers had elevated (above the physiological medium values) secretion of IL-6 by mononuclear cells prior to their stimulation with TAB08, which however was not accompanied by increased production of other proinflammatory cytokines. All of these volunteers underwent additional testing, which revealed no other abnormalities, and they were allowed to participate in clinical trial. Secretion of IL-6 remained high in the volunteers #0115, 0119 and 0121 on the background of methylprednisolone use, which indicates possible inflammatory processes. All of these volunteers at the end of the convalescence period have passed additional tests according to the standard safety criteria and RESTORE method. Volunteers #0115 and 0121 were revealed no abnormalities and they were included in the clinical trial; #0119 was excluded from the clinical trial.

It should be noted that the maximum doses of TAB08, used for the infusion to volunteers in the first five cohorts were up to 1.5 mcg/kg, which approximately corresponds to a concentration of TAB08 (10 ng/ml) in ex vivo experiments in RESTORE system. In all the examined volunteers the level of IL-2, IL-6, IL-10, TNFα and IFN-γ remained within the physiological norms and the ability of methylprednisolone to inhibit cytokine release in the presence of high doses of TAB08 was maintained. The maximum dose of TAB08 in this study was 7 mcg/kg which approximately corresponds to TAB08 concentration of 50 ng/ml in ex vivo experiments; the cytokine levels also remained within normal limits and the ability of methylprednisolone to inhibit the cytokine release in the presence of high doses of TAB08 was maintained. It should be noted that during the cells stimulation in ex vivo experiments using the TAB08 dose of 1000 ng/ml, which corresponds to the concentration of antibody in the first clinical study TGN1412 in London in 2006 (volunteers were administered a bolus dose of 0.1 mg/kg, i.e. 100 mcg/kg, which is more than 14 times the maximum dose used in this study), peripheral blood mononuclear cells of the most healthy volunteers demonstrated significant release of proinflammatory cytokines such as IL-2, IL-6, TNFα and IFN-γ, that may be an ex vivo predictor of “cytokine storm” syndrome.

The data obtained in the present clinical trial allows us to consider RESTORE system as an adequate means for determining individual sensitivity of mononuclear cells to stimulation with TAB08 drug. This is confirmed by the fact that during this clinical study, none of the volunteers received the infusion of TAB08 had an increased level of cytokines in the blood associated with the effect of the study drug. In view of the obtained data it is assumed that RESTORE method will allow to adequately perform the selection of patients for therapy with TAB08 in the planned clinical studies.

It should also be noted that according to the data obtained in ex vivo analysis of individual sensitivity of mononuclear cells in RESTORE system, a significant release of cytokines by cells was observed at the concentrations of TAB08≧300 ng/ml, which approximately corresponds to the clinical doses ≧30 mcg/kg.

Conclusion

The data obtained in the present study on the tolerability and safety of TAB08, used as a single intravenous infusion at a dose range of 0.1 mcg/kg to 7.0 mcg/kg, suggests a good safety profile and tolerability of TAB08 in the selected range of doses that justifies the conduction of further clinical trials of TAB08 in the populations of patients with the target nosological forms.

H. Conclusions

The results of laboratory tests and clinical examinations of 31 healthy volunteers enrolled in the study to different dose cohorts receiving the TAB08 drug at a dose of 0.1 mcg/kg to 7.0 mcg/kg as a single intravenous infusion administered at a rate of 12, 8 or 4 hours allow for a conclusion of the good tolerability and safety of TAB08 doses in healthy volunteers. The results justify the conduction of further clinical trials of TAB08 in the populations of patients with the target nosological forms.

a. Pharmacokinetic Data

TAB08 at doses of 0.1, 0.3 and 0.6 mcg/kg was not detected in any of the sampling time points for pharmacokinetic studies.

TAB08 in doses of 1.0, 1.5, and 2.0 mcg/kg was detected sporadically, which did not allow the conduction of adequate pharmacokinetic data analysis using Phoenix WinNonlin 6.2.1.51 program.

Pharmacokinetic analysis was possible to conduct only for volunteers from cohorts #7-9, although in cohort #7 (dose of 3 mcg/kg) only for one volunteer—#0139, and in the cohorts #8 and 9—for two (dose of 5 mcg/kg—#0131 and #0133; dose of 7 mcg/kg—#0140 and #0141).

The calculation of basic pharmacokinetic parameters for the doses of 3 mcg/kg, 5 mcg/kg and 7 mcg/kg showed no significant correlations between the dose of TAB08 and pharmacokinetic parameters. So index AUC0→t last had a value of 76.45 ng*h/mL for volunteer administered with a dose of 3 mcg/kg, and 75.17 ng*h/mL for volunteer administered with a dose of 7 mcg/kg, while at the same time the maximum value of this index (435.06 ng*h/mL) was detected in the volunteer administered with a dose of 5 mcg/kg.

b. Pharmacodynamic Data

Discussion on the pharmacodynamic data is based on the obtained during the present study results of studying of the concentration of pro-and anti-inflammatory cytokines, as well as subpopulations of regulatory T-lymphocytes in the blood of the participants of the clinical study.

In general, analyzing the cytokines in the serum of healthy volunteers in the this clinical study there were no clinically significant increases in cytokine concentrations revealed after the infusion of TAB08, except two cases of short-time increase of IL-6, which were clearly associated in time with the development of adverse events with pathophysiological inflammatory component (rhinitis and allergic reaction).

The study showed no dose-dependent effect of TAB08 on the concentration of pro-inflammatory cytokines (IL-1β, IL-2, IL-4, IL-6, IL-17, TNFα and INF-γ) levels in the blood of volunteers in the studied dose range. The absence of such dose-dependence for the used doses range correlates with the data obtained in ex vivo analysis in the RESTORE system, where a statistically significant change in the concentration of pro-inflammatory cytokines was observed at TAB08 concentration of 300 ng/ml, which approximately corresponds to a clinical dose of 30 mcg/kg.

In the cohort #8 and 9 (5 and 7 mcg/kg, respectively) there was noted a significant increase in the concentration of anti-inflammatory cytokine IL-10 in the time range of 8-48 hours after the infusion of TAB08 with the peak at time point “12 hours”, in comparison with the point 0, which can be regarded as a sign of pharmacodynamic activity of TAB08. In this case, there was noted a trend to the correlation between the concentration of IL-10 and the dose of TAB08.

The data from cohorts #5-9 was used (TAB08 doses from 1.5 to 7 mcg/kg, respectively) analyzing the subpopulations of T-regulatory lymphocytes in the blood of participants of the study. It was found that the response of T-regulatory lymphocytes on the impact of TAB08 characterizes by a considerable inter individual variability. Thus, the majority of volunteers included in the analysis had a slightly increased concentration of regulatory T-lymphocytes in the blood, mainly in 1.5-3 times. The maximum increase in the concentration of regulatory T lymphocytes in most of the volunteers was noted on day 3 post TAB08 infusion. No statistically significant dose-dependent response of regulatory T-lymphocytes to the impact of TAB08 was revealed, analyzing the degree of increase of % T-regulatory lymphocytes compared with the baseline levels in volunteers in cohorts #5-9.

Evaluating the changes of regulatory T-lymphocytes concentrations it should be noted that this subpopulation is poorly represented in the blood (about 1% of the total number of lymphocytes), and moreover, actively moves from the blood into tissues and actively responds to various stimuli, which complicates the interpretation of the obtained data. However, this data may directly or indirectly indicate on the activation of regulatory T-lymphocytes exposed to the study drug TAB08, which, however, requires further studying.

In a joint analysis of the concentrations of cytokines and T-regulatory lymphocytes it was revealed that the volunteers in the cohort #9 were observed an associated increase of IL-10 and T-regulatory response being exposed to TAB08, but the interpretation of the identified phenomenon should be considered with caution, since it requires further studying.

In general, obtained in the course of this study data shows that the range of the studied TAB08 doses is safe from the point view of release of pro-inflammatory cytokines. In subsequent clinical trials it is necessary to study whether the identified changes in the concentration of IL-10 and circulating in the blood flow regulatory T-lymphocytes can be correlated with the clinical efficacy of TAB08 and, therefore, act as biological markers during the use of this drug.

### Example 3

### Phase 1b Study With Rheumatoid Arthritis Patients

The primary aim of the study was to assess safety and tolerability of single intravenous infusion (IV) of various doses of TAB08 in adult healthy volunteers (HV).

Secondary aims included safety assessment of study drug dosing with various infusion times—12 h, 8 h and 4 h, assessment of pharmacokinetics and pharmacodynamics of TAB08 in healthy volunteers after single intravenous infusion with different doses of the study drug, and Characteristics of biomarkers specific for mode of action of the study drug.

Objectives: The primary objective was to assess safety and tolerability of several TAB08 doses with multiple i.v. administrations in patients with active Rheumatoid Arthritis (RA) in which standard Methotrexate (MTX) therapy at dose at least 10 mg/week was insufficient.

Secondary objectives included the assessment of TAB08 PK in serum, pharmacodynamics assessment including cytokines, T-cell subsets, CRP, rheumatoid factor, ACCP and anti-DS-DNA levels in blood after each TAB08 administration and efficacy parameters assessment, including ACR 20/50/70 response criteria and its components, DAS20 score, EULAR response criteria.

Methodology: This was an exploratory, multi-center, open-label dose-escalation Phase lb study with multiple i.v. administrations. Standard “3+3” per-cohort study design was used. Patients with active RA despite MTX stable therapy (at least 10 mg/week) were treated by 4 weekly TAB08 i.v. administrations with 8-weeks follow-up period after last administration.

Number of patients (planned and analyzed): It was planned to recruit up to 18 patients in this study. As no dose-limiting toxicity was reached, only 9 patients we enrolled. All of them were included in the analysis of safety and other parameters.

Diagnosis and Main Criteria for Inclusion

The study population consisted of male and female patients aged between 18 and 70 years with a diagnosis of active RA despite MTX stable therapy (at least 10 mg/week). Active RA was defined as minimum 6 out of 66 joints were swollen and minimum 6 out of 68 joints were tender and at least one of the following: CRP level ≧15 mg/L or ESR ≧28 mm/h or morning stiffness >45 min. Seropositive RA defined by rheumatoid factor level ≧14 U/mL or antibodies to cyclic citrullinated peptide (ACCP) level ≧5 U/mL at Screening.

Female patients had to be post-menopausal or surgically sterilized or to use double barrier contraception.

Key exclusion criteria included use of any DMARDs other than MTX within 4 weeks before treatment (8 weeks for leflunomide and biologic antirheumatic agents, 12 month for Rituximab), change in MTX dose within 4 weeks before treatment, any autoimmune disease other than RA, Sjogren's syndrome and Hashimoto's thyroiditis; systemic corticosteroids use at dose>12.5 mg/day of Prednisone equivalent, change of corticosteroid dose or use of intraarticular/intramuscular/intravenous corticosteroid within 4 weeks before treatment, change of NSAID dose within 4 weeks before treatment.

Test product, dose and mode of administration, batch number: All doses were administered via intravenous infusion, with infusion time from 8 to 1 hour.

Duration of treatment: Four single doses on Days 0, 7, 14 and 21.

Reference therapy, dose and mode of administration, batch number: Not applicable in this study.

Criteria for Evaluation

Safety: Primary safety endpoints consisted of adverse events that meet criteria for Dose-Limiting Toxicity (DLT), Maximal Tolerated Dose (MTD) defined as dose level with ≦1 DLT event per 3-6 treated patients, maximal tolerated infusion speed.

Other safety assessments consisted of collecting all adverse events (AEs), serious adverse events (SAEs), with their severity and relationship to study drug, and pregnancies. They included the regular monitoring of hematology, blood chemistry, coagulation profile and urine performed at central laboratory, ECG and regular assessments of vital signs, physical condition and body weight.

Pharmacokinetics: PK parameters after each TAB08 administration included Cₘₐₓ, AUC₍₀₋ₜ₎, AUC_{(0-∞)}, t_{1/2}, λ_{Z}, CL. Steady state parameters included Vₛₛ and C_{trough}.

Pharmacodynamics: PD parameters included
Blood factors levels: rheumatoid factor, antibodies to cyclic citrullinated peptide, C-reactive protein.
T-cell subsets in peripheral blood (PBMC fraction): overall CD4+ T-lymphocytes count, Tregs (CD4+/FoxP3+) count, activated Tregs (CD4+/FoxP3+/Ki67+) count and activated non-Tregs (non-specific T-lymphocytes activation; CD4+/FoxP3−/Ki67+) count.
Blood cytokines levels: INFγ, TNFα, IL-1β, IL-2, IL-4, IL-6, IL-10, IL-17).

Efficacy Efficacy assessments consisted of ACR 20/50/70 response criteria and its components, DAS28 score, EULAR response criteria assessment, Global Disease Assessment scales (Patient and Physician) and Health Assessment Questionnaire (HAQ).

Statistical methods: No statistical hypotheses are testes in this study. Descriptive statistics is used to describe study results.

Summary—Conclusions

Demographic and background characteristics: Nine (9) patients were enrolled into the study. All the patients were Caucasian and female (100%).

Overall study status: All enrolled patients received full course of TAB08 therapy which consisted of four i.v. administrations. Infusions duration varied from 8 hours (1^{st} infusion for each patient) to 1 hour (4^{th} infusions). Investigated TAB08 dose was 5 μg/kg in cohort 1, 7 μg/kg in cohort 2, and 10 μg/kg in cohort 3; 3 patients per each cohort, respectively.

All patients have completed follow-up period (8 weeks after last TAB08 infusion).

Efficacy results: Only ACR criteria response results are available up to date. Efficacy parameters were assessed for all 9 enrolled patients for treatment period. Pre-dose assessment results at Visit 1 (first TAB08 administration) were considered as baseline.

ACR20 response by the End of Treatment (EOT) was seen in 6 out of 9 treated patients, of them 2 patients have reached ACR50 response. These results are summarized in the table below:

TABLE 7

| | Number of patients | | | |
|---|---|---|---|---|
| | with ACR criteria | | | |
| | improvement by EOT | | | Total |
| | ACR 20 | ACR 50 | ACR 70 | patients |
| Cohort 1 | 2 | 1 | 0 | 3 |
| Cohort 2 | 2 | 1 | 0 | 3 |
| Cohort 3 | 2 | 0 | 0 | 3 |
| Total | 6 | 2 | 0 | 9 |
| % of treated | 67% | 22% | 0% | |
| patients | | | | |

It was noted that in each dose cohort 2 out of 3 patients had at least ACR20 response, thus in cohorts 1 and 2 there were 1 patient in each with ACR50 response, with no ACR50 response in cohort 3.

When analyzing core ACR response components by the End of Treatment (EOT), tender joint count (TJC) and swollen joint count (SJC), it was seen that at least 20% improvement for both TJC and SJC was reported in 8 of 9 patients, of them at least 50% improvement was reported in 4 patients, and at least 70% improvement was reported in 1 patient. These results are summarized in the table below:

TABLE 8

| | Number of patients with significant | | | |
|---|---|---|---|---|
| | improvement in number of tender | | | |
| | and swollen joint count by EOT | | | |
| | 20% | 50% | 70% | |
| | improvement | improvement | improvement | Total patients |
| Cohort 1 | 2 | 1 | 1 | 3 |
| Cohort 2 | 3 | 1 | 0 | 3 |
| Cohort 3 | 3 | 2 | 0 | 3 |
| Total | 8 | 4 | 1 | 9 |
| % of treated | 89% | 44% | 11% | |
| patients | | | | |

Sustained ACR20 response by the End of Study (EOS—8 weeks after last TAB08 infusion) was seen in 5 out of 9 treated patients, of them 2 patients have reached ACR50 response and 1 patient have reached ACR70 response. These results are summarized in the table below:

TABLE 9

| | Number of patients | | | |
|---|---|---|---|---|
| | with ACR criteria | | | |
| | improvement by EOS | | | Total |
| | ACR 20 | ACR 50 | ACR 70 | patients |
| Cohort 1 | 2 | 1 | 1 | 3 |
| Cohort 2 | 2 | 1 | 0 | 3 |
| Cohort 3 | 1 | 0 | 0 | 3 |
| Total | 5 | 2 | 1 | 9 |
| % of treated | 56% | 22% | 11% | |
| patients | | | | |

In cohorts 1 and 2 at least ACR20 sustained response was seen in 2 out of 3 patients each, and at least ACR50 sustained response was seen in 1 patient in each cohort. In cohort 1 there was also 1 patient with ACR70 response. However, there was no change since EOT on cohort 3 with only 1 patient with sustained ACR20 response.

When analyzing sustained core ACR response components by the End of Study (EOS)—tender joint count (TJC) and swollen joint count (SJC), it was seen that at least 50% improvement for both TJC and SJC was reported in 5 of 9 patients, furthermore two of them reported at least 70% improvement in both components. These results are summarized in the table below:

TABLE 10

| | Number of patients with significant | | | |
|---|---|---|---|---|
| | improvement in number of | | | |
| | tender and swollen joint count by EOS | | | |
| | 20% | 50% | 70% | |
| | improvement | improvement | improvement | Total patients |
| Cohort 1 | 2 | 2 | 1 | 3 |
| Cohort 2 | 2 | 2 | 1 | 3 |
| Cohort 3 | 1 | 1 | 0 | 3 |
| Total | 5 | 5 | 2 | 9 |
| % of treated | 56% | 56% | 22% | |
| patients | | | | |

In general, TAB08 treatment had beneficial effect in most of enrolled patients. More than half of treated patients had sustained ACR20 response 8 weeks after last TAB08 administration. Full analysis of all efficacy parameters in study population will be performed after all patients complete the study and all data will be gathered.

Conclusion: TAB08 has been shown to be well tolerated and safe in this patient population. Moreover, beneficial treatment effect was reported for most of enrolled patients despite efficacy assessment was not the primary objective of this study. No SAEs, DLTs or other specific unexpected safety signals suspected to be related to study treatment occurred during this study.

These results support favorable risk-benefit ratio for TAB08 use in RA population and justify further clinical studies within TAB08 clinical development program.

Pharmacodynamics Assessment

Pharmacodynamic assessment of investigational drug TAB08 was performed by evaluating several pro- and anti-inflammatory cytokines in blood serum and by evaluating cell count of T-lymphocytes subsets in peripheral blood samples, which are supposed to mediate the TAB08 mechanism of action.

### Effects of TAB08 (Theralizumab) on Regulatory T Cells

### LIST OF ABBREVIATIONS AND DEFINITION OF TERMS

CD Cluster of Differentiation

ICH International Conference on Harmonisation

IFN Interferon

MABEL Minimal Anticipated Biological Effect Level

PBMC Peripheral blood mononuclear cells

PD Pharmacodynamic

TCR T-cell receptor

TNFa Tumor necrosis factor

Treg Regulatory T-Iymphocytes

HV Healthy volunteer

IFN Interferon

IL Interleukin

ELISA Enzyme-linked immunosorbent assay

N/A Not applicable

RA Rheumatoid arthritis

Lymphocytes Subpopulations Assessment in Peripheral Blood

As a surrogate marker of pharmacodynamic effect in this study the assessment of T-regulatory lymphocytes (T-regs) population was used, which are deemed to be a key factor in the TAB08 mechanism of action. Analysis were performed by flow cytometry with a FACSCalibur (BD, USA) using monoclonal antibodies against human CD4, FoxP3 (BioLegend, USA) CD25 and Ki67 (BD, USA).

Blood sampling for T-reg measurement was performed immediately before TAB08 administration (predose), immediately after infusion (postdose), at 12, 24, 48 and 72 hours after each four infusions. Blood samples (3 mL) were collected into Leucosep tubes, for optimal isolation of peripheral blood mononuclear cells (PBMC), and then centrifuged (3000×g during 10 min). Enriched lymphocytes and PBMC fraction was collected, washed and frozen in special freezing medium. Cells were stored at −80° C. Sample transportation from investigational site to the laboratory was performed in isothermal transportation containers in dry ice. After thawing, percent of T-regs (FoxP3 marker) and percent of activated T-regs (Ki67 marker among FoxP3 positive cells) was evaluated among all CD4+ T cells.

Measurement of Regulatory T Cells

TABLE 11
Relative frequencies (%) of regulatory T cells and proliferating
Ki67+-Tregs in the blood of RA patients from first cohort
(5 mkg/kg)

| | |
|---|---|
| % FoxP3+/ | % Ki67+/ |
| CD4+ | FoxP3 + CD4+ |

Patient RA0102 GMP

| | | |
|---|---|---|
| 0102 01 V1-Predose.001 | 1.79 | 8.82 |
| 0102 02 V1-Postdose.002 | 1.89 | 3.51 |
| 0102 03 V1-12h.003 | 2.78 | 12.4 |
| 0102 04 V1-24h.004 | 2.48 | 6.21 |
| 0102 05 V1-48h.005 | 2.58 | 3.25 |
| 0102 06 V1-72h.006 | 2.39 | 4.38 |
| 0102 07 V2-Predose.007 | 1.89 | 14.7 |
| 0102 08 V2-Postdose.008 | 3.36 | 7.95 |
| 0102 09 V2-12h.009 | 1.55 | 9.57 |
| 0102 10 V2-24h.010 | 2.55 | 6.45 |
| 0102 11 V2-48h.011 | 3.09 | 10.7 |
| 0102 12 V3-Predose.012 | 3.24 | 5.85 |
| 0102 13 V3-Postdose.013 | 2.83 | 9.66 |
| 0102 14 V3-12h.014 | 1.84 | 14.3 |
| 0102 15 V3-24h.015 | 2.35 | 18 |
| 0102 16 V3-48h.016 | 3.14 | 19.3 |
| 0102 17 V4-Predose.017 | 2.05 | 19.5 |
| 0102 18 V4-Postdose.018 | 1.89 | 13.8 |
| 0102 19 V4-12h.019 | 2.02 | 21.9 |
| 0102 20 V4-24h.020 | 1.49 | 33.3 |
| 0102 21 V4-48h.021 | 1.82 | 29 |

Patient 0202 AMS

| | | |
|---|---|---|
| RA0202 01 V1-Predose.001 | 1.2 | 24.1 |
| RA0202 02 V1-Postdose.002 | 0.813 | 31.6 |
| RA0202 03 V1-12h.003 | 1.11 | 21.4 |
| RA0202 04 V1-24h.004 | 1.37 | 21.9 |
| RA0202 05 V1-48h.005 | 0.484 | 27.8 |
| RA0202 06 V1-72h.006 | 1.06 | 21.7 |
| RA0202 07 V2-Predose.007 | 0.949 | 16.7 |
| RA0202 08 V2-Postdose.008 | 1.42 | 12.8 |
| RA0202 09 V2-12h.009 | 1.49 | 12.5 |
| RA0202 10 V2-24h.010 | 0.913 | 13.5 |
| RA0202 11 V2-48h.011 | 0.651 | 14.8 |
| RA0202 12 V3-Predose.012 | 1.39 | 26.1 |
| RA0202 13 V3-Postdose.013 | 1.6 | 21 |
| RA0202 14 V3-12h.014 | 1.26 | 28.2 |
| RA0202 15 V3-24h.015 | 0.966 | 23.8 |
| RA0202 16 V3-48h.016 | 0.364 | 28.1 |
| RA0202 17 V4-Predose.017 | 0.864 | 20.9 |
| RA0202 18 V4-Postdose.018 | 1.2 | 22.1 |
| RA0202 19 V4-12h.019 | 0.879 | 35.1 |
| RA0202 20 V4-24h.020 | 0.684 | 16.9 |
| RA0202 21 V4-48h.021 | 0.669 | 26.4 |

Patient 0203 MPS

| | | |
|---|---|---|
| 0103 01 V1-Predose.001 | 0.52 | 29.5 |
| 0103 02 V1-Postdose.002 | 0.685 | 22.9 |
| 0103 03 V1-12h.003 | 0.289 | 25.9 |
| 0103 04 V1-24h.004 | 0.748 | 26.5 |
| 0103 05 V1-48h.005 | 0.965 | 16.1 |
| 0103 06 V1-72h.006 | 0.959 | 24.5 |
| 0103 07 V2-Predose.007 | 1.41 | 26.6 |
| 0103 08 V2-Postdose.008 | 0.727 | 22.1 |
| 0103 09 V2-12h.009 | 0.779 | 24.7 |
| 0103 10 V2-24h.010 | 1.35 | 20.1 |
| 0103 11 V2-48h.011 | 0.855 | 24.9 |
| 0103 12 V3-Predose.012 | 1.08 | 39.2 |
| 0103 13 V3-Postdose.013 | 1.03 | 26.2 |
| 0103 14 V3-12h.014 | 0.975 | 35.8 |
| 0103 15 V3-24h.015 | 1.08 | 17.4 |
| 0103 16 V3-48h.016 | 0.735 | 30.5 |
| 0103 17 V4-Predose.017 | 0.7 | 25.4 |
| 0103 18 V4-Postdose.018 | 0.832 | 23.6 |
| 0103 19 V4-12h.019 | 0.72 | 13 |
| 0103 20 V4-24h.020 | 1.49 | 23.5 |
| 0103 21 V4-48h.021 | 1.08 | 12.7 |

TABLE 12
Relative frequencies (%) of regulatory T cells and proliferating
Ki67+-Tregs in the blood of RA patients from second cohort
(7 mkg/kg)

| | |
|---|---|
| % FoxP3+/CD4+ | % Ki67+/FoxP3 + CD4+ |

Patient 0101 VIU

| | | |
|---|---|---|
| 0101 01 V1-Predose.001 | 0.316 | 8.16 |
| 0101 02 V1-Postdose.002 | 0.363 | 3.77 |
| 0101 03 V1-12h.003 | N/A | N/A |
| 0101 04 V1-24h.004 | N/A | N/A |
| 0101 05 V1-48h.005 | 0.235 | 7.69 |
| 0101 06 V1-72h.006 | 0.326 | 22.2 |
| 0101 07 V2-Predose.007 | 0.449 | 23.7 |
| 0101 08 V2-Postdose.008 | 0.678 | 47 |
| 0101 09 V2-12h.009 | 0.156 | 37.5 |
| 0101 10 V2-24h.010 | 0.255 | 78.9 |
| 0101 11 V2-48h.011 | 0.251 | 45.8 |
| 0101 12 V3-Predose.012 | 0.12 | 7.14 |
| 0101 13 V3-Postdose.013 | 0.348 | 19 |
| 0101 14 V3-12h.014 | 0.073 | 33.3 |
| 0101 15 V3-24h.015 | 0.182 | 3.33 |
| 0101 16 V3-48h.016 | 0.345 | 19.5 |
| 0101 17 V4-Predose.017 | 0.394 | 35.4 |
| 0101 18 V4-Postdose.018 | 0.557 | 43.9 |
| 0101 19 V4-12h.019 | 0.306 | 41 |
| 0101 20 V4-24h.020 | 0.463 | 29.4 |
| 0101 21 V4-48h.021 | 0.263 | 44.7 |

Patient 0204 TFY

| | | |
|---|---|---|
| 0204 01 V1-Predose.001 | 0.58 | 37.9 |
| 0204 02 V1-Postdose.002 | 0.249 | 37 |
| 0204 03 V1-12h.003 | 0.133 | 70.6 |
| 0204 04 V1-24h.004 | 0.266 | 32.4 |
| 0204 05 V1-48h.005 | 0.222 | 21.9 |
| 0204 06 V1-72h.006 | 0.214 | 30.4 |
| 0204 07 V2-Predose.007 | 0.149 | 68.4 |
| 0204 08 V2-Postdose.008 | 0.16 | 40.7 |
| 0204 09 V2-12h.009 | 0.204 | 31.8 |
| 0204 10 V2-24h.010 | 0.095 | 15.4 |
| 0204 11 V2-48h.011 | 0.115 | 40 |
| 0204 12 V3-Predose.012 | 0.293 | 50 |
| 0204 13 V3-Postdose.013 | 0.165 | 35.3 |
| 0204 14 V3-12h.014 | 0.049 | 40 |
| 0204 15 V3-24h.015 | 0.147 | 43.8 |
| 0204 16 V3-48h.016 | 0.043 | 42.9 |
| 0204 17 V4-Predose.017 | 0.046 | 28.6 |
| 0204 18 V4-Postdose.018 | 0.116 | 25 |
| 0204 19 V4-12h.019 | 0 | 0 |
| 0204 20 V4-24h.020 | 0.165 | 6.9 |
| 0204 21 V4-48h.021 | 0 | 0 |

Patient 0205 NNS

| | | |
|---|---|---|
| 0205 01 V1-Predose.001 | 0.101 | 7.69 |
| 0205 02 V1-Postdose.002 | 0.506 | 13.9 |
| 0205 03 V1-12h.003 | 0.221 | 0 |
| 0205 04 V1-24h.004 | 0.282 | 10.7 |
| 0205 05 V1-48h.005 | 0.014 | N/A |
| 0205 06 V1-72h.006 | 0.187 | 2.7 |
| 0205 07 V2-Predose.007 | 0.248 | 17.6 |
| 0205 08 V2-Posdose.008 | 0.463 | 3.23 |
| 0205 09 V2-12h.009 | 0.828 | 0 |
| 0205 10 V2-24h.010 | 0.452 | 3.92 |
| 0205 11 V2-48h.011 | 0.174 | 0 |
| 0205 12 V3-Predose.012 | 0.358 | 0 |
| 0205 13 V3-Postdose.013 | 2 | 0.897 |
| 0205 14 V3-12h.014 | 1.07 | 1.19 |
| 0205 15 V3-24h.015 | 1.33 | 1.3 |
| 0205 16 V3-48h.016 | 0.465 | 2.5 |
| 0205 17 V4-Predose.017 | 0.517 | 6.06 |
| 0205 18 V4-Postdose.018 | 1.02 | 1.75 |
| 0205 19 V4-12h.019 | 0.098 | 0 |
| 0205 20 V4-24h.020 | 0.132 | 12.5 |
| 0205 21 V4-48h.021 | 0.06 | 0 |

TABLE 13
Relative frequencies (%) of regulatory T cells and proliferating
Ki67+-Tregs in the blood of RA patients from third cohort
(10 mkg/kg)

| | |
|---|---|
| % FoxP3+/ | % Ki67+/ |
| CD4+ | FoxP3 + CD4+ |

Patient 0105 TIZ

| | | |
|---|---|---|
| RA0105 01 V1-Predose.001 | 0.297 | 29.2 |
| RA0105 02 V1-Postdose.002 | 0.342 | 22.6 |
| RA0105 03 V1-12h.003 | 0.079 | 16.7 |
| RA0105 04 V1-24h.004 | N/A | N/A |
| RA0105 05 V1-48h.005 | 0.514 | 19.6 |
| RA0105 06 V1-72h.006 | 0.875 | 22.1 |
| RA0105 07 V2-Predose.007 | 0.349 | 25.6 |
| RA0105 08 V2-Postdose.008 | 1.5 | 50 |
| RA0105 09 V2-12h.009 | N/A | N/A |
| RA0105 10 V2-24h.010 | 0.396 | 22.7 |
| RA0105 11 V2-48h.011 | 0.3 | 33.3 |
| RA0105 12 V3-Predose.012 | 0.947 | 15.3 |
| RA0105 13 V3-Postdose.013 | N/A | N/A |
| RA0105 14 V3-12h.014 | N/A | N/A |
| RA0105 15 V3-24h.015 | 0.096 | 0 |
| RA0105 16 V3-48h.016 | 0.12 | 20 |
| RA0105 17 V4-Predose.017 | N/A | N/A |
| RA0105 18 V4-Postdose.018 | N/A | N/A |
| RA0105 19 V4-12h.019 | N/A | N/A |
| RA0105 20 V4-24h.020 | 0.137 | 28.6 |
| RA0105 21 V4-48h.021 | 0.222 | 37.5 |

Patient 0206 LGK

| | | |
|---|---|---|
| 01 V1-Predose.001 | 0.653 | 36.6 |
| 02 V1-Postdose.002 | 0.045 | 12.5 |
| 03 V1-12h.003 | N/A | N/A |
| 04 V1-24h.004 | 2.08 | 25.8 |
| 05 V1-48h.005 | 1.48 | 26.8 |
| 06 V1-72h.006 | 1.46 | 44.6 |
| 07 V2-Predose.007 | 0.627 | 22.9 |
| 08 V2-Postdose.008 | 0.939 | 25.3 |
| 09 V2-12h.009 | N/A | N/A |
| 10 V2-24h.010 | 0.925 | 25.8 |
| 11 V2-48h.011 | 0.588 | 22.4 |
| 12 V3-Predose.012 | 0.71 | 26.4 |
| 13 V3-Postdose.013 | 0.792 | 21.5 |
| 14 V3-12h.014 | 0.815 | 57.1 |
| 15 V3-24h.015 | 0.401 | 54.1 |
| 16 V3-48h.016 | 0.128 | 35.7 |
| 17 V4-Predose.017 | 0.102 | 18.2 |
| 18 V4-Postdose.018 | 0.255 | 15.4 |
| 19 V4-12h.019 | 0.265 | N/A |
| 20 V4-24h.020 | 1.27 | 7.14 |
| 21 V4-48h.021 | 1.34 | 4.29 |

Patient 0104 TIZ

| | | |
|---|---|---|
| RA0104 01 V1-Predose.001 | 0.577 | 28.6 |
| RA0104 02 V1-Postdose.002 | 0.438 | 26.5 |
| RA0104 03 V1-12h.003 | 0.285 | 27.3 |
| RA0104 04 V1-24h.004 | 0.595 | 33.3 |
| RA0104 05 V1-48h.005 | 0.698 | 45.5 |
| RA0104 06 V1-72h.006 | 0.414 | 29.4 |
| RA0104 07 V2-Predose.007 | 0.212 | 75 |
| RA0104 08 V2-Postdose.008 | N/A | N/A |
| RA0104 09 V2-12h.009 | 1.14 | 57.1 |
| RA0104 10 V2-24h.010 | 0.798 | 58.1 |
| RA0104 11 V2-48h.011 | 1.96 | 61.1 |
| RA0104 12 V3-Predose.012 | 0.692 | 55.2 |
| RA0104 13 V3-Postdose.013 | 0.272 | 50 |
| RA0104 14 V3-12h.014 | 1.17 | 51.9 |
| RA0104 15 V3-24h.015 | 1.33 | 68.6 |
| RA0104 16 V3-48h.016 | 1.14 | 58.1 |
| RA0104 17 V4-Predose.017 | 0.48 | 70 |
| RA0104 18 V4-Postdose.018 | 0.271 | 37.5 |
| RA0104 19 V4-12h.019 | 1.36 | 52.9 |
| RA0104 20 V4-24h.020 | 0.878 | 56.4 |
| RA0104 21 V4-48h.021 | 1.09 | 70.9 |

N/A—not available

Conclusion and Discussion

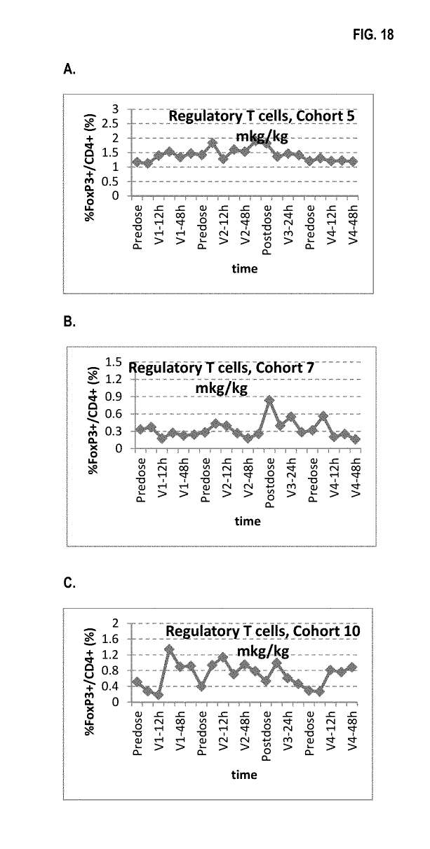

In general, heterogeneous data have been obtained probably due to patients' history, on-going therapies, age and severity of disease (FIG. 18, panels A-C).

T-regs % increase at postdose and 12-24 hours after infusion was observed in some patients, and the effect was more pronounced in the third highest-dose cohort. Slight increase of Tregs number was observed also in the third dose cohort. However there was no statistically significant evidence of dose-dependent effect.

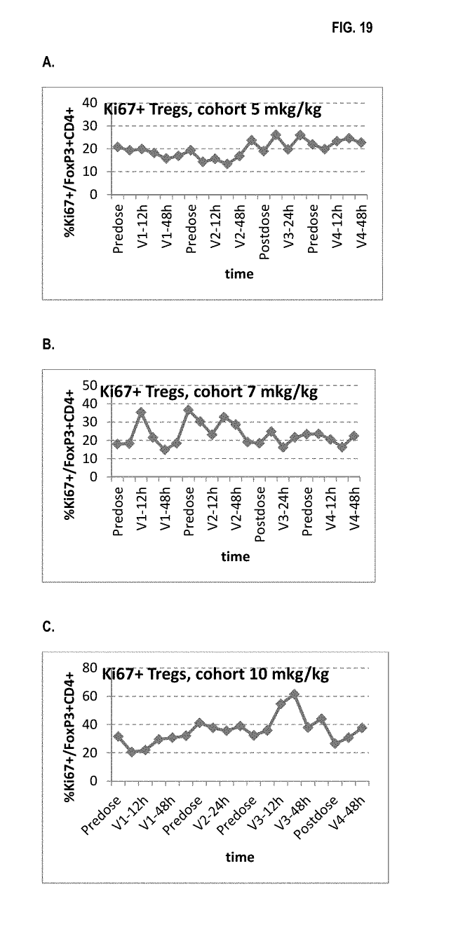

Also analysis of proliferation marker Ki67 showed increased number of Ki67+ Tregs at 24 hours after second and third infusions in several patients, however no clear tendency was found (FIG. 19, panels A-C).

It is important to mention that regulatory T cell numbers in peripheral blood is relatively low—and consists of about 1% from the overall T-reg count in the body. Moreover, among all CD4+ cells in the blood T-regs are least represented, when comparing to tissue matrix. Increase of circulating T-regs quantity may be only a secondary effect of T-regulatory response in tissues, because, without wishing to be bound by theory, circulating blood T-regs are unable to respond to TAB08 exposure. For the moment it's not clear enough which TAB08 tissue concentration would induce T-regs proliferation and its appearance in the systemic circulation, which in turn will allow registering their elevation in systemic circulation. T-regs actively move from blood to tissues and react to different stimulus, which impedes faithful interpretation of available data. Nonetheless, available data could directly or approximately indicate T-reg activation after TAB08 exposure of RA patients.

Slight tendency of the increase of Tregs by the end of the course of TAB08 therapy was seen in RA patients suggesting that Tregs translocate to inflamed sites of joints. It is likely that Treg cells are activated and expanded in lymphoid organs, and only few of them transit for a short time via blood stream in search of inflamed sites. Accordingly, no great expansion the blood was expected (in contrast to healthy volunteers and the profound expansion of Tregs observed in vitro where activated cells cannot escape and stay in plates). Compared to majority of available clinical research data which point out a reduction of circulating Tregs during the course of RA disease, no decrease of these cells was registered in the presence of TAB08, suggesting a potential of TAB08 to activate and expand Tregs in patients. Potential increase of activated synovial Tregs results in a compensatory mechanism to counteract local inflammation thus improving the state of inflamed joints. Indeed in 8 out of 9 patients improvements of joints was observed. Positive action of TAB08 is also confirmed by increase of anti-inflammatory IL-10 production. Importantly, no increase of 11-17 (signature pro-inflammatory Th17 Cells cytokine) was observed.

### Study of the Effects of TAB08 on Cytokine Production

Cytokines Level in Blood Serum Assessment

During first clinical trial in London 2006 it was shown that bolus TAB08 administration in high dose (100 μg/kg dose was used, which is 10-fold higher than maximal dose in our clinical study) provoked massive release of pro-inflammatory cytokines in 6 out of 6 volunteers, with cytokines concentrations being more than a thousand-fold higher than normal levels.

In new clinical trials in Yaroslavl infusion of TAB08 in concentration 1.5-7 mkg/kg was performed in healthy volunteers. During that study cytokines analysis in blood plasma of healthy volunteers did not show any clinically significant cytokines concentrations elevation after TAB08 infusion.

In this trial IL-1β, IL-2, IL-4, IL-6, IL-10, IL-17, TNFα, IFNγ concentrations in RA patients' blood plasma were measured for pharmacodynamic assessment of TAB08 effect on cytokines concentrations. Besides the main pro-inflammatory cytokines (IL-1β, IL-2, IL-4, IL-6, IL-17, TNFα, IFNγ), which were elevated during the clinical trial in London 2006, levels of the anti-inflammatory cytokine IL-10 was also measured in this trial.

Cytokines concentrations measurement was performed with flow cytometry method with high-sensitivity multiplex fluorescent analysis using antibody-coated spheres, specific to each cytokine. This method allows to measure cytokine concentrations in blood plasma with a sensitivity range of 0.274-200 pg/mL or higher. For this analysis blood samples were taken directly before TAB08 infusion (0 time point), and then after 1, 4, 8, 12 and 48 hours after each 4 infusions. Samples (3 mL) were collected in tubes and then centrifuged (3000×g×10 min). Plasma was stored at −70° C. until analysis. Biosamples transportation from study site to the laboratory was done in isothermic shipping containers with dry ice.

Blood plasma samples from 9 volunteers were available for cytokine measurement analysis. For all of them there was enough data for statistical analysis.

In 7 enrolled patients initial levels of IL-1β, IL-2, IL-4, IL-6, IL-10, IL-17, TNFα, and IFNγ during the trial were within physiological normal ranges, and in two patients IL-6 was increased. RA often associated with increased levels of IL-6 in the blood.

TABLE 14
Normal cytokine levels measured by high-sensitivity
multiplex fluorescent analysis in human plasma measured
by high-sensitivity multiplex fluorescent analysis

| Cytokine | Normal level (pg/mL) |
|---|---|
| IFNγ | <10.9 |
| TNFα | <3.7 |
| IL-10 | <3.7 |
| IL-6 | <5.6 |
| IL-4 | <4.1 |
| IL-2 | <4.9 |
| IL-1β | <48 |
| IL-17 | <9.4 |

Statistical data processing for comparison of cytokine concentrations was performed at different time points after TAB08 administration, a time point before TAB08 administration (predose time point) was included.

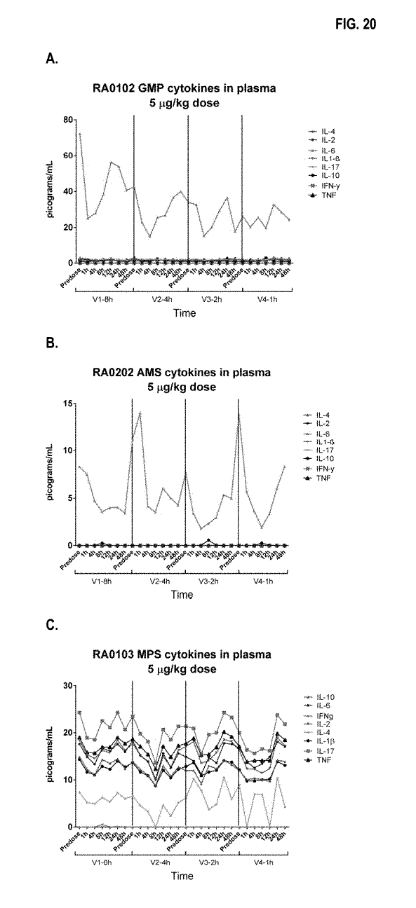

Data for each individual volunteer are presented. For example, data for the first dose cohort is shown in FIGS. 20, panels A-C. As shown, slightly elevated initial level of IL-6 was observed in one patient. Except this patient, levels of all cytokines at any time point after TAB08 administration are within physiological ranges and are not clinically significant. There was a decrease of IL-6 at 4 h compared to predose levels in 2 patients (FIG. 20, panels A-C).

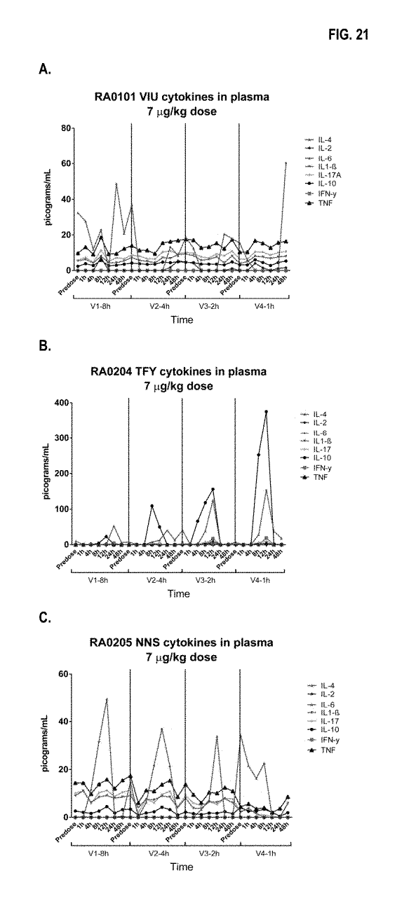

Data for the second dose cohort is shown in FIG. 21, panels A-C. As shown, slightly elevated initial level of IL-6 was observed in one patient. Significant increase of IL-6 at 4, 8 and 12 hours compared to predose level was registered in two patients. Significant increase of IL-10 at 8 and 12 hours compared to predose level was registered in two patients, and effect was more pronounced after 4^{th} infusion compared to first infusion. Weak activation of IL-10 secretion could also be detected in other patient (FIG. 21, panels A-C). Except IL-6 and IL-10, levels of all other cytokines at any time point after TAB08 administration are within physiological ranges and are not clinically significant.

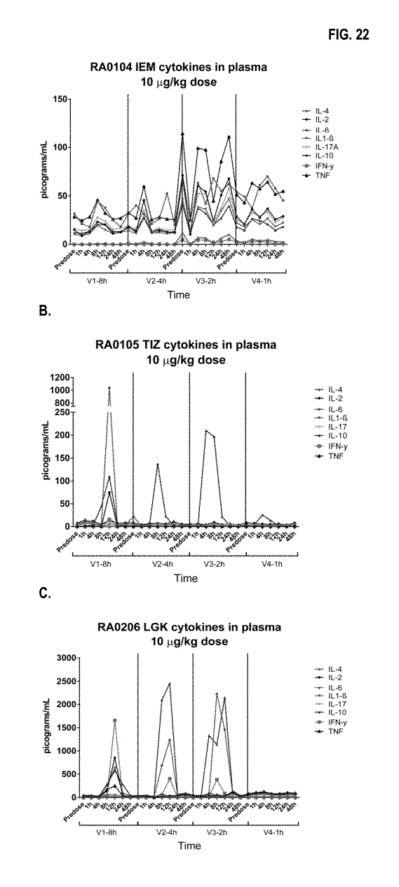

Data for the second dose cohort is shown in FIG. 22 panels A-C. As shown, significant increase of IL-6 at 8 and 12 hours compared to predose level was registered in all three patients. Significant increase of IL-10 at 4, 8 and 12 hours compared to predose level was registered in two patients, and effect was more pronounced at the third infusion compared to first infusion. Significant activation of IL-2 12 h after first infusion was observed in two patients, and the effect was still observed but weaker in one patient. Significant activation of IL-2, TNFα, IFN gamma production 12 hours after first infusion could be detected in one patient, effect was less pronounced after second and third infusions (FIG. 2, panels A-C). Importantly premedication with methylprednisolon on the fourth week effectively prevented release of all cytokines measured with an exception of IL-10 in one patient. Of note IL-10 is associated with an anti-inflammatory effect.

Conclusion and Discussion

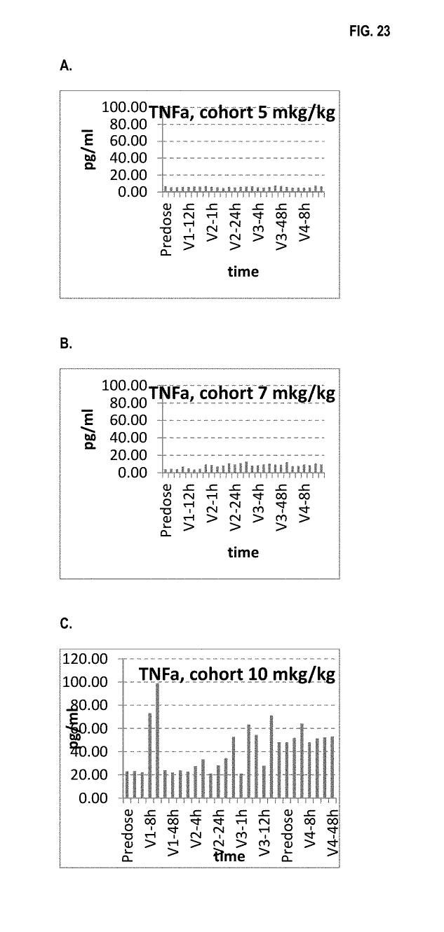

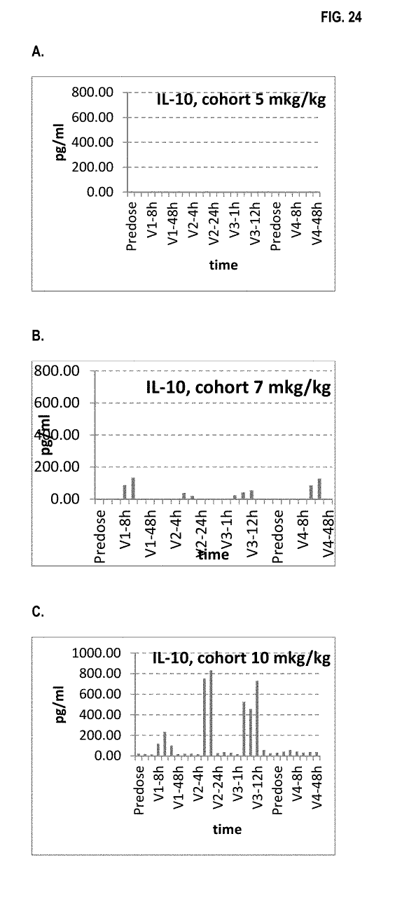

In general, during this study cytokine analysis in blood plasma of RA patients demonstrated dose-dependent increase of key inflammatory and anti-inflammatory cytokines (FIGS. 23-25). Clinically significant elevation of IL-10 was observed (FIG. 24, panels A-C). The effect is dose-dependent and more pronounced in third dose cohort (10 mkg/kg). Similar effect is observed for IL-6. There was no clinically important elevation of other pro-inflammatory cytokine (IL-1β, IL-2, IL-4, IL-17, TNFα, and IFNγ) levels in all patients except one when comparing to baseline concentrations before TAB08 administration for any TAB08 dose assessed in this trial.

It is important to mention significant elevation of the anti-inflammatory cytokine IL-10 concentrations within time range 4-24 hours after TAB08 infusion, with a maximum at time point around “12 hours”, when comparing to 0 time point (predose) (FIG. 24, panels A-C). Additionally weak activation of IL-2 could be observed in third high dose cohort. This could be considered as a biomarker of pharmacodynamic activity of TAB08. When comparing IL-10 concentrations differences between cohorts, a statistically significant elevation of the IL-10 concentration was seen at 12 hours after TAB08 administration at doses 10 μg/kg compared to 5 μg/kg. This finding could be treated as a trend for a dose-dependent elevation of the concentration of the anti-inflammatory cytokine IL-10 by TAB08 infusion.

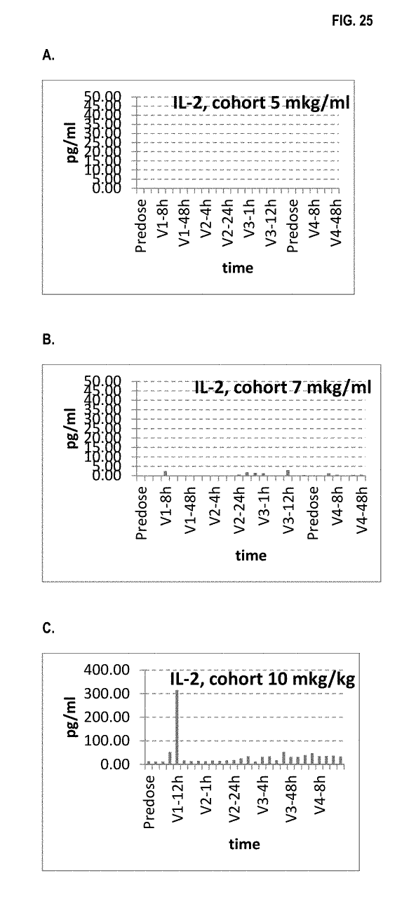

IL-10 is associated with an anti-inflammatory effect and short-term IL-10 production may correlate with active at time of TAB08 infusion T-regulatory lymphocytes. Among cell types which are involved in IL-10 production only T-cells have CD28 receptor. Among T-cells, T-regulatory lymphocytes are the main source of IL-10, but it can also be generated by Th2 and TR1 CD4 T-cells. Importantly, increased IL-2 production can be also seen in the third cohort (FIG. 25, panels A-C), and IL-2 is necessary for the activation of regulatory T cells. The absence of clear production of IL-2 in first cohort may partially be due to the ability of Treg cells to act as an IL-2 “sink”, a notion that is further supported by the increased levels of IL-10 in the blood of TAB-08 treated patients.

In Vitro Analysis in Restore System

The purpose of the RESTORE method application was definition of safety parameters of the use of TAB08 antibody and the possibility of participation of RA patients in clinical trials, in particular:

To determine the ability of peripheral blood mononuclear cells of RA patients to release cytokine release in the presence and absence of TAB08 antibody, which allows assessing the hypersensitivity of the cells to TAB08 monoclonal antibody and ensuring that significant inflammatory processes do not occur in the RA patients.

To determine the levels of response of peripheral blood mononuclear cells of RA patients to various concentrations of TAB08, i.e. trace the dose-dependence of cytokine release by mononuclear cells exposed to TAB08 in vitro.

To determine the ability of methylprednisolone, which has an anti-inflammatory effect, to suppress TAB08-induced cytokine release.

Screening Based On Restore System

Overall screening procedures were made in 2-3 stages.

Screening visit 1—verification of subject compliance with inclusion criteria to the study and absence of exclusion criteria 1-12.

Screening visit 2 was made after investigators obtained results of all tests made on screening visit 1 to take blood samples for RESTORE tests and verification of exclusion criteria Ng 13 and14.

Screening visit 3—made if it was necessary to repeat standard laboratory tests. Standard laboratory blood tests were valid for only 14 days.

If a subject met all inclusion criteria including RESTORE tests and did not have any exclusion criteria, he was enrolled to the study for study therapy.

Blood plasma samples from 9 volunteers were available for cytokine measurement analysis. For 8 of them there was enough data for statistical analysis.

The results of analyzes for the individual volunteers are presented below. It should be noted that the maximum doses of TAB08, used for the infusion of the volunteers in the third cohort was 10 μg/kg, which approximately corresponds to a concentration of TAB08 of 0.1 mkg/ml in in vitro experiments in the RESTORE system.

Cytokine production by PBMC samples taken from RA patients and
treated with increasing concentrations of TAB08 in the presence
or absence of MP

| | | | | | |
|---|---|---|---|---|---|
| Results File | IFNg | IL-2 | TNFa | IL-10 | IL-6 |

First Cohort (5 mkg/kg)
pg/ml

| | | | | | |
|---|---|---|---|---|---|
| RA 03.2 MPS Unstim.011 | 0.00 | 0.00 | 0.00 | 0.00 | 154.98 |
| RA 03.2 MPS TAB 08 1 μg_ml.012 | 0.00 | 0.00 | 60.67 | 0.00 | 395.95 |
| RA 03.2 MPS TAB 08 0.3 μg_ml.013 | 1.20 | 0.00 | 52.69 | 0.00 | 322.40 |
| RA 03.2 MPS TAB 08 0.1 μg_ml.014 | 0.00 | 0.00 | 9.37 | 0.00 | 59.39 |
| RA 03.2 MPS TAB 08 .03 μg_ml.015 | 0.00 | 0.00 | 0.00 | 0.00 | 2.68 |
| RA 03.2 MPS TAB 08 .01 μg_ml.016 | 0.00 | 0.00 | 0.00 | 0.00 | 0.00 |
| RA 03.2 MPS OKT 3 1 μg_ml.017 | 137.90 | 0.00 | 736.72 | 41.11 | 1094.34 |
| RA 03.2 MPS TAB 08 1 + MP.018 | 0.00 | 0.00 | 0.00 | 0.00 | 0.00 |
| RA 03.2 MPS TAB 08 0.3 + MP.019 | 0.00 | 0.00 | 0.00 | 0.00 | 0.00 |
| RA 03.2 MPS TAB 08 0.1 + MP.020 | 0.00 | 0.00 | 0.00 | 0.00 | 0.00 |
| RA 03.2 MPS TAB 08 0.03 + MP.021 | 0.00 | 0.00 | 0.00 | 0.00 | 0.00 |
| RA 03.2 MPS TAB 08 0.01 + MP.022 | 0.00 | 0.00 | 0.00 | 0.00 | 0.00 |
| RA 02.1 GMP Unstim.023 | 0.00 | 0.00 | 2.69 | 0.00 | 0.00 |
| RA 02.1 GMP TAB 08 1 μg_ml.024 | 0.00 | 0.00 | 0.00 | 0.00 | 0.00 |
| RA 02.1 GMP TAB 08 0.3 μg_ml.025 | 0.00 | 0.00 | 0.00 | 0.00 | 0.00 |
| RA 02.1 GMP TAB 08 0.1 μg_ml.026 | 0.00 | 0.00 | 0.00 | 0.00 | 0.00 |
| RA 02.1 GMP TAB 08 .03 μg_ml.027 | 0.00 | 0.00 | 0.00 | 0.00 | 0.00 |
| RA 02.1 GMP TAB 08 .01 μg_ml.028 | 0.00 | 0.00 | 0.00 | 0.00 | 0.00 |
| RA 02.1 GMP OKT 3 1 μg_ml.029 | 0.00 | 0.00 | 9.84 | 0.00 | 0.00 |
| RA 02.1 GMP TAB 08 1 + MP.030 | 0.00 | 0.00 | 0.00 | 0.00 | 0.00 |
| RA 02.1 GMP TAB 08 0.3 + MP.031 | 0.00 | 0.00 | 0.00 | 0.00 | 0.00 |
| RA 02.1 GMP TAB 08 0.1 + MP.032 | 0.00 | 0.00 | 0.00 | 0.00 | 0.00 |
| RA 02.1 GMP TAB 08 0.03 + MP.033 | 0.00 | 0.00 | 0.00 | 0.00 | 0.00 |
| RA 02.1 GMP TAB 08 0.01 + MP.034 | 0.00 | 0.00 | 0.00 | 0.00 | 0.00 |

Second Cohort (7 mkg/kg)
pg/ml

| RA 01.1 VIU Unstim.011 | 0.00 | 0.00 | 0.00 | 0.00 | 0.00 |
|---|---|---|---|---|---|
| RA 01.1 VIU TAB 08 1 μg_ml.012 | 0.00 | 0.00 | 0.00 | 0.00 | 0.00 |
| RA 01.1 VIU TAB 08 0.3 μg_ml.013 | 0.30 | 0.00 | 0.00 | 0.00 | 0.00 |
| RA 01.1 VIU TAB 08 0.1 μg_ml.014 | 0.00 | 0.00 | 0.00 | 0.00 | 0.00 |
| RA 01.1 VIU TAB 08 .03 μg_ml.015 | 0.00 | 0.00 | 0.00 | 0.00 | 0.00 |
| RA 01.1 VIU TAB 08 .01 μg_ml.016 | 0.54 | 0.00 | 1.22 | 0.00 | 0.00 |
| RA 01.1 VIU OKT3 1 μg_ml.017 | 83.39 | 0.00 | 378.86 | 0.00 | 0.00 |
| RA 01.1 VIU TAB 08 1 + MP.018 | 2.91 | 0.00 | 0.00 | 0.00 | 0.00 |
| RA 01.1 VIU TAB 08 0.3 + MP.019 | 0.00 | 0.00 | 0.00 | 0.00 | 0.00 |
| RA 01.1 VIU TAB 08 0.1 + MP.020 | 0.00 | 0.00 | 0.00 | 0.00 | 0.00 |
| RA 01.1 VIU TAB 08 0.03 + MP.021 | 0.00 | 0.00 | 0.00 | 0.00 | 0.00 |
| RA 01.1 VIU TAB 08 0.01 + MP.022 | 0.00 | 0.00 | 0.00 | 0.00 | 0.00 |
| TFY | | | | | |
| Unstimulated.011 | 0.00 | 0.00 | 0.00 | 0.00 | 0.00 |
| TAB08 1 μg_mL.012 | 0.00 | 0.00 | 0.00 | 19.98 | 0.00 |
| TAB08 0.3 μg_mL.013 | 0.00 | 0.00 | 0.00 | 10.80 | 0.00 |
| TAB08 0.1 μg_mL.014 | 0.00 | 0.00 | 0.00 | 0.00 | 0.00 |
| TAB08 0.03 μg_mL.015 | 0.00 | 0.00 | 0.00 | 0.00 | 0.00 |
| TAB08 0.01 μg_mL.016 | 0.00 | 0.00 | 0.00 | 19.36 | 0.00 |
| OKT3 1 μg_mL.017 | 242.40 | 992.52 | 27.75 | 566.99 | 0.00 |
| TAB08 1 + MP.018 | 0.00 | 0.00 | 0.00 | 10.80 | 0.00 |
| TAB08 0.3 + MP.019 | 0.01 | 0.00 | 0.00 | 28.54 | 0.00 |
| TAB08 0.1 + MP.020 | 0.00 | 0.00 | 0.00 | 1.78 | 0.00 |
| TAB08 0.03 + MP.021 | 0.00 | 0.00 | 0.00 | 19.56 | 0.00 |
| TAB08 0.01 + MP.022 | 0.00 | 0.00 | 0.00 | 0.00 | 0.00 |
| NNS Results File | IFNg | TNFa | IL-10 | IL-6 | IL-2 |
| TAB08 1 ug_mL.011 | 30.22 | 1015.22 | 4.85 | 43.58 | 0.00 |
| TAB08 0.3 ug_mL.012 | 47.27 | 656.17 | 10.94 | 58.96 | 0.00 |
| TAB08 0.1 ug_mL.013 | 19.04 | 177.04 | 0.00 | 6.90 | 0.00 |
| TAB08 0.03 ug_mL.014 | 22.40 | 39.97 | 0.00 | 0.00 | 0.00 |
| TAB08 0.01 ug_mL.015 | 11.33 | 16.61 | 0.00 | 0.00 | 0.00 |
| Unstimulated.016 | 0.00 | 1.61 | 0.00 | 0.00 | 0.00 |
| OKT3 1 ug_mL.017 | 45.95 | 458.03 | 0.00 | 40.62 | 0.00 |
| TAB08 1 + MP.018 | 19.40 | 17.36 | 0.00 | 0.00 | 0.00 |
| TAB08 0.3 + MP.019 | 36.38 | 39.30 | 1.58 | 0.00 | 0.00 |
| TAB08 0.1 + MP.020 | 23.19 | 33.68 | 0.19 | 0.00 | 0.00 |
| TAB08 0.03 + MP.021 | 16.33 | 15.52 | 0.00 | 0.00 | 0.00 |
| TAB08 0.01 + MP.022 | 22.40 | 31.95 | 0.26 | 0.00 | 0.00 |

Third Cohort (10 mkg/kg)
pg/ml

| | | | | | |
|---|---|---|---|---|---|
| RA04.1 IEM TAB08 1 ug_mL.011 | 0.00 | 0.00 | 0.00 | 0.00 | 204.47 |
| RA04.1 IEM TAB08 0.3 ug_mL.012 | 16.73 | 0.00 | 13.38 | 1.96 | 198.01 |
| RA04.1 IEM TAB08 0.1 ug_mL.013 | 12.86 | 0.00 | 18.24 | 3.16 | 199.07 |
| RA04.1 IEM TAB08 0.03 ug_mL.014 | 0.00 | 0.00 | 0.00 | 0.00 | 194.86 |
| RA04.1 IEM TAB08 0.01 ug_mL.015 | 0.00 | 0.00 | 0.00 | 0.00 | 191.77 |
| RA04.1 IEM Unstimulated.016 | 0.00 | 0.00 | 0.00 | 0.00 | 196.96 |
| RA04.1 IEM OKT3 1 ug_mL.017 | 128.54 | 0.00 | 387.62 | 1.76 | 503.89 |
| RA04.1 IEM TAB08 1 + MP.018 | 1.34 | 0.00 | 0.00 | 0.00 | 202.29 |
| RA04.1 IEM TAB08 0.3 + MP.019 | 0.00 | 0.00 | 0.00 | 0.00 | 198.01 |
| RA04.1 IEM TAB08 0.1 + MP.020 | 0.00 | 0.00 | 0.00 | 0.00 | 222.83 |
| RA04.1 IEM TAB08 0.03 + MP.021 | 0.00 | 0.00 | 0.00 | 0.00 | 221.63 |
| RA04.1 IEM TAB08 0.01 + MP.022 | 0.00 | 0.00 | 0.00 | 0.00 | 186.72 |
| RA05.1 IIZ TAB08 1.033 | 0.00 | 0.00 | 153.44 | 0.00 | 0.00 |
| RA05.1 IIZ TAB08 0.3.034 | 6.62 | 0.00 | 122.55 | 2.46 | 0.00 |
| RA05.1 IIZ TAB08 0.1.035 | 4.99 | 0.00 | 22.93 | 3.57 | 0.00 |
| RA05.1 IIZ TAB08 0.03.036 | 0.00 | 0.00 | 0.00 | 0.00 | 0.00 |
| RA05.1 IIZ TAB08 0.01.037 | 11.67 | 0.00 | 10.88 | 0.00 | 0.00 |
| RA05.1 IIZ unstimulated.038 | 0.00 | 0.00 | 0.00 | 0.00 | 0.00 |
| RA05.1 IIZ OKT3 1.039 | 234.56 | 0.00 | 1854.22 | 25.53 | 0.00 |
| RA05.1 IIZ TAB08 1 + MP.040 | 0.00 | 0.00 | 0.00 | 0.00 | 0.00 |
| RA05.1 IIZ TAB08 0.3 + MP.041 | 14.11 | 0.00 | 11.49 | 4.20 | 0.00 |
| RA05.1 IIZ TAB08 0.1 + MP.042 | 9.41 | 0.00 | 9.14 | 1.02 | 0.00 |
| RA05.1 IIZ TAB08 0.03 + MP.043 | 14.53 | 0.00 | 15.39 | 0.00 | 0.00 |
| RA05.1 IIZ TAB08 0.01 + MP.044 | 0.00 | 0.00 | 0.00 | 0.00 | 0.00 |
| RA 06.2 LGK TAB 08 1 μg_ml.011 | 0.00 | 0.00 | 62.79 | 0.00 | 0.00 |
| RA 06.2 LGK TAB 08 0.3 μg_ml.012 | 0.00 | 0.00 | 45.44 | 0.00 | 0.00 |
| RA 06.2 LGK TAB 08 0.1 μg_ml.013 | 0.00 | 0.00 | 0.00 | 0.00 | 0.00 |
| RA 06.2 LGK TAB 08 .03 μg_ml.014 | 0.00 | 0.00 | 0.00 | 0.00 | 0.00 |
| RA 06.2 LGK TAB 08 .01 μg_ml.015 | 0.00 | 0.00 | 0.00 | 0.00 | 0.00 |
| RA 06.2 LGK TAB 08 Unstim.016 | 0.00 | 0.00 | 0.00 | 0.00 | 0.00 |
| RA 06.2 LGK TAB 08 OKT3.017 | 405.30 | 0.00 | 680.89 | 0.00 | 0.00 |
| RA 06.2 LGK TAB 08 1 + MP.018 | 0.00 | 0.00 | 0.00 | 0.00 | 0.00 |
| RA 06.2 LGK TAB 08 .3 + MP.019 | 0.00 | 0.00 | 0.00 | 0.00 | 0.00 |
| RA 06.2 LGK TAB 08 .1 + MP.020 | 0.00 | 0.00 | 0.00 | 0.00 | 0.00 |
| RA 06.2 LGK TAB 08 .03 + MP.021 | 0.00 | 0.00 | 0.00 | 0.00 | 0.00 |
| RA 06.2 LGK TAB 08 .01 + MP.022 | 0.00 | 0.00 | 0.00 | 0.00 | 0.00 |

Conclusion

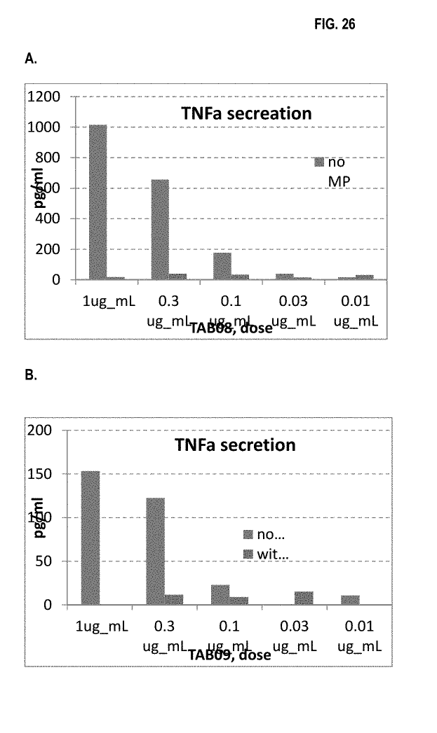

In seven examined volunteers the levels of IL-2, IL-6, IL-10, TNFα and IFN-γ in unstimulated cells remained within low physiological norms and in one patient initial high level of IL-6 was observed (Table 15). Four samples showed response, and two out of eight analyzed samples have responded significantly to high doses of TAB08. This is probably due to immune-suppressive therapy with methotrexate of the patients which may lead to inhibition of T cell activation. In all responders dose-dependent release of TNFα was observed (see FIG. 26, panels A-B).

The ability of methylprednisolone to inhibit cytokine release in the presence of high doses of TAB08 was also demonstrated for both responding patients (FIG. 26, panels A-B). Importantly similar situation was observed in clinics when premedication of patients from third cohort on 4^{th} infusion effectively suppressed cytokines release.

The maximum dose of TAB08 in this clinical study was 10 μg/kg which approximately corresponds to a TAB08 concentration of 100 ng/ml in in vitro experiments. It should be noted that during the cells stimulation in in vitro experiments using the TAB08 dose of 1000 ng/ml, which corresponds to the concentration of antibody in the first clinical study TGN1412 in London in 2006 (volunteers were administered a bolus dose of 0.1 mg/kg, i.e. 100 μg/kg, which is 10 times higher than the maximum dose used in this study), peripheral blood mononuclear cells of 4 RA patients demonstrated release of proinflammatory cytokines such as TNFα that may be an in vitro predictor of the “cytokine storm” syndrome.

The data obtained in the present clinical trial allows us to consider the RESTORE system as an adequate mean for determining individual sensitivity of mononuclear cells to stimulation with TAB08 drug. In view of the obtained data it is assumed that the RESTORE method allows to adequately perform the selection of patients for therapy with TAB08 in the planned clinical studies.

Measurement of Regulatory T Cells in RESTORE System

PBMC were cultured in supplemented RPMI 1640 containing 10% AB-positive heat-inactivated human serum (Sigma) for two days at high cell density (HD; 1×10⁷/ml) to allow tissue-like interactions. Cells were then harvested and cultured under standard conditions (1×10⁶/ml) in 96 or 48-well flat-bottom tissue culture plates in a final volume of 0.2 or 0.6 ml in a humidified incubator at 37° C. with 5% CO2. Intracellular Ki67 staining was used to measure cell proliferation. For intracellular staining of Foxp3, Ki67 and CD4, cells were first surface stained, permeabilized with Fix/Perm (eBioscience), and stained with the appropriate antibodies diluted in Perm/Wash (eBioscience). FACS analysis was performed using a FACSCalibur flow cytometer (BD Biosciences). Data were analyzed using FlowJo software (TreeStar).

TABLE 16
Relative frequencies (%) of regulatory T cells and proliferating
Ki67+-Tregs in in PBMC samples taken from RA patients and
treated with increasing concentrations of TAB08 in the presence
or absence of MP.

| | |
|---|---|
| % FoxP3+/CD4+ | % Ki67+/FoxP3+ CD4+ |

Patient 0104 IEM

| | | |
|---|---|---|
| Unstimulated | 0.052 | 50 |
| TAB08 1 μg_mL. | 2.15 | 63.9 |
| TAB08 0.3 μg_mL | 1.4 | 63.3 |
| TAB08 0.1 μg_mL | 1.07 | 46.9 |
| TAB08 0.03 μg_mL | 0.494 | 48.1 |
| TAB08 0.01 μg_mL | 0.09 | 25 |
| OKT3 1 μg_mL | 4.96 | 84.9 |
| TAB08 1 + MP | 0.246 | 52.4 |
| TAB08 0.3 + MP | 0.109 | 80 |
| TAB08 0.1 + MP | 0.097 | 75 |
| TAB08 0.03 + MP | 0.053 | N/A |
| TAB08 0.01 + MP | 0.056 | 60 |

Patient 0105 TIZ

| | | |
|---|---|---|
| TAB08 1 μg_mL | 7.0 | 94.7 |
| TAB08 0.3 μg_mL | 6.8 | 94.4 |
| TAB08 0.1 μg_mL | 4.7 | 84.9 |
| TAB08 0.03 μg_mL | 2.0 | 52.7 |
| TAB08 0.01 μg_mL | 0.4 | 44.3 |
| Unstimulated | 0.5 | 38.4 |
| OKT3 1 μg_mL | 5.1 | 80.5 |
| TAB08 1 + MP | 4.1 | 85.1 |
| TAB08 0.3 + MP | 3.3 | 72.1 |
| TAB08 0.1 + MP | 1.3 | 72.9 |
| TAB08 0.03 + MP | 0.6 | 88.0 |
| TAB08 0.01 + MP | 0.8 | 88.4 |

Patient 0204 TFY

| | | |
|---|---|---|
| Unstimulated | 0.522 | 4.27 |
| TAB08 1 μg_mL | 1.17 | 37 |
| TAB08 0.3 μg_mL | 1.37 | 37.2 |
| TAB08 0.1 μg_mL | 1.11 | 21.6 |
| TAB08 0.03 μg_mL | 0.653 | 11.5 |
| TAB08 0.01 μg_mL | 0.25 | 12.5 |
| OKT3 1 μg_mL | 4.52 | 77.7 |
| TAB08 1 + MP | 0.443 | 9.18 |
| TAB08 0.3 + MP | 0.512 | 7.23 |
| TAB08 0.1 + MP | 0.611 | 15.2 |
| TAB08 0.03 + MP | 0.881 | 9 |
| TAB08 0.01 + MP | 0.198 | 6.82 |

Patient 0205 NNS

| | | |
|---|---|---|
| Unstimulated | 0.412 | 52.2 |
| TAB08 1 μg_mL | 3.76 | 77.8 |
| TAB08 0.3 μg_mL | 3.57 | 70.9 |
| TAB08 0.1 μg_mL | 3.47 | 61.8 |
| TAB08 0.03 μg_mL | 1.62 | 32 |
| TAB08 0.01 μg_mL | 0.613 | 27.9 |
| OKT3 1 μg_mL | 4.62 | 67.7 |
| TAB08 1 + MP | 3.06 | 77.3 |
| TAB08 0.3 + MP | 3.28 | 77.2 |
| TAB08 0.1 + MP | 1.4 | 50.3 |
| TAB08 0.03 + MP | 1.03 | 53.1 |
| TAB08 0.01 + MP | 0.953 | 57 |

Patient 0206

| | | |
|---|---|---|
| TAB08 1 ug_mL | 5.98 | 92.1 |
| TAB08 0.3 ug_mL | 5.32 | 92.1 |
| TAB08 0.1 ug_mL | 3.87 | 84.3 |
| TAB08 0.03 ug_mL | 2.51 | 47.9 |
| TAB08 0.01 ug_mL | 0.786 | 15.6 |
| unstimulated | 0.299 | 16.2 |
| OKT3 1 ug_ml | 2.79 | 80.8 |
| TAB08 1 + MP | 2.6 | 87.6 |
| TAB08 0.3 + MP | 1.49 | 77.9 |
| TAB08 0.1 + MP | 0.923 | 60.7 |
| TAB08 0.03 + MP | 0.021 | 50 |
| TAB08 0.01 + MP | 0.022 | 0 |

Conclusion

In all the samples analyzed significant increase of regulatory T cells was observed in dose-dependent manner (Table 16). Proliferation of regulatory T cells was observed as registered by Ki67 staining. It is important to mention that while only two out of eight analyzed patients have responded significantly to TAB08 in terms of cytokine release, while all the samples demonstrated increase of regulatory T cells by approx. 10 times.

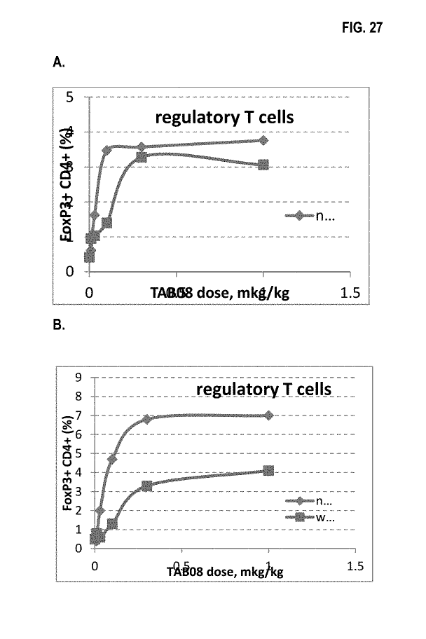

The ability of methylprednisolone (MP) to inhibit cytokine release in the presence of high doses of TAB08 was also demonstrated in this study. Notably while MP effectively suppresses cytokines production, regulatory T cells maintain their to proliferate in most of the samples in the presence of MP (FIG. 27, panels A-B).

### Discussion and Conclusions

Pharmacodynamic Data

Discussion on the pharmacodynamic of TAB08 is based on the data obtained during the present study from the analysis of regulatory T-lymphocytes and concentration of pro-and anti-inflammatory cytokines in the blood of the participants of the clinical study.

Induction of Tregs by TAB08, as measured in the blood, presented a considerable inter individual variability. No statistically significant dose-dependent response of regulatory T-lymphocytes to TAB08 was revealed, analyzing the degree of increase of % T-regulatory lymphocytes compared with the baseline levels. However, it is important to mention that regulatory T cell numbers in peripheral blood is relatively low—and consists of about 1% from the overall T-reg count in the body. Moreover, among all CD4+ cells in the blood T-regs are least represented, when comparing to tissue matrix. Increase of circulating T-regs quantity may be only a secondary effect of T-regulatory response in tissues, because, without wishing to be bound by theory, circulating blood T-regs are unable to respond to TAB08 exposure. For the moment it's not clear enough which TAB08 tissue concentration would induce T-regs proliferation and its appearance in the systemic circulation, which in turn will allow registering their elevation in systemic circulation. T-regs actively move from blood to tissues and react to different stimulus, which impedes faithful interpretation of available data. Nonetheless, available data could directly or approximately indicate T-reg activation after TAB08 exposure of RA patients.

Indeed the majority of patients included in the analysis presented a slight increase on the concentration of regulatory T-lymphocytes in the blood. The maximum increase in the concentration of regulatory T-lymphocytes in most of the volunteers was noted on 12-24 h post TAB08 infusion for some patients.

This observation is further supported by in vitro Restore test in which dramatic increase in Treg cells in response to TAB08 was observed. Inclusion of TAB08 led to a dose-dependent increase in the relative and absolute number of activated Treg cells, which was apparent with as little as 0.01 μg/mL of the antibody, suggesting their preferential expansion in response to the TAB08. PBMC cultures were stimulated for 5 days with graded concentrations of TAB08 and evaluated the presence of activated Treg cells. Treg cells were defined as a distinct subset of CD4 T cells expressing high levels of Foxp3 and CD25 and proliferating Tregs were analyzed using Ki67 marker. It was shown before that TAB08-induced increase in Treg cells is due to expansion of previously existing (presumably Treg) cells, and not due to conversion from conventional CD4 T-cells.

In all the analyzed cohorts a significant increase in the concentration of IL-6 and anti-inflammatory cytokine IL-10 was measured in the time range of 8-48 hours after the infusion of TAB08 with the peak at time point “12 hours”, in comparison with the point 0 (predose), which can be regarded as a sign of pharmacodynamic activity of TAB08.

There was no statistically significant elevation of other pro-inflammatory cytokine (IL-1β, IL-2, IL-4, IL-17, TNFα, and IFNγ) levels except one patient when comparing to baseline concentrations before TAB08 administration for any TAB08 dose assessed in this trial.

Additionally weak activation of IL-2 could be observed in third (10 mkg/kg) high dose cohort. Together with IL-10 this could be considered as a biomarker of pharmacodynamic activity of TAB08. When comparing IL-10 concentrations differences between cohorts, a statistically significant elevation of the IL-10 concentration was seen at 12 hours after TAB08 administration at doses 10 μg/kg compared to 5 μg/kg. This finding could be treated as a trend for a dose-dependent elevation of the concentration of the anti-inflammatory cytokine IL-10 by TAB08 infusion.

In general, the data obtained in the course of this study shows that the range of the TAB08 doses included is safe and efficient from the point of view of regulatory T cell subpopulation and release of pro- and anti-inflammatory cytokines.

Overall The results of laboratory tests and clinical examinations of 9 RA patients enrolled in the study to different dose cohorts and received TAB08 drug at a dose of 5 mkg/kg to 10 mkg/kg allow to make a conclusions on the good safety and efficacy of used TAB08 doses in RA patients, that justifies the conduction of further clinical trials of TAB08.

Pharmacokinetics

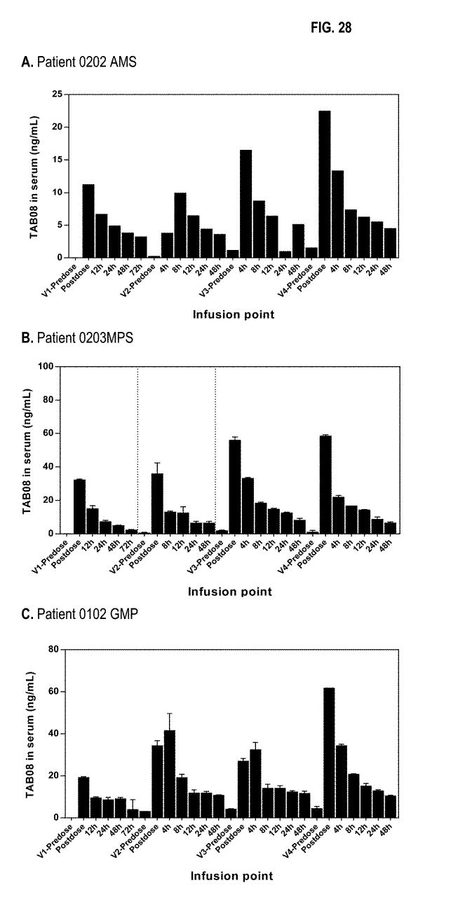

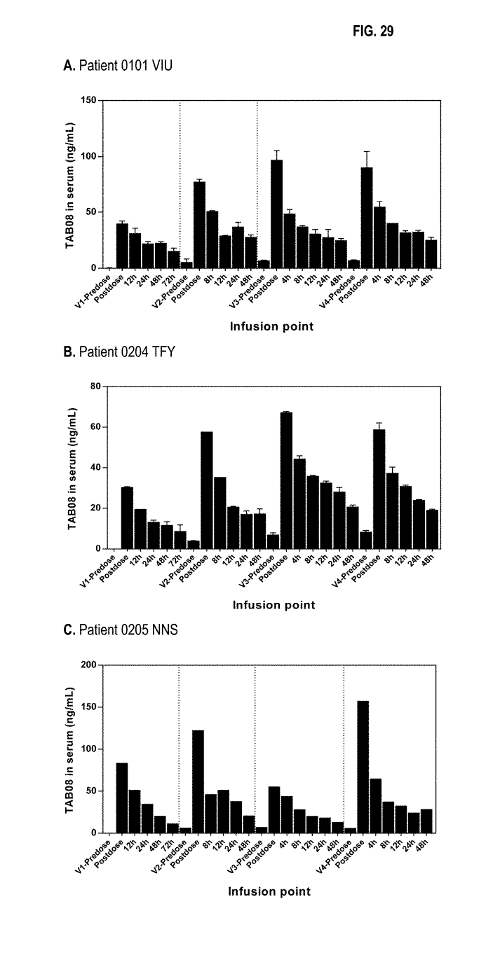

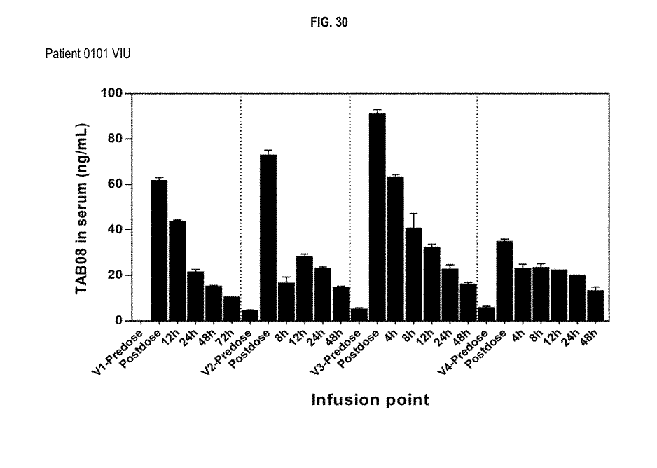

Evaluation of serum TAB08 PK was performed at various time points (FIGS. 28-30). Blood samples for PK evaluation were collected on pre-dose and postdose (immediately after infusion), 12, 24, 48 and 72 hours after the first infusion, and on pre-dose and postdose (immediately after infusion), 12, 24 and 48 hours after the second, third and fourth infusions. TAB08 level was measured by Cytometric Bead Array (BD) using Functional Beads (BD) conjugated to human CD28 protein. Patient serum sample is incubated with CD28-coated beads, the TAB08 present in serum binds CD28 on the beads with high affinity and this is detected with an anti-hlgG4-PE antibody and read with a FACSCalibur flow cytometer. PK parameters included: maximum observed plasma concentration (Cmax); area under the plasma concentration-time curve up to Week 4 and infinity (AUC0—Week 4 and AUC0→∞, respectively); terminal phase half-life; and clearance.

Conclusion

As seen in FIG. 28-30, peak plasma TAB08 concentrations were generally observed at the end of infusion (time point postdose). The serum concentrations were higher in the high-dose group than in the low-dose group. Cmax and AUC typically also increased proportional to dose, while concentrations declined in a bi-exponential fashion, with a terminal elimination half-life of approximately 7 days.

Accumulation due to repeated administration was observed in majority of the patients.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

1. A method for treating a patient having an inflammatory disease, comprising:
administering an anti-CD28 binding agent to said patient, in an amount effective to activate Treg cells, without inducing substantial release of pro-inflammatory cytokines.

2. (canceled)

3. The method of claim 1, wherein the inflammatory disease is characterized by an autoimmune condition.

4. (canceled)

5. The method of claim 3, wherein the inflammatory disease is rheumatoid arthritis.

6. The method of claim 5, wherein the rheumatoid arthritis is Stage I, Stage II, Stage III, or Stage IV.

7. The method of claim 5, wherein the rheumatoid arthritis is non-responsive to a corticosteroid, NSAID, COX-2 inhibitor, or biologic alone.

8. The method of claim 5, wherein the rheumatoid arthritis is in remission.

9. The method of claim 5, wherein the rheumatoid arthritis is active.

10. The method of of claim 1, wherein the anti-CD28 binding agent is a monoclonal antibody, or antigen-binding portion thereof.

11. The method of claim 10, wherein the anti-CD28 binding agent is a superagonist.

12. The method of claim 11, wherein the anti-CD28 binding agent induces preferential expansion of Treg cells.

13. The method of claim 10, wherein the anti-CD28 binding agent binds to an epitope of CD28 that competes with TAB08.

14. The method of claim 13, wherein the anti-CD28 binding agent is a monoclonal antibody or antigen-binding portion thereof, and wherein one or more CDR sequences are as in TAB08.

15. The method of claim 14, wherein the anti-CD28 binding agent is TAB08, or comprises a light chain or heavy chain sequence of TAB08.

16.-20. (canceled)

21. The method of claim 1, wherein the patient is administered with a composition comprising the anti-CD28 binding agent at a concentration of from about 0.5 mg/ml to about 10 mg/ml.

22. (canceled)

23. The method of claim 1, wherein the anti-CD28 binding agent is administered by intravenous infusion.

24. The method of claim 23, wherein the anti-CD28 binding agent is infused for a period of at least about 2 hours, or at least about 4 hours, or at least about 8 hours, or at least about 10 hours.

25. The method of claim 24, wherein the anti-CD28 binding agent is infused for no more than about 14 hours, about 12 hours, about 10 hours, about 8 hours, about 6 hours, or about 4 hours.

26.-31. (canceled)

31. The method of claim 1, wherein the anti-CD28 binding agent is administered at about 0.5 μg/kg to about 7 μg/kg of patient body weight, or about 1 μg/kg to about 6 μg/kg of patient body weight, or about 2 μg/kg to about 4 μg/kg of patient body weight.

32. The method of claim 1, wherein the anti-CD28 binding agent is administered about once per week, about once per month, about every other month, or about one to ten times per year, or about 4 to about 12 times per year.

33.-36. (canceled)

37. The method of claim 1, wherein the method provides at least about 20% improvement in the American College of Rheumatology (ACR) score.

38.-68. (canceled)
